(19) United States
(12) Patent Application Publication
(10) Pub. No.: US 2023/0013669 A1
(43) Pub. Date: Jan. 19, 2023

(54) Antibacterial Treatment Using a Cannabinoid and an Active Agent

(71) Applicant: Botanix Pharmaceuticals Limited, North Perth (AU)

(72) Inventors: Matthew Callahan, Philadelphia, PA (US); Michael Thurn, Kangaroobie (AU)

(21) Appl. No.: 17/778,910

(22) PCT Filed: Nov. 27, 2020
(86) PCT No.: PCT/AU2020/051283

§ 371 Date: May 23, 2022

(30) Foreign Application Priority Data
Nov. 29, 2019 (AU) 2019904529
Sep. 24, 2020 (AU) 2020903438

(51) Int. Cl. A61K 31/546; A61K 31/05; A61K 31/427; A61K 31/431

(52) U.S. Cl. CPC A61K 31/546; A61K 31/05; A61K 31/427; A61K 31/431

(57) ABSTRACT

A composition comprising a cannabinoid and a disruptor compound that removes or substantially removes or reduces the integrity of the outer membrane of a bacterium.

### TECHNICAL FIELD

The present invention relates to a composition for the treatment or prevention of bacterial infections, comprising a cannabinoid and a compound that removes or substantially removes or reduces the integrity of the outer membrane of a bacterium, and a method for use thereof.

### BACKGROUND ART

Compounds with antimicrobial properties have attracted great interest in recent times as a result of an increase in the prevalence of infections caused by bacteria, resulting in serious or fatal diseases. Furthermore, the regular use of broad spectrum antibiotic formulas has led to the increased occurrence of bacterial strains resistant to some antimicrobial compositions.

Novel antimicrobial compounds have the potential to be highly effective against these types of treatment-resistant bacteria. The pathogens, having not previously been exposed to the antimicrobial composition, may have little to no resistance to the treatment.

There is no indication that bacterial resistance to antibiotics will stop and for this reason new antibiotics and new treatment options are necessary to achieve a desirable treatment outcome in patients.

Gram-negative bacteria are particularly difficult to treat as the antibiotic must penetrate both the outer membrane and the bacterial membrane.

There is a need to provide new methods for the treatment of infections by bacteria, particularly bacteria resistant to the current antibiotic compounds available and particularly Gram-negative bacteria resistant to the current antibiotic compounds available. This invention seeks to provide such alternative treatment methods.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

The present invention provides a composition comprising a cannabinoid and a compound that removes or substantially removes or reduces the integrity of the outer membrane of a bacterium. Preferably the composition may be used for the treatment or prevention of an infection by a Gram-negative or Gram-positive bacterium. The present invention therefore provides a composition comprising a cannabinoid and a compound that removes or substantially removes or reduces the integrity of the outer membrane of a bacterium for the treatment or prevention of an infection by a Gram-negative or Gram-positive bacterium. The invention further provides a composition comprising cannabidiol and/or acids thereof and a compound that removes or substantially removes or reduces the integrity of the outer membrane of a bacterium for the treatment or prevention of an infection by a Gram-negative or Gram-positive bacterium.

The present invention further provides a method for the treatment or prevention of an infection by a bacterium in a subject in need of such treatment comprising the step of:
administering an effective amount of a composition comprising a cannabinoid and a compound that removes or substantially removes or reduces the integrity of the outer membrane of a bacteria.

The invention further provides a method for the treatment or prevention of an infection by a bacterium in a subject in need of such treatment comprising the step of:
administering an effective amount of a composition comprising cannabidiol and/or acids thereof and a compound that removes or substantially removes or reduces the integrity of the outer membrane of a bacteria.

The composition may be administered topically, orally, by injection, or by nasal or pulmonary administration.

According to another aspect of the invention, there is provided the use of a composition comprising a cannabinoid and a compound that removes or substantially removes or reduces the integrity of the outer membrane of a bacteria for the treatment or prevention of a bacterial infection in a subject in need of such treatment or prevention. The invention further provides the use of a composition comprising cannabidiol and/or acids thereof and a compound that removes or substantially removes or reduces the integrity of the outer membrane of a bacteria for the treatment or prevention of a bacterial infection in a subject in need of such treatment or prevention.

According to another aspect of the invention, there is provided the use of a cannabinoid and a compound that removes or substantially removes or reduces the integrity of the outer membrane of a bacteria, in the manufacture of composition for the treatment of an infection by a bacterium in a subject. The invention further provides the use of cannabidiol and/or acids thereof and a compound that removes or substantially removes or reduces the integrity of the outer membrane of a bacteria, in the manufacture of composition for the treatment of an infection by a bacterium in a subject.

According to another aspect of the invention, there is provided a kit comprising a cannabinoid and a compound that removes or substantially removes or reduces the integrity of the outer membrane of a bacteria for the treatment or prevention of a bacterial infection in a subject in need of such treatment or prevention and instructions for use in accordance with any one of the methods described herein. The invention further provides a kit comprising cannabidiol and/or acids thereof and a compound that removes or substantially removes or reduces the integrity of the outer membrane of a bacteria for the treatment or prevention of a bacterial infection in a subject in need of such treatment or prevention and instructions for use in accordance with any one of the methods described herein.

Preferably, the cannabinoid is chosen from the list comprising: cannabidiol, cannabinol, cannabigerol, cannabichromene, and Δ⁹-tetrahydrocannabinol. Preferably, the cannabinoid is chosen from the list comprising: cannabidiol, cannabinolic acid. Most preferably, the cannabinoid is cannabidiol.

Preferably, the compound that removes or substantially removes or reduces the integrity of the outer membrane of the bacteria is selected from the group consisting of: β-lactams, fosfomycin, lysozyme, polymyxins such as polymyxin B, lipopeptides including cyclic lipopeptides such as octapeptins, chelating agents such as ethylenediaminetetraacetic acid (EDTA), glycopeptides, tromethamine, diazaborine, protamine, ketodeoxyoctulosonate analogs, polylysine polymers such as pentalysine, polyornithine polymers, nourseothricin, defensins, cecropins, magainins, melittin, bactenecins, seminalplasmin, apidaecin, abaecin, bactericidal/permeability-increasing protein (BPI), eosinophil major basic protein, eosinophil cationic protein (ECP), lactoferrin, azurocidin, cathepsin G, aminoglycosides, Tris, nitrilotriacetate, sodium hexametaphosphate (HMP), acetylsalicylate, ascorbate, fleroxacin and other fluoroquinolones, monoglycerides such as monolaurin and monocaprin, and/or immunological agents.

### DESCRIPTION OF INVENTION

### Detailed Description of the Invention

### Cannabinoid

The term cannabinoid includes compounds which interact with the cannabinoid receptor and various cannabinoid mimetics, such as certain tetrahydropyran analogs (e.g., Δ⁹-tetrahydrocannabinol, Δ⁸-tetrahydro-cannabinol, 6,6,9-trimethyl-3-pentyl-6H-dibenzo[b,d]pyran-1-ol, 3-(1,1-dimethylheptyl)-6,6a,7,8,10,10a-hexahydro-1-hydroxy-6,6-dimethyl-9H-dibenzo[b,d]pyran-9-one, (−)-(3S,4S)-7-hydroxy-Δ6-tetrahydrocannabinol-1,1-dimethylheptyl, (+)-(3S,4S)-7-hydroxy-Δ6-tetrahydrocannabinol-1,1-dimethylheptyl, 11-hydroxy-Δ⁹-tetrahydrocannabinol, and Δ8-tetrahydrocannabinol-11-oic acid)); certain piperidine analogs (e.g., (−)-(6S,6aR,9R,10aR)-5,6,6a,7,8,9,10,10a-octahydro-6-methyl-3-[(R)-1-methyl-4-phenylbutoxy]-1,9-phenanthridinediol-1-acetate)); certain aminoalkylindole analogs (e.g., (R)-(+)-[2,3-dihydro-5-methyl-3-(-4-morpholinylmethyl)-pyrrolo[1,2,3-de]-1,4-benzoxazin-6-yl]-1-naphthalenyl-methanone); and certain open pyran ring analogs (e.g., 2-[3-methyl-6-(1-methylethenyl)-2-cyclohexen-1-yl]-5-pentyl-1,3-benzenediol and 4-(1,1-dimethylheptyl)-2,3′-dihydroxy-6′alpha-(3-hydroxypropyl)-1′,2′,3′,4′,5′,6′-hexahydrobiphenyl).

Cannabinoids contemplated by the present invention include:
Cannabidiol (such as 2-[(1S,6S)-3-methyl-6-prop-1-en-2-ylcyclohex-2-en-1-yl]-5-pentylbenzene-1,3-diol) and acids thereof (such as cannabidiolic acid-2,4-dihydroxy-3-[(1R,6R)-3-methyl-6-prop-1-en-2-ylcyclohex-2-en-1-yl]-6-pentylbenzoic acid);
Cannabinol (6,6,9-trimethyl-3-pentylbenzo[c]chromen-1-ol) and acids thereof (such as cannabinolic acid-1-hydroxy-6,6,9-trimethyl-3-pentylbenzo[c]chromene-2-carboxylic acid);
Δ⁹-tetrahydrocannabinol (such as (−)-Δ9-tetrahydrocannabinol) and acids thereof (such as Δ⁹-tetrahydrocannabinolic acid A-(6aR,10aR)-1-hydroxy-6,6,9-trimethyl-3-pentyl-6a,7,8,10a-tetrahydrobenzo[c]chromene-2-carboxylic acid);
11-hydroxy-Δ9-tetrahydrocannabinol (such as (±)-11-hydroxy-Δ9-tetrahydrocannabinol);
Cannabigerol (2-[(2E)-3,7-dimethylocta-2,6-dienyl]-5-pentylbenzene-1,3-diol) and acids thereof (such as cannabigerolic acid-3-[(2E)-3,7-dimethylocta-2,6-dienyl]-2,4-dihydroxy-6-pentylbenzoic acid);
Tetrahydrocannabivarin—(such as (6aR,10aR)-6,6,9-trimethyl-3-propyl-6a,7,8,10a-tetrahydrobenzo[c]chromen-1-ol) and acids thereof (such as tetrahydrocannabivarinic acid-(6aR,10aR)-1-hydroxy-6,6,9-trimethyl-3-propyl-6a,7,8,10a-tetrahydrobenzo[c]chromene-2-carboxylic acid);
Cannabichromene (2-methyl-2-(4-methylpent-3-enyl)-7-pentylchromen-5-ol);
Nantradol hydrochloride ((−)-(6S,6aR,9R,10aR)-5,6,6a,7,8,9,10,10a-octahydro-6-methyl-3-[(R)-1-methyl-4-phenylbutoxy]-1,9-phenanthridinediol-1-acetate);
Nabilone (3-(1,1-dimethylheptyl)-6,6a,7,8,10,10a-hexahydro-1-hydroxy-6,6-dimethyl-9H-dibenzo[b,d]pyran-9-one);
6,6,9-trimethyl-3-pentyl-6H-dibenzo[b,d]pyran-1-ol (such as (−)-Δ8- tetrahydrocannabinol);
(3S,4S)-7-hydroxy-Δ6-tetrahydrocannabinol-1,1-dimethylheptyl (such as (−)-(3S,4S)-7-hydroxy-Δ6-tetrahydrocannabinol-1,1-dimethylheptyl and (+)-(3S,4S)-7-hydroxy-Δ6-tetrahydrocannabinol-1,1-dimethylheptyl);
Δ8-tetrahydrocannabinol-11-oic acid ((6aR,10aR)-1-hydroxy-6,6-dimethyl-3-pentyl-6a,7,10,10a-tetrahydrobenzo[c]chromene-9-carboxylic acid);
(R)-(+)-[2,3-dihydro-5-methyl-3-(-4-morpholinylmethyl)-pyrrolo[1,2,3-de]-1,4-benzoxazin-6-yl]-1-naphthalenyl-methanone;
4-(1,1-dimethylheptyl)-2,3′-dihydroxy-6′alpha-(3-hydroxypropyl)-1′,2′,3′,4′,5′,6′-hexahydrobiphenyl;
(6aR,10aR)-9-(hydroxymethyl)-6,6-dimethyl-3-pentyl-6a,7,8,10a-tetrahydrobenzo[c]chromen-1-ol.

### Abbreviations

### Cannbidiol—CBD

Cannabidiolic acid—CBDA

### Cannabinol—CBN

Cannabinolic acid—CBNA
Δ⁹-tetrahydrocannabinol—THC
Δ⁹-tetrahydrocannabinolic acid A—THCA-A

### Cannabigerol—CBG

Cannabigerolic acid—CBGA

### Cannabichromene—CBC

### Tetrahydrocannabivarin—THCV

### Tetrahydrocannabivarinic Acid—THCVA

Preferably, the cannabinoid is chosen from the list comprising: cannabidiol, cannabinol, cannabigerol, cannabichromene, and Δ⁹-tetrahydrocannabinol. Preferably, the cannabinoid is chosen from the list comprising: cannabidiol, cannabinolic acid. Most preferably, the cannabinoid is cannabidiol.

Cannabidiol, as used herein, refers to 2-[3-methyl-6-(1-methylethenyl)-2-cyclohexen-1-yl]-5-pentyl-1,3-benzenediol. The synthesis of cannabidiol is described, for example, in Petilka et al., Helv. Chim. Acta, 52: 1102 (1969) and in Mechoulam et al., J. Am. Chem. Soc., 87:3273 (1965), which are hereby incorporated by reference.

### Composition

According to one aspect of the invention, there is provided a composition comprising a cannabinoid and a compound that removes or substantially removes or reduces the integrity of the outer membrane of a bacterium. Preferably the composition may be used for the treatment or prevention of an infection by a Gram-negative bacterium or Gram-positive bacterium. The present invention therefore provides a composition comprising a cannabinoid and a compound that removes or substantially removes or reduces the integrity of the outer membrane of a bacterium for the treatment or prevention of an infection by a Gram-negative or Gram-positive bacterium. The invention further provides a composition comprising cannabidiol and/or acids thereof and a compound that removes or substantially removes or reduces the integrity of the outer membrane of a bacterium for the treatment or prevention of an infection by a Gram-negative or Gram-positive bacterium.

Preferably, the cannabinoid is chosen from the list comprising: cannabidiol, cannabinol, cannabigerol, cannabichromene, and Δ⁹-tetrahydrocannabinol. Preferably, the cannabinoid is chosen from the list comprising: cannabidiol, cannabinolic acid. Most preferably, the cannabinoid is cannabidiol.

The cannabinoid should be administered together with a compound that removes or substantially removes or reduces the integrity of the outer membrane of the bacteria. Preferably the compound that removes or substantially removes or reduces the integrity of the outer membrane of the bacteria allows the OM of the bacteria to be disrupted or disorganised such that the permeability of the OM is increased.

Gram-positive bacteria lack an outer membrane but are surrounded by layers of peptidoglycan many times thicker than is found in the Gram-negatives.

The term “compound that removes or substantially removes or reduces the integrity of the outer membrane of the bacteria” or “disruptor compound” or “disruptor” refers to a compound that removes or substantially removes or reduces the integrity of the outer membrane of a Gram-negative bacteria. Although Gram-positive bacteria lack this outer membrane, the presence of the compound that removes or substantially removes or reduces the integrity of the outer membrane of a bacteria may still enhance the effect of the co-applied cannabinoid.

As an example, the compound that removes or substantially removes or reduces the integrity of the outer membrane of the bacteria is selected from the group consisting of: β-lactams, fosfomycin, lysozyme, polymyxins such as polymyxin B, lipopeptides including cyclic lipopeptides such as octapeptins, chelating agents such as ethylenediaminetetraacetic acid (EDTA), glycopeptides, tromethamine, diazaborine, protamine, ketodeoxyoctulosonate analogs, polylysine polymers such as pentalysine, polyornithine polymers, nourseothricin, defensins, cecropins, magainins, melittin, bactenecins, seminalplasmin, apidaecin, abaecin, bactericidal/permeability-increasing protein (BPI), eosinophil major basic protein, eosinophil cationic protein (ECP), lactoferrin, azurocidin, cathepsin G, aminoglycosides, Tris, nitrilotriacetate, sodium hexametaphosphate (HMP), acetylsalicylate, ascorbate, fleroxacin and other fluoroquinolones, monoglycerides (such as monocaprin, monolaurin, monomyristin, monopalmitin, and monostearin). Preferred disruptor compounds include ticarcillin, cefotetan, aztreonam, colistin, MCC_6442, Octapetin C4, Spero SPR206, Spero Potentiator SPR741, FADDI-287 and MCC_8980. The disruptor compound may alternatively be an immunological agent (such as an antibody or vaccine) that reduces the integrity of the outer membrane.

In one preferred embodiment the cannabinoid is administered together with a compound that removes or substantially removes or weakens the integrity of the outer membrane of a Gram-negative bacterium. The compound preferably increases the permeability of the outer membrane of the Gram-negative bacteria and preferably increases the efficacy of the co-administered cannabinoid against the Gram-negative bacterium. Alternatively, the cannabinoid is administered together with a compound that removes or substantially removes or weakens the integrity of the outer membrane of bacterium to a Gram-positive bacterium. The compound preferably increases the efficacy of the co-administered cannabinoid against the Gram-positive bacterium.

Preferably, if the compound that removes or substantially removes or reduces the integrity of the outer membrane of the bacteria is an antimicrobial, it is administered in a sub-lethal amount when it is used to remove or substantially remove or reduce the integrity of the outer membrane of the bacteria. By “sub-lethal”, it is meant that the compound is administered in an amount that is lower than the MIC for that antimicrobial for the bacteria being treated. For example, if the compound has an MIC of 0.1 ug/mL for Streptococcus pyogenes when used as an antimicrobial, it is used at less than 0.1 ug/mL when it is used as a compound that removes or substantially removes or reduces the integrity of the outer membrane of a bacteria. Alternatively, if the compound has an MIC of 1.5 ug/mL for Campylobacter jejuni when used as an antimicrobial, it is used at less than 1.5 ug/mL when it is used as a compound that removes or substantially removes or reduces the integrity of the outer membrane of a bacteria.

The disruptor compound that removes or substantially removes or reduces the integrity of the outer membrane of the bacteria may generally act in one of two manners to disrupt the outer membrane: (i) the disruptor compound interferes with bacterial cell wall synthesis and inhibits cross-linking of the peptidoglycan so that the outer cell membrane fails to form or forms incompletely; or (ii) competitively displaces divalent cations (Ca²⁺ and Mg²⁺) from the phosphate groups of membrane lipids, which leads to disruption of the outer cell membrane. It is believed that ticarcillin, cefotetan and aztreonam are examples of the first type of disruptor compound, and colistin, MCC_6442, Octapetin C4, Spero SPR206, Spero Potentiator SPR741, FADDI-287 and MCC_8980 are examples of the second type of disruptor compound.

The disruptor compound that removes or substantially removes or reduces the integrity of the outer membrane of the bacteria may act differently in relation to different bacteria. For example, monocaprin is active against Gram-positive bacteria as a result of destruction of the cell membrane. In contrast, the activity of monocaprin against Gram-negative bacteria is due to damage to lipopolysaccharides in the cell walls.

Preferably the disruptor compound has little or no bactericidal activity. For example, the polymyxin B nonapeptide lacks significant bactericidal activity. The antimicrobial activity of cannabidiol in the present invention may not require the presence of a second antimicrobial (to give synergy between the antimicrobials), but may rather simply be due to the permeabilization effect of the membrane disruptor allowing the cannabidiol to act on the bacterial cell.

The present invention provides a composition for the treatment or prevention of an infection by a Gram-negative bacteria, said composition comprising cannabidiol and a disruptor compound that removes or substantially removes or reduces the integrity of the outer membrane of a bacteria selected from the list below, at a ratio to the cannabidiol within the ranges below, wherein the Gram-negative bacteria is Escherichia coli and the ratio of cannabidiol to disruptor compound is chosen from the following:

| Disruptor | Ratio (cannabidiol:disruptor) |
|---|---|
| Ticarcillin | More than 1:0.06-less than 1:8000 |
| Colistin | More than 1:0.00097-less than 1:4 |
| MCC_6442 | More than 1:0.016-less than 1:4080 |
| Cefotetan | More than 1:0.002-less than 1:255 |
| Aztreonam | More than 1:0.00097-less than 1:127 |
| Octapetin C4 | More than 1:0.03-1:64 |
| Spero SPR206 | More than 1:0.0004-less than 1:4080 |
| Spero Potentiator SPR741 | More than 1:0.002-1:64 |
| FADDI-287 | 1:0.125-less than 1:510 |
| MCC_8980 | More than 1:0.03-1:2040 |

More preferably the invention provides for the treatment or prevention of an infection by Escherichia coli wherein the ratio of cannabidiol to disruptor compound is chosen from the following:

| Disruptor | Ratio (cannabidiol:disruptor) |
|---|---|
| Ticarcillin | More than 1:1-less than 1:1000 |
| Colistin | More than 1:0.01-less than 1:1 |
| MCC_6442 | More than 1:1-less than 1:1000 |
| Cefotetan | More than 1:1-less than 1:190 |
| Aztreonam | More than 1:1-less than 1:90 |
| Octapetin C4 | More than 1:0.7-1:64 |
| Spero SPR206 | More than 1:0.001-less than 1:500 |
| Spero Potentiator SPR741 | More than 1:0.5-1:64 |
| FADDI-287 | 1:0.125-less than 1:100 |
| MCC_8980 | More than 1:0.07-1 2040 |

More preferably the invention provides for the treatment or prevention of an infection by Escherichia coli wherein the ratio of cannabidiol to disruptor compound is chosen from the following:

| Disruptor | Ratio (cannabidiol:disruptor) |
|---|---|
| Ticarcillin | 1:32 |
| Colistin | 1:0.008-1:0.25 |
| MCC_6442 | 1:25-1:50 |
| Cefotetan | 1:127 |
| Aztreonam | 1:63 |
| Octapetin C4 | 1:0.125-1:64 |
| Spero SPR206 | 1:0.016-1:127 |
| Spero Potentiator SPR741 | 1:1-1:64 |
| FADDI-287 | 1:0.125-1:4 |
| MCC_8980 | 1:0.125- 1:2040 |

The present invention provides a composition for the treatment or prevention of an infection by a Gram-negative bacteria, said composition comprising cannabidiol and a disruptor compound that removes or substantially removes or reduces the integrity of the outer membrane of a bacteria selected from the list below, at a ratio to the cannabidiol within the ranges below, wherein the Gram-negative bacteria is Pseudomonas aeruginosa and the ratio of cannabidiol to disruptor compound is chosen from the following:

| Disruptor | Ratio (cannabidiol:disruptor) |
|---|---|
| Ticarcillin | More than 1:0.25-less than 1:32640 |
| Ceftazidime | More than 1:0.03-less than 1:4080 |
| Aztreonam | More than 1:0.03-less than 1:4080 |
| MCC_8980 | More than 1:0.125-1:16 |

More preferably the invention provides for the treatment or prevention of an infection by Pseudomonas aeruginosa wherein the ratio of cannabidiol to disruptor compound is chosen from the following:

| Disruptor | Ratio (cannabidiol:disruptor) |
|---|---|
| Ticarcillin | More than 1:1-less than 1 25000 |
| Ceftazidime | More than 1:1-less than 1:3000 |
| Aztreonam | More than 1:1-less than 1 2000 |
| MCC_8980 | More than 1:0.25-1:16 |

More preferably the invention provides for the treatment or prevention of an infection by Pseudomonas aeruginosa wherein the ratio of cannabidiol to disruptor compound is chosen from the following:

| Disruptor | Ratio (cannabidiol:disruptor) |
|---|---|
| Ticarcillin | 1:16320 |
| Ceftazidime | 1:2040 |
| Aztreonam | 1:2-1:4 |
| MCC_8980 | 1:0.5-1:16 |

The present invention provides a composition for the treatment or prevention of an infection by a Gram-negative bacteria, said composition comprising cannabidiol and a disruptor compound that removes or substantially removes or reduces the integrity of the outer membrane of a bacteria selected from the list below, at a ratio to the cannabidiol within the ranges below, wherein the Gram-negative bacteria is Acinetobacter baumannii and the ratio of cannabidiol to disruptor compound is chosen from the following:

| Disruptor | Ratio (cannabidiol:disruptor) |
|---|---|
| Cefuroxime | More than 1:0.25-less than 1:2 |
| Colistin | 1:0.004-1:510 |
| MCC_6442 | More than 1:0.015-1:8 |
| Aztreonam | More than 1:0.06-1:16320 |
| Cefepime | More than 1:0.06-less than 1:0.5 |
| Octapeptin C4 | More than 1:0.03-1:2 |
| Spero SPR206 | More than 1:0.002-less than 1:2040 |
| Spero Potentiator SPR741 | More than 1:0.015-1:64 |
| FADDI-287 | 1:0.125-less than 1:510 |
| MCC_8980 | More than 1:0.125-1:16 |

More preferably the invention provides for the treatment or prevention of an infection by Acinetobacter baumannii wherein the ratio of cannabidiol to disruptor compound is chosen from the following:

| Disruptor | Ratio (cannabidiol:disruptor) |
|---|---|
| Cefuroxime | More than 1:0.37-less than 1:1 |
| Colistin | 1:0.004-1:510 |
| MCC_6442 | More than 1:0.03-1:8 |
| Aztreonam | More than 1:0.09-1:16320 |
| Cefepime | More than 1:0.09-less than 1:0.25 |
| Octapeptin C4 | More than 1:0.06-1:2 |
| Spero SPR206 | More than 1:0.01-less than 1:500 |
| Spero Potentiator SPR741 | More than 1:0.07-1:64 |
| FADDI-287 | 1:0.125-less than 1:100 |
| MCC_8980 | More than 1:0.19-1:16 |

More preferably the invention provides for the treatment or prevention of an infection by Acinetobacter baumannii wherein the ratio of cannabidiol to disruptor compound is chosen from the following:

| Disruptor | Ratio (cannabidiol:disruptor) |
|---|---|
| Cefuroxime | 1:0.5 |
| Colistin | 1:0.004-1:510 |
| MCC_6442 | 1:0.06-1:8 |
| Aztreonam | 1:0.125-1:16320 |
| Cefepime | 1:0.125 |
| Octapeptin C4 | 1:0.125-1:2 |
| Spero SPR206 | 1:1-1:255 |
| Spero Potentiator SPR741 | 1:0.125-1:64 |
| FADDI-287 | 1:0.125-1:16 |
| MCC_8980 | 1:0.25-1:16 |

The present invention provides a composition for the treatment or prevention of an infection by a Gram-negative bacteria, said composition comprising cannabidiol and a disruptor compound that removes or substantially removes or reduces the integrity of the outer membrane of a bacteria selected from the list below, at a ratio to the cannabidiol within the ranges below, wherein the Gram-negative bacteria is Klebsiella pneumoniae and the ratio of cannabidiol to disruptor compound is chosen from the following:

| Disruptor | Ratio (cannabidiol:disruptor) |
|---|---|
| Colistin | More than 1:0.008-less than 1:1020 |
| MCC_6442 | More than 1:0.03-1:16 |
| Spero SPR206 | More than 1:0.002-less than 1:4080 |
| Spero Potentiator SPR741 | More than 1:0.016-1:32 |
| FADDI-287 | 1:0.125-less than 1:510 |
| MCC_8980 | More than 1:0.25-1:2 |

More preferably the invention provides the above composition for the treatment or prevention of an infection by Klebsiella pneumoniae wherein the ratio of cannabidiol to disruptor compound is chosen from the following:

| Disruptor | Ratio (cannabidiol:disruptor) |
|---|---|
| Colistin | More than 1:1-less than 1:750 |
| MCC_6442 | More than 1:0.5-1:16 |
| Spero SPR206 | More than 1:0.09-less than 1:100 |
| Spero Potentiator SPR741 | More than 1:0.5-1:32 |
| FADDI-287 | 1:0.125-less than 1:250 |
| MCC_8980 | More than 1:0.5-1:2 |

More preferably the invention provides for the treatment or prevention of an infection by Klebsiella pneumoniae wherein the ratio of cannabidiol to disruptor compound is chosen from the following:

| Disruptor | Ratio (cannabidiol:disruptor) |
|---|---|
| Colistin | 1:510 |
| MCC_6442 | 1:2-1:16 |
| Spero SPR206 | 1:0.062-1:1 |
| Spero Potentiator SPR741 | 1:1-1:32 |
| FADDI-287 | 1:0.125-1:16 |
| MCC_8980 | 1:1-1:2 |

The present invention provides a composition for the treatment or prevention of an infection by a Gram-negative bacteria, said composition comprising cannabidiol and a compound selected from the list below, at a ratio to the cannabidiol within the ranges below, wherein the Gram-negative bacteria is Escherichia coli and the ratio of cannabidiol to compound is chosen from the following:

| Compound | Ratio (cannabidiol:compound) |
|---|---|
| Ticarcillin | More than 1:0.06-less than 1:8000 |
| Colistin | More than 1:0.00097-less than 1:4 |
| MCC_6442 | More than 1:0.016-less than 1:4080 |
| Cefotetan | More than 1:0.002-less than 1:255 |
| Aztreonam | More than 1:0.00097-less than 1:127 |
| Octapetin C4 | More than 1:0.03-1:64 |
| Spero SPR206 | More than 1:0.0004-less than 1:4080 |
| Spero Potentiator SPR741 | More than 1:0.002-1:64 |
| FADDI-287 | 1:0.125-less than 1:510 |
| MCC_8980 | More than 1:0.03-1:2040 |

More preferably the invention provides for the treatment or prevention of an infection by Escherichia coli wherein the ratio of cannabidiol to compound is chosen from the following:

| Compound | Ratio (cannabidiol:compound) |
|---|---|
| Ticarcillin | More than 1:1-less than 1:1000 |
| Colistin | More than 1:0.01-less than 1:1 |
| MCC_6442 | More than 1:1-less than 1:1000 |
| Cefotetan | More than 1:1-less than 1:190 |
| Aztreonam | More than 1:1-less than 1:90 |
| Octapetin C4 | More than 1:0.7-1:64 |
| Spero SPR206 | More than 1:0.001-less than 1:500 |
| Spero Potentiator SPR741 | More than 1:0.5-1:64 |
| FADDI-287 | 1:0.125-less than 1:100 |
| MCC_8980 | More than 1:0.07-1:2040 |

More preferably the invention provides for the treatment or prevention of an infection by Escherichia coli wherein the ratio of cannabidiol to compound is chosen from the following:

| Compound | Ratio (cannabidiol:compound) |
|---|---|
| Ticarcillin | 1:32 |
| Colistin | 1:0.008-1:0.25 |
| MCC_6442 | 1:25-1:50 |
| Cefotetan | 1:127 |
| Aztreonam | 1:63 |
| Octapetin C4 | 1:0.125-1:64 |
| Spero SPR206 | 1:0.016-1:127 |
| Spero Potentiator SPR741 | 1:1-1:64 |
| FADDI-287 | 1:0.125-1:4 |
| MCC_8980 | 1:0.125-1:2040 |

The present invention provides a composition for the treatment or prevention of an infection by a Gram-negative bacteria, said composition comprising cannabidiol and a compound selected from the list below, at a ratio to the cannabidiol within the ranges below, wherein the Gram-negative bacteria is Pseudomonas aeruginosa and the ratio of cannabidiol to compound is chosen from the following:

| Compound | Ratio (cannabidiol:compound) |
|---|---|
| Ticarcillin | More than 1:0.25-less than 1:32640 |
| Ceftazidime | More than 1:0.03-less than 1:4080 |
| Aztreonam | More than 1:0.03-less than 1:4080 |
| MCC_8980 | More than 1:0.125-1:16 |

More preferably the invention provides for the treatment or prevention of an infection by Pseudomonas aeruginosa wherein the ratio of cannabidiol to compound is chosen from the following:

| Compound | Ratio (cannabidiol:compound) |
|---|---|
| Ticarcillin | More than 1:1-less than 1:25000 |
| Ceftazidime | More than 1:1-less than 1:3000 |
| Aztreonam | More than 1:1-less than 1:2000 |
| MCC_8980 | More than 1:0.25-1:16 |

More preferably the invention provides for the treatment or prevention of an infection by Pseudomonas aeruginosa wherein the ratio of cannabidiol to compound is chosen from the following:

| Compound | Ratio (cannabidiol:compound) |
|---|---|
| Ticarcillin | 1:16320 |
| Ceftazidime | 1:2040 |
| Aztreonam | 1:2-1:4 |
| MCC_8980 | 1:0.5-1:16 |

The present invention provides a composition for the treatment or prevention of an infection by a Gram-negative bacteria, said composition comprising cannabidiol and a compound selected from the list below, at a ratio to the cannabidiol within the ranges below, wherein the Gram-negative bacteria is Acinetobacter baumannii and the ratio of cannabidiol to compound is chosen from the following:

| Compound | Ratio (cannabidiol:compound) |
|---|---|
| Cefuroxime | More than 1:0.25-less than 1:2 |
| Colistin | 1:0.004-1:510 |
| MCC_6442 | More than 1:0.015-1:8 |
| Aztreonam | More than 1:0.06-1:16320 |
| Cefepime | More than 1:0.06-less than 1:0.5 |
| Octapeptin C4 | More than 1:0.03-1:2 |
| Spero SPR206 | More than 1:0.002-less than 1:2040 |
| Spero Potentiator SPR741 | More than 1:0.015-1:64 |
| FADDI-287 | 1:0.125-less than 1:510 |
| MCC_8980 | More than 1:0.125-1:16 |

More preferably the invention provides for the treatment or prevention of an infection by Acinetobacter baumannii wherein the ratio of cannabidiol to compound is chosen from the following:

| Compound | Ratio (cannabidiol:compound) |
|---|---|
| Cefuroxime | More than 1:0.37-less than 1:1 |
| Colistin | 1:0.004-1:510 |
| MCC_6442 | More than 1:0.03-1:8 |
| Aztreonam | More than 1:0.09-1:16320 |
| Cefepime | More than 1:0.09-less than 1:0.25 |
| Octapeptin C4 | More than 1:0.06-1:2 |
| Spero SPR206 | More than 1:0.01-less than 1:500 |
| Spero Potentiator SPR741 | More than 1:0.07-1:64 |
| FADDI-287 | 1:0.125-less than 1:100 |
| MCC_8980 | More than 1:0.19-1:16 |

More preferably the invention provides for the treatment or prevention of an infection by Acinetobacter baumannii wherein the ratio of cannabidiol to compound is chosen from the following:

| Compound | Ratio (cannabidiol:compound) |
|---|---|
| Cefuroxime | 1:0.5 |
| Colistin | 1:0.004-1:510 |
| MCC_6442 | 1:0.06-1:8 |
| Aztreonam | 1:0.125-1:16320 |
| Cefepime | 1:0.125 |
| Octapeptin C4 | 1:0.125-1:2 |
| Spero SPR206 | 1:1-1:255 |
| Spero Potentiator SPR741 | 1:0.125-1:64 |
| FADDI-287 | 1:0.125-1:16 |
| MCC_8980 | 1:0.25-1:16 |

The present invention provides a composition for the treatment or prevention of an infection by a Gram-negative bacteria, said composition comprising cannabidiol and a compound selected from the list below, at a ratio to the cannabidiol within the ranges below, wherein the Gram-negative bacteria is Klebsiella pneumoniae and the ratio of cannabidiol to compound is chosen from the following:

| Compound | Ratio (cannabidiol:compound) |
|---|---|
| Colistin | More than 1:0.008-less than 1:1020 |
| MCC_6442 | More than 1:0.03-1:16 |
| Spero SPR206 | More than 1:0.002-less than 1:4080 |
| Spero Potentiator SPR741 | More than 1:0.016-1:32 |
| FADDI-287 | 1:0.125-less than 1:510 |
| MCC_8980 | More than 1:0.25-1:2 |

More preferably the invention provides the above composition for the treatment or prevention of an infection by Klebsiella pneumoniae wherein the ratio of cannabidiol to compound is chosen from the following:

| Compound | Ratio (cannabidiol:compound) |
|---|---|
| Colistin | More than 1:1-less than 1:750 |
| MCC_6442 | More than 1:0.5-1:16 |
| Spero SPR206 | More than 1:0.09-less than 1:100 |
| Spero Potentiator SPR741 | More than 1:0.5-1:32 |
| FADDI-287 | 1:0.125-less than 1:250 |
| MCC_8980 | More than 1:0.5-1:2 |

More preferably the invention provides for the treatment or prevention of an infection by Klebsiella pneumoniae wherein the ratio of cannabidiol to compound is chosen from the following:

| Compound | Ratio (cannabidiol:compound) |
|---|---|
| Colistin | 1:510 |
| MCC_6442 | 1:2-1:16 |
| Spero SPR206 | 1:0.062-1:1 |
| Spero Potentiator SPR741 | 1:1-1:32 |
| FADDI-287 | 1:0.125-1:16 |
| MCC_8980 | 1:1-1:2 |

Preferably the Gram-negative bacterial infection to be treated or prevented is caused by a bacteria from the list comprising: Klebsiella spp., Acinetobacter spp., Pseudomonas spp., Escherichia spp., Enterobacter spp., Shigella spp., Salmonella spp., Campylobacter spp., Haemophilus spp., Aeromonas spp., Francisella spp., Yersinia spp., Bordetella spp., Corynebacterium spp., Citrobacter spp., Chlamydia spp., Brucella spp., Helicobacter spp. and Vibrio spp. and combinations thereof. More preferably, the Gram-negative bacterial infection to be treated or prevented is caused by a bacteria from the list comprising: Klebsiella pneumoniae, Acinetobacter baumannii, Pseudomonas aeruginosa, Escherichia coli, Enterobacter spp. Salmonella enterica, Campylobacter jejuni, Haemophilus influenza, Chlamydia trachomatis, and Helicobacter pylori.

The Gram-positive bacterial infection to be treated or prevented may be caused by a bacteria from the list comprising: Streptococcus spp., Peptostreptococcus spp., Clostridium spp., Listeria spp., Bacillus spp., Staphylococcus spp., Propionibacterium spp., Kocuria spp., and Corynebacterium spp., and combinations thereof. The Gram-positive bacterial infection to be treated or prevented may be caused by a bacteria from the list comprising: Streptococcus pyogenes, Clostridium botulinum, Clostridium perfringens, Clostridium tetani, Listeria monocytogenes, Bacillus anthracis, Bacillus cereus, Staphylococcus aureus, Propionibacterium acnes, and Corynebacterium diphtheriae.

Preferably the Gram-positive bacterial infection to be treated or prevented is caused by a bacteria from the list comprising: Escherichia spp., Acinetobacter spp., Klebsiella spp. and Pseudomonas spp. The Gram-positive bacterial infection to be treated or prevented may be caused by a bacteria from the list comprising: Escherichia coli, Acinetobacter baumannii, Klebsiella pneumoniae and Pseudomonas aeruginosa.

The compositions may contain more than one cannabinoid. For example, the composition of the present invention may contain a combination of two, three or more cannabinoids. In preferred forms, at least one such cannabinoid is cannabidiol.

The compositions may contain more than one compound that removes or substantially removes or reduces the integrity of the outer membrane of a bacteria. For example, the composition of the present invention may contain a combination of two, three or more compounds.

The term “infection” as used herein means colonization by a micro-organism and/or multiplication of a micro-organism, in particular, a bacterium and more particularly a Gram-negative bacterium. The infection may be unapparent or result in local cellular injury. The infection may be localized, subclinical and temporary or alternatively may spread by extension to become an acute or chronic clinical infection. The infection may also be a latent infection, in which the microorganism is present in a subject, however the subject does not exhibit symptoms of disease associated with the organism.

Preferably the composition of the present invention delivers a therapeutically effective amount of the cannabinoid to the subject.

The phrase “therapeutically effective amount” as used herein refers to an amount of the cannabinoid sufficient to inhibit bacterial growth associated with bacterial carriage or a bacterial infection. That is, reference to the administration of the therapeutically effective amount of a cannabinoid according to the methods or compositions of the invention refers to a therapeutic effect in which substantial bacteriocidal or bacteriostatic activity causes a substantial inhibition of the bacterial carriage or bacterial infection. The term “therapeutically effective amount” as used herein, refers to a nontoxic but sufficient amount of the composition to provide the desired biological, therapeutic, and/or prophylactic result. The desired results include elimination of bacterial carriage or reduction and/or alleviation of the signs, symptoms, or causes of a disease, or any other desired alteration of a biological system. An effective amount in any individual case may be determined by one of ordinary skill in the art using routine experimentation. In relation to a pharmaceutical composition, effective amounts can be dosages that are recommended in the modulation of a diseased state or signs or symptoms thereof. Effective amounts differ depending on the pharmaceutical composition used and the route of administration employed. Effective amounts are routinely optimized taking into consideration various factors of a particular patient, such as age, weight, gender, etc and the area affected by disease or disease-causing microorganisms.

As used herein, “treating” or “treatment” refers to inhibiting the disease or condition, i.e., arresting or reducing its development or at least one clinical or subclinical symptom thereof, for example reducing or eliminating a bacterial infection. “Treating” or “treatment” further refers to relieving the disease or condition, i.e., causing regression of the disease or condition or at least one of its clinical or subclinical symptoms. The benefit to a subject to be treated is either statistically significant or at least perceptible to the subject and/or the physician. In the context of treating a bacterial infection, the term treatment includes reducing or eliminating colonization by bacteria and/or multiplication of bacteria, preferably Gram-negative bacteria or Gram-positive bacteria.

In one form of the invention, reducing or eliminating colonization by bacteria means reducing or eliminating colonization by bacteria as measured by % bacteria killed.

In one form of the invention, reducing or eliminating colonization by bacteria means reducing or eliminating colonization by bacteria as measured by a log₁₀ reduction in bacteria.

### Method of Treatment

According to another aspect of the invention, there is provided a method for the treatment or prevention of an infection by a bacterium in a subject in need of such treatment comprising the step of:
administering an effective amount of a composition comprising a cannabinoid and a compound that removes or substantially removes or reduces the integrity of the outer membrane of a bacteria.

Preferably, the cannabinoid is chosen from the list comprising: cannabidiol, cannabinol, cannabigerol, cannabichromene, and Δ⁹-tetrahydrocannabinol. Preferably, the cannabinoid is chosen from the list comprising: cannabidiol, cannabinolic acid. Most preferably, the cannabinoid is cannabidiol.

Preferably the Gram-negative bacterial infection to be treated or prevented is caused by a bacteria from the list comprising: Klebsiella spp., Acinetobacter spp., Pseudomonas spp., Escherichia spp., Enterobacter spp., Shigella spp., Salmonella spp., Campylobacter spp., Haemophilus spp., Aeromonas spp., Francisella spp., Yersinia spp., Bordetella spp., Corynebacterium spp., Citrobacter spp., Chlamydia spp., Brucella spp., Helicobacter spp. and Vibrio spp. and combinations thereof. More preferably, the Gram-negative bacterial infection to be treated or prevented is caused by a bacterium from the list comprising: Klebsiella pneumoniae, Acinetobacter baumannii, Pseudomonas aeruginosa, Escherichia coli, Enterobacter spp. Salmonella enterica, Campylobacter jejuni, Haemophilus influenza, Chlamydia trachomatis, and Helicobacter pylori.

Preferably the Gram-positive bacterial infection to be treated or prevented is caused by a bacterium from the list comprising: Streptococcus spp., Peptostreptococcus spp., Clostridium spp., Listeria spp., Bacillus spp., Staphylococcus spp., Propionibacterium spp., Kocuria spp., and Corynebacterium spp., and combinations thereof. More preferably, the Gram-positive bacterial infection to be treated or prevented is caused by a bacteria from the list comprising: Streptococcus pyogenes, Clostridium botulinum, Clostridium perfringens, Clostridium tetani, Listeria monocytogenes, Bacillus anthracis, Bacillus cereus, Staphylococcus aureus, Propionibacterium acnes, and Corynebacterium diphtheriae

### Topical Infections

In one aspect, the composition used in the method for the treatment or prevention of an infection by a Gram-negative bacteria or Gram-positive bacterium in a subject in need of such treatment is a topical pharmaceutical composition for the treatment of an infection of a dermal or mucosal surface.

In one form of the invention, the infection is related to one or more of the following conditions: acne, rash, blisters, burns, itch, cellulitis, folliculitis, nail infections, boils, hair infections, scalp infections, impetigo, hemorrhoids, canker sore, gingivitis, periodontitis, vaginitis, nose lesions, swelling, cut, surgical incision, sunburn, cracked skin, and combinations thereof.

In one form of the invention, the infection is an acute bacterial skin and skin structure infection (ABSSSI) where the infection is related to one or more of the following conditions: cellulitis/erysipelas, wound infection, and major cutaneous abscess that have a minimum lesion surface area of approximately 75 cm².

In one form of the invention, the infection is a complicated skin and skin structure infection (cSSSI) where the infection involves deep subcutaneous tissues or needs surgery in addition to antimicrobial therapy.

In one form of the invention, the infection is a non-complicated or community acquired skin or skin structure infection.

The method for topical treatment may comprise the administration of a cannabinoid and a compound that removes or substantially removes or reduces the integrity of the outer membrane of a bacteria directly to a dermal or mucosal surface of the subject. Preferably, the cannabinoid is applied topically to the skin or mucosal membranes (oral, vaginal, rectal) of the subject. The use may comprise administering a cannabinoid and a compound that removes or substantially removes or reduces the integrity of the outer membrane of a bacteria to the skin or mucosal membranes (oral, vaginal, rectal) of a subject.

### Ocular Infections

In one aspect, the composition used in the method for the treatment or prevention of an infection by a Gram-negative bacteria or Gram-positive bacterium in a subject in need of such treatment is an ocular pharmaceutical composition for the treatment of an ocular infection.

Ocular infections can be divided into (i) infections affecting the cornea and conjunctiva; (ii) infections in the soft tissue surrounding the eye (ocular adnexa and orbit) which can involve the eye indirectly and can spread from the orbit into the brain; and (iii) infections inside the eye (endophthalmitis), often following penetrating ocular trauma or after intraocular surgery. All of the above infections may be treated by the present method of delivering a cannabinoid and a compound that removes or substantially removes or reduces the integrity of the outer membrane of a bacterium.

The method of ocular treatment may comprise the administration of a cannabinoid and a compound that removes or substantially removes or reduces the integrity of the outer membrane of a bacterium directly to an ocular surface of the subject. Preferably, the cannabinoid and the compound that removes or substantially removes or reduces the integrity of the outer membrane of a bacterium is applied topically to the eye of the subject. However, the method may comprise administering the cannabinoid and the compound that removes or substantially removes or reduces the integrity of the outer membrane of a bacterium via intraocular injection, scleral injection, slow release implant or other delivery method. The use may comprise administering a cannabinoid and a compound that removes or substantially removes or reduces the integrity of the outer membrane of a bacterium to the eye of a subject.

### Infections Treated by Oral Administration

In one aspect, the composition used in the method for the treatment or prevention of an infection by a Gram-negative bacteria or Gram-positive bacterium in a subject in need of such treatment is an oral pharmaceutical composition comprising a cannabinoid and a compound that removes or substantially removes or reduces the integrity of the outer membrane of a bacteria for the treatment of an infection. Any infection in a subject by a bacterium may be treated using an orally administered treatment method.

The oral treatment method may comprise the administration of a cannabinoid and a compound that removes or substantially removes or reduces the integrity of the outer membrane of a bacterium to the gastrointestinal (GI) tract of the subject. Preferably, the cannabinoid and the compound that removes or substantially removes or reduces the integrity of the outer membrane of a bacterium enters the blood stream via absorption in the GI tract and is systemically available to the subject. However, the oral treatment method may comprise administering the cannabinoid and the compound that removes or substantially removes or reduces the integrity of the outer membrane of a bacterium to the GI tract for a localised effect. The use may comprise administering a cannabinoid and a compound that removes or substantially removes or reduces the integrity of the outer membrane of a bacterium orally to a subject.

### Infections Treated by Injection

In one aspect, the composition used in the method for the treatment or prevention of an infection by a Gram-negative bacteria or Gram-positive bacterium in a subject in need of such treatment is an injected pharmaceutical composition for the treatment of an infection. Any infection in a subject by a bacterium may be treated using a method of treatment of injected cannabinoids.

The injection treatment method may be by intravenous injection, intramuscular injection, or intraperitoneal injection. The administration may be intraventricularly, intracranially, intracapsularly, intraspinally, or intracisternally. Preferably, the injection treatment method is by intravenous injection.

The method of injected cannabinoids may comprise the administration of a cannabinoid and a compound that removes or substantially removes or reduces the integrity of the outer membrane of a bacteria to the subject. Preferably, the cannabinoid and the compound that removes or substantially removes or reduces the integrity of the outer membrane of a bacteria enters the blood stream via IV administration or a subcutaneous bolus and is systemically available to the subject. The use may comprise a cannabinoid and a compound that removes or substantially removes or reduces the integrity of the outer membrane of a bacteria by injection to a subject.

### Infections Treated by Nasal or Pulmonary Administration

In one aspect, the composition used in the method for the treatment or prevention of an infection by a Gram-negative bacteria or Gram-positive bacterium in a subject in need of such treatment is a nasal or pulmonary pharmaceutical composition for the treatment of an infection. Any infection in a subject by a bacteria may be treated using a nasal or pulmonary delivered treatment method.

Preferably, infections of the nasal cavity, sinuses, respiratory tract and lungs are treated using a nasal or pulmonary treatment method. For example, the treatment method of the present invention may be used to treat: pneumonia; sinus infection; infections associated with cystic fibrosis; infections associated with asthma; infections associated with acute respiratory distress syndrome (ARDS); infections associated with pneumoconiosis; infections associated with interstitial lung disease (ILD). The nasal or pulmonary treatment method may comprise the administration of a cannabinoid and a compound that removes or substantially removes or reduces the integrity of the outer membrane of a bacteria to the nasal or pulmonary system of the subject. The cannabinoid and the compound that removes or substantially removes or reduces the integrity of the outer membrane of a bacteria may enter the blood stream via absorption in the nasal or pulmonary system and be systemically available to the subject. However, the cannabinoid dosing method may alternatively comprise administering the cannabinoid and the compound that removes or substantially removes or reduces the integrity of the outer membrane of a bacteria to the nasal or pulmonary system for a localised effect. The use may comprise nasal or pulmonary administration of a cannabinoid and a compound that removes or substantially removes or reduces the integrity of the outer membrane of a bacteria to a subject.

### Additional Antimicrobials

Other active agents may also be incorporated into the composition of the present invention. For example, additional antimicrobial agents such as antibacterials, antifungals etc may be incorporated.

For example, the composition may further comprise benzoyl peroxide, erythromycin, clindamycin, doxycycline or meclocycline.

Additional antimicrobial agents that can be used include, but are not limited to silver compounds (e.g., silver chloride, silver nitrate, silver oxide), silver ions, silver particles, iodine, povidone/iodine, chlorhexidine, 2-p-sulfanilyanilinoethanol, 4,4′-sulfinyldianiline, 4-sulfanilamidosalicylic acid, acediasulfone, acetosulfone, amikacin, amoxicillin, amphotericin B, ampicillin, apalcillin, apicycline, apramycin, arbekacin, aspoxicillin, azidamfenicol, azithromycin, aztreonam, bacitracin, bambermycin(s), biapenem, brodimoprim, butirosin, capreomycin, carbenicillin, carbomycin, carumonam, cefadroxil, cefamandole, cefatrizine, cefbuperazone, cefclidin, cefdinir, cefditoren, cefepime, cefetamet, cefixime, cefinenoxime, cefminox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, chloramphenicol, chlortetracycline, ciprofloxacin, clarithromycin, clinafloxacin, clindamycin, clomocycline, colistin, cyclacillin, dapsone, demeclocycline, diathymosulfone, dibekacin, dihydrostreptomycin, dirithromycin, doxycycline, enoxacin, enviomycin, epicillin, erythromycin, flomoxef, fortimicin(s), gentamicin(s), glucosulfone solasulfone, gramicidin S, gramicidin(s), grepafloxacin, guamecycline, hetacillin, imipenem, isepamicin, josamycin, kanamycin(s), leucomycin(s), lincomycin, lomefloxacin, lucensomycin, lymecycline, meclocycline, meropenem, methacycline, micronomicin, midecamycin(s), minocycline, moxalactam, mupirocin, nadifloxacin, natamycin, neomycin, netilmicin, norfloxacin, oleandomycin, oxytetracycline, p-sulfanilylbenzylamine, panipenem, paromomycin, pazufloxacin, penicillin N, pipacycline, pipemidic acid, Polymyxin, primycin, quinacillin, ribostamycin, rifamide, rifampin, rifamycin SV, rifapentine, rifaximin, ristocetin, ritipenem, rokitamycin, rolitetracycline, rosaramycin, roxithromycin, salazosulfadimidine, sancycline, sisomicin, sparfloxacin, spectinomycin, spiramycin, streptomycin, succisulfone, sulfachrysoidine, sulfaloxic acid, sulfamidochrysoidine, sulfanilic acid, sulfoxone, teicoplanin, temafloxacin, temocillin, tetracycline, tetroxoprim, thiamphenicol, thiazolsulfone, thiostrepton, ticarcillin, tigemonam, tobramycin, tosufloxacin, trimethoprim, trospectomycin, trovafloxacin, tuberactinomycin, vancomycin, azaserine, candicidin(s), chlorphenesin, dermostatin(s), filipin, fungichromin, mepartricin, nystatin, oligomycin(s), ciproflaxacin, norfloxacin, ofloxacin, pefloxacin, enoxacin, rosoxacin, amifloxacin, fleroxacin, temafloaxcin, lomefloxacin, perimycin A or tubercidin, and the like.

### Subject

The subject may be any subject capable of infection by a bacterium, particularly a Gram-negative bacteria or Gram-positive bacteria. The subject may be mammalian or avian. Preferably, the subject is selected from the group comprising human, canine, avian, porcine, bovine, ovine, equine, and feline. More preferably, the subject is selected from the group comprising human, bovine, porcine, equine, feline and canine; most preferably human.

### Use

According to another aspect of the invention, there is provided the use of a composition comprising a cannabinoid and a compound that removes or substantially removes or reduces the integrity of the outer membrane of a bacteria for the treatment or prevention of a bacterial infection in a subject in need of such treatment or prevention.

According to another aspect of the invention, there is provided the use of a cannabinoid and a compound that removes or substantially removes or reduces the integrity of the outer membrane of a bacteria, in the manufacture of composition for the treatment of an infection by a bacterium in a subject.

Preferably, the cannabinoid is chosen from the list comprising: cannabidiol, cannabinol, cannabigerol, cannabichromene, and Δ⁹-tetrahydrocannabinol. Preferably, the cannabinoid is chosen from the list comprising: cannabidiol, cannabinolic acid. Most preferably, the cannabinoid is cannabidiol.

Preferably, the compound that removes or substantially removes or reduces the integrity of the outer membrane of the bacteria is selected from the group consisting of: β-lactams, fosfomycin, lysozyme, polymyxins such as polymyxin B, lipopeptides including cyclic lipopeptides such as octapeptins, chelating agents such as ethylenediaminetetraacetic acid (EDTA), glycopeptides, tromethamine, diazaborine, protamine, ketodeoxyoctulosonate analogs, polylysine polymers such as pentalysine, polyornithine polymers, nourseothricin, defensins, cecropins, magainins, melittin, bactenecins, seminalplasmin, apidaecin, abaecin, bactericidal/permeability-increasing protein (BPI), eosinophil major basic protein, eosinophil cationic protein (ECP), lactoferrin, azurocidin, cathepsin G, aminoglycosides, Tris, nitrilotriacetate, sodium hexametaphosphate (HMP), acetylsalicylate, ascorbate, fleroxacin and other fluoroquinolones, monoglycerides (such as monocaprin, monolaurin, monomyristin, monopalmitin, and monostearin), and/or immunological agents.

### Delivery

In one embodiment of the invention, the cannabinoid and a compound that removes or substantially removes or reduces the integrity of the outer membrane of a bacteria is administered to the subject using a dosing regimen selected from the group consisting of: three times daily; two times daily; daily; every second day, every third day, once weekly; once fortnightly and once monthly.

In accordance with certain embodiments, the composition is administered regularly until treatment is obtained. In one preferred embodiment, the composition is administered to the subject in need of such treatment using a dosing regimen selected from the group consisting of: every hour, every 2 hours, every 3 hours, once daily, twice daily, three times daily, four times daily, five times daily, once weekly, twice weekly, once fortnightly and once monthly. However, other application schedules may be utilized in accordance with the present invention. Preferably, the composition of the treatment regimen is administered to the subject between one and five times per day, more preferably once or twice per day.

The compositions used in the treatment methods of the invention may be administered by injection, or prepared for oral, inhaled (pulmonary), nasal, ocular, or any other form of administration. Preferably the compositions are administered, for example, intravenously, subcutaneously, intramuscularly, intraorbitally, ophthalmically, intraventricularly, intracranially, intracapsularly, intraspinally, intracisternally, intraperitoneally, buccal, rectally, vaginally, intranasally or by aerosol administration.

The mode of administration is preferably suitable for the form in which the composition has been prepared. The mode of administration for the most effective response may be determined empirically and the means of administration described below are given as examples, and do not limit the method of delivery of the composition of the present invention in any way. All the above compositions are commonly used in the pharmaceutical industry and are commonly known to suitably qualified practitioners.

The compositions of the invention may optionally include pharmaceutically acceptable nontoxic excipients and carriers. As used herein, a “pharmaceutical carrier” is a pharmaceutically acceptable solvent, suspending agent, excipient or vehicle for delivering the cannabinoid and the compound that removes or substantially removes or reduces the integrity of the outer membrane of a bacterium to the subject. The carrier may be liquid or solid and is selected with the planned manner of administration in mind.

The composition of the invention may be selected from the group consisting of: an immediate release composition, a delayed release composition, a controlled release composition and a rapid release composition.

The composition of the invention may further comprise an anti-inflammatory agent (such as a corticosteroid). If the composition is a topical composition, an anticomedolyic agent (such as tretinoin), and/or a retinoid or derivative thereof may also be added.

The compositions described herein may be formulated by including such dosage forms in an oil-in-water emulsion, or a water-in-oil emulsion. In such a composition, the immediate release dosage form is in the continuous phase, and the delayed release dosage form is in a discontinuous phase. The composition may also be produced in a manner for delivery of three dosage forms as hereinabove described. For example, there may be provided an oil-in-water-in-oil emulsion, with oil being a continuous phase that contains the immediate release component, water dispersed in the oil containing a first delayed release dosage form, and oil dispersed in the water containing a third delayed release dosage form.

The compositions described herein may be in the form of a liquid composition. The liquid composition may comprise a solution that includes a therapeutic agent (e.g. a cannabinoid and a compound that removes or substantially removes or reduces the integrity of the outer membrane of a bacteria) dissolved in a solvent. Generally, any solvent that has the desired effect may be used in which the therapeutic agent dissolves and which can be administered to a subject. Generally, any concentration of therapeutic agent that has the desired effect can be used. The composition in some variations is a solution which is unsaturated, a saturated or a supersaturated solution. The solvent may be a pure solvent or may be a mixture of liquid solvent components. In some variations the solution formed is an in-situ gelling composition. Solvents and types of solutions that may be used are well known to those versed in such drug delivery technologies.

The composition may or may not contain water. Preferably, the composition does not contain water, i.e. it is non-aqueous. In another preferred embodiment, the composition does not comprise a preservative.

The administration of the cannabinoid and the compound that removes or substantially removes or reduces the integrity of the outer membrane of a bacteria in accordance with the methods and compositions of the invention may be by any suitable means that results in an amount sufficient to treat a microbial infection or to reduce microbial growth at the location of infection.

The cannabinoid and the compound that removes or substantially removes or reduces the integrity of the outer membrane of a bacteria may be contained in any appropriate amount and in any suitable carrier substance, and is generally present in an amount of 1-95% by weight of the total weight of the composition.

The pharmaceutical composition may be formulated according to the conventional pharmaceutical or veterinary practice (see, for example, Remington: The Science and Practice of Pharmacy, 20th edition, 2000, ed; A. R. Gennaro, Lippincott Williams & Wilkins, Philadelphia, and Encyclopedia of Pharmaceutical Technology, eds; J. Swarbrick and J. C. Boylan, 1988-1999, Marcel Dekker, New York; Remington's Pharmaceutical Sciences, 18^{th} Edition, Mack Publishing Company, Easton, Pa., USA).

Generally, examples of suitable carriers, excipients and diluents include, without limitation, water, saline, ethanol, dextrose, glycerol, lactose, dextrose, sucrose, sorbitol, mannitol, starches, gum acacia, calcium phosphates, alginate, tragacanth, gelatine, calcium silicate, microcrystalline cellulose, polyvinylpyrrolidone, cellulose, water syrup, methyl cellulose, methyl and propylhydroxybenzoates, polysorbates, talc magnesium stearate, mineral oil or combinations thereof. The compositions can additionally include lubricating agents, pH buffering agents, wetting agents, emulsifying and suspending agents, preserving agents, sweetening agents or flavouring agents.

The composition may be in the form of a controlled-release composition and may include a degradable or non-degradable polymer, hydrogel, organogel, or other physical construct that modifies the release of the cannabinoid. It is understood that such compositions may include additional inactive ingredients that are added to provide desirable colour, stability, buffering capacity, dispersion, or other known desirable features. Such compositions may further include liposomes, such as emulsions, foams, micelles, insoluble monolayers, liquid crystals, phospholipid dispersions, lamellar layers and the like. Liposomes for use in the invention may be formed from standard vesicle-forming lipids, generally including neutral and negatively charged phospholipids and a sterol, such as cholesterol.

### Topical Compositions

Compositions of the invention may be administered topically. Therefore, contemplated for use herein are compositions adapted for the direct application to the skin. Preferably, the topical composition comprises a cannabinoid and a compound that removes or substantially removes or reduces the integrity of the outer membrane of a bacterium.

The composition may be in a form selected from the group comprising suspensions, emulsions, liquids, creams, oils, lotions, ointments, gels, hydrogels, pastes, plasters, roll-on liquids, skin patches, sprays, glass bead dressings, synthetic polymer dressings and solids. For instance, the compositions of the invention may be provided in the form of a water-based composition or ointment which is based on organic solvents such as oils. Alternatively, the compositions of the invention may be applied by way of a liquid spray comprising film forming components and at least a solvent in which the cannabinoid and the compound that removes or substantially removes or reduces the integrity of the outer membrane of a bacteria are dispersed or solubilised.

The composition of the invention may be provided in a form selected from the group comprising, but not limited to, a rinse, a shampoo, a lotion, a gel, a leave-on preparation, a wash-off preparation, and an ointment.

Various topical delivery systems may be appropriate for administering the compositions of the present invention depending up on the preferred treatment method. Topical compositions may be produced by dissolving or combining the cannabinoid and the compound that removes or substantially removes or reduces the integrity of the outer membrane of a bacteria in an aqueous or non-aqueous carrier. In general, any liquid, cream, or gel or similar substance that does not appreciably react with the cannabinoid and the compound that removes or substantially removes or reduces the integrity of the outer membrane of a bacteria or any other of the active ingredients that may be introduced into the composition and which is non-irritating is suitable. Appropriate non-sprayable viscous, semi-solid or solid forms can also be employed that include a carrier compatible with topical application and have dynamic viscosity preferably greater than water.

Suitable compositions are well known to those skilled in the art and include, but are not limited to, solutions, suspensions, emulsions, creams, gels, ointments, powders, liniments, salves, aerosols, transdermal patches, etc., which are, if desired, sterilised or mixed with auxiliary agents, e.g. preservatives, stabilisers, emulsifiers, wetting agents, fragrances, colouring agents, odour controllers, thickeners such as natural gums, etc. Particularly preferred topical compositions include ointments, creams or gels.

Ointments generally are prepared using either (1) an oleaginous base, i.e., one consisting of fixed oils or hydrocarbons, such as white petroleum, mineral oil, or (2) an absorbent base, i.e., one consisting of an anhydrous substance or substances which can absorb water, for example anhydrous lanolin. Customarily, following formation of the base, whether oleaginous or absorbent, the cannabinoids are added to an amount affording the desired concentration.

Creams are oil/water emulsions. They consist of an oil phase (internal phase), comprising typically fixed oils, hydrocarbons and the like, waxes, petroleum, mineral oil and the like and an aqueous phase (continuous phase), comprising water and any water-soluble substances, such as added salts. The two phases are stabilised by use of an emulsifying agent, for example, a surface active agent, such as sodium lauryl sulfite; hydrophilic colloids, such as acacia colloidal clays, veegum and the like. Upon formation of the emulsion, the cannabinoids can be added in an amount to achieve the desired concentration.

Gels comprise a base selected from an oleaginous base, water, or an emulsion-suspension base. To the base is added a gelling agent that forms a matrix in the base, increasing its viscosity. Examples of gelling agents are hydroxypropyl cellulose, acrylic acid polymers and the like. Customarily, the cannabinoid and the compound that removes or substantially removes or reduces the integrity of the outer membrane of a bacterium are added to the composition at the desired concentration at a point preceding addition of the gelling agent.

The amount of cannabinoid and a compound that removes or substantially removes or reduces the integrity of the outer membrane of a bacteria incorporated into a topical composition is not critical; the concentration should be within a range sufficient to permit ready application of the composition such that an effective amount of the cannabinoids is delivered.

### Ocular Compositions

Compositions of the invention may be administered via ocular delivery. Preferably, the ocular composition comprise a cannabinoid and a compound that removes or substantially removes or reduces the integrity of the outer membrane of a bacteria.

Ocular delivery encompasses delivery to the sclera, retina, intraocular fluid, tissue surrounding the eyeball. For example, the delivery may be via injection, topical delivery (creams, gels, ointments, sprays, eye drops), intraocular implant or other means.

Artificial tear vehicles may be used for ocular cannabinoid delivery. More viscous artificial tears use high concentrations of viscosity enhancing agents, such as Celluvisc®, high viscosity carboxymethyl cellulose (CMC) and Refresh Liquigel®, a blend of 0.35% high viscosity CMC and 0.65% low viscosity CMC.

Gelling agents may be used for cannabinoid delivery. Such agents may be instilled as liquid and then almost immediately triggered to a gel phase. Timoptic gel (gellan gum), AzaSite® (polycarbophil, poloxamer), and Besivance®, (polycarbophil, poloxamer), 0.3% alginate Keltrol® are examples of such agents. Another gelling agent is polycarbophil-poloxamer gels (eg Durasite®).

Ocular delivery may also comprise injecting the cannabinoid and the compound that removes or substantially removes or reduces the integrity of the outer membrane of a bacterium into the sclera, intraocular space or into the area behind the eye. Compositions suitable for ocular injection optionally include sterile aqueous solutions (where water-soluble) or dispersions and sterile powders for the extemporaneous preparation of sterile injectable solutions or dispersion. Alternatively, the therapeutic agents (e.g. a cannabinoid and a compound that removes or substantially removes or reduces the integrity of the outer membrane of a bacteria) are, in certain aspects encapsulated in liposomes and delivered in injectable solutions to assist their transport across cell membrane. Alternatively, or in addition, such preparations contain constituents of self-assembling pore structures to facilitate transport across the cellular membrane. The carrier, in various aspects, is a solvent or dispersion medium containing, for example, water, ethanol, polyol (for example, glycerol, propylene glycol and liquid polyethylene glycol, and the like), suitable mixtures thereof, and vegetable oils. Proper fluidity is maintained, for example and without limitation, by the use of a coating such as lecithin, by the maintenance of the required particle size in the case of dispersion and by the use of surfactants. Prolonged absorption of the injectable compositions is in certain aspects brought about by the use in the compositions of agents delaying absorption, for example, aluminium monostearate and gelatine.

### Oral Compositions

Compositions of the invention may be administered orally. Preferably, the oral composition comprises a cannabinoid and a compound that removes or substantially removes or reduces the integrity of the outer membrane of a bacterium.

Contemplated for use herein are oral solid dosage forms, which are described generally in Martin, Remington's Pharmaceutical Sciences, 18th Ed. (1990 Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatised with various polymers (E.g., U.S. Pat. No. 5,013,556). A description of possible solid dosage forms for the therapeutic is given by Marshall, in Modern Pharmaceutics, Chapter 10, Banker and Rhodes ed., (1979), herein incorporated by reference. In general, the composition will include therapeutic agents (e.g. a cannabinoid and a compound that removes or substantially removes or reduces the integrity of the outer membrane of a bacteria), and inert ingredients which allow for protection against the stomach environment, and release of the cannabinoid and the compound that removes or substantially removes or reduces the integrity of the outer membrane of a bacteria in the intestine.

For the cannabinoid and the compound that removes or substantially removes or reduces the integrity of the outer membrane of a bacteria of the invention, the location of release may be the stomach, the small intestine (the duodenum, the jejunum, or the ileum), or the large intestine. One skilled in the art has available compositions that will not dissolve in the stomach, yet will release the material in the duodenum or elsewhere in the intestine. In one aspect, the release will avoid the deleterious effects of the stomach environment, either by protection of the composition or by release of the cannabinoid and the compound that removes or substantially removes or reduces the integrity of the outer membrane of a bacteria beyond the stomach environment, such as in the intestine.

It is believed that the oral bioavailability of cannabinoids is only 4% to 12% and absorption is highly variable. Although most cannabinoids are generally easily absorbed due to their high partition coefficient (P), they are subject to degradation in the stomach and significant first-pass metabolism.

Preferably, the cannabinoid and the compound that removes or substantially removes or reduces the integrity of the outer membrane of a bacterium is released in the lower gastrointestinal tract.

The oral dosage method may be provided using an oral sustained release pharmaceutical composition comprising a therapeutically effective pharmaceutical composition according to the invention, and a release retardant.

In one aspect of the present invention the release retardant is a water-soluble, water swellable and/or water insoluble polymer. In particular, water-soluble polymers are selected from the group comprising are ethylcellulose, hydroxypropyl cellulose, hydroxypropyl methyl cellulose, an enteric coating; and a semipermeable membrane. In another aspect of the invention the release retardant is a non-polymeric release retardant. More particularly, the non-polymeric release retardant is hydrogenated castor oil. The compositions of the invention may be milled or granulated and compressed into tablets or encapsulated into capsules according to conventional procedures known in the art.

To ensure full gastric resistance, a coating impermeable to at least pH 5.0 is used. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This includes without limitation sugar coatings, or coatings that make the tablet easier to swallow. Exemplary capsules consist of a hard shell (such as gelatin) for delivery of dry therapeutic i.e. powder; for liquid forms, a soft gelatine shell may be used. The shell material of cachets in certain aspects is thick starch or other edible paper. For pills, lozenges, moulded tablets or tablet triturates, moist massing techniques are also contemplated, without limitation.

As used herein, the term “sustained release” means the gradual but continuous or sustained release over a relatively extended period of the therapeutic compound content after oral ingestion. The release may continue after the pharmaceutical composition has passed from the stomach and through until and after the pharmaceutical composition reaches the intestine. The phrase “sustained release” also means delayed release wherein release of the therapeutic compound is not immediately initiated upon the pharmaceutical composition reaching the stomach but rather is delayed for a period of time, for example, until when the pharmaceutical composition reaches the intestine. Upon reaching the intestine, the increase in pH may then trigger release of the therapeutic compound from the pharmaceutical composition.

Though term “release retardant” is used herein, means a substance that reduces the rate of release of a therapeutic compound from a pharmaceutical composition when orally ingested. The release retardant may be a polymer or a non-polymer. The release retardant may be used according to any one of several sustained release systems including, for example, a diffusion system, a dissolution system and/or an osmotic system.

In certain aspects, the therapeutic agent (e.g. a cannabinoid and a compound that removes or substantially removes or reduces the integrity of the outer membrane of a bacteria) is included in the composition as fine multi-particulates in the form of granules or pellets of particle size about 1 mm. The composition of the material for capsule administration is, in certain aspects, a powder, lightly compressed plug, or even as a tablet. In one aspect, the therapeutic agent (e.g. a cannabinoid and a compound that removes or substantially removes or reduces the integrity of the outer membrane of a bacteria) could be prepared by compression.

Colourants and flavouring agents may optionally be included. For example, compositions may be formulated (such as, and without limitation, by liposome or microsphere encapsulation) and then further contained within an edible product, such as a refrigerated beverage containing colorants and flavouring agents.

The volume of the composition may be diluted or increased with an inert material. These diluents could include carbohydrates, especially mannitol, alpha-lactose, anhydrous lactose, cellulose, sucrose, modified dextrans and starch. Certain inorganic salts are also optionally used as fillers including calcium triphosphate, magnesium carbonate and sodium chloride. Some commercially available diluents are Fast-Flo, Emdex, STA-Rx 1500, Emcompress and Avicell.

In other embodiments, disintegrants are included in the solid dosage form compositions of the present invention. Materials used as disintegrants include but are not limited to starch including the commercial disintegrant based on starch, Explotab. Sodium starch glycolate, Amberlite, sodium carboxymethylcellulose, ultramylopectin, sodium alginate, gelatine, orange peel, acid carboxymethyl cellulose, natural sponge and bentonite are also contemplated. Another form of the disintegrants is the insoluble cationic exchange resins. Powdered gums are also optionally used as disintegrants and as binders and these include, without limitation, powdered gums such as agar, Karaya or tragacanth. Alginic acid and its sodium salt are also useful as disintegrants.

Binders are contemplated to hold the cannabinoid and the compound that removes or substantially removes or reduces the integrity of the outer membrane of a bacteria together to form a hard tablet and include, without limitation, materials from natural products such as acacia, tragacanth, starch and gelatine. Other binders include, without limitation, methylcellulose (MC), ethyl cellulose (EC) and carboxymethyl cellulose (CMC). Polyvinyl pyrrolidone (PVP) and hydroxypropylmethyl cellulose (HPMC) are contemplated for use in alcoholic solutions to granulate the therapeutic.

An antifrictional agent may be optionally included in the compositions of the invention to prevent sticking during the composition process. Lubricants may be optionally used as a layer between the therapeutic and the die wall, and these can include but are not limited to: stearic acid including its magnesium and calcium salts, polytetrafluoroethylene (PTFE), liquid paraffin, vegetable oils and waxes. Exemplary soluble lubricants may also be used such as include sodium lauryl sulfate, magnesium lauryl sulfate, polyethylene glycol of various molecular weights, and Carbowax 4000 and 6000.

Glidants that might improve the flow properties of the compound during composition and to aid rearrangement during compression might be optionally added. The glidants may include without limitation starch, talc, pyrogenic silica and hydrated silicoaluminate.

To aid dissolution of the therapeutic agent (e.g. a cannabinoid and a compound that removes or substantially removes or reduces the integrity of the outer membrane of a bacteria) into the aqueous environment, a surfactant might be added in certain embodiments as a wetting agent. Surfactants may include, for example and without limitation, anionic detergents such as sodium lauryl sulfate, dioctyl sodium sulfosuccinate and dioctyl sodium sulfonate. Cationic detergents might be optionally used and could include, without limitation, benzalkonium chloride or benzethomium chloride. The list of potential nonionic detergents that could be included in the composition as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. When used, these surfactants could be present in the composition of the therapeutic agent either alone or as a mixture in different ratios.

Additives which that potentially enhance uptake of the therapeutic agent include, without limitation, the fatty acids oleic acid, linoleic acid and linolenic acid.

Controlled release composition may be desirable. In certain aspects, the therapeutic agents could be incorporated into an inert matrix that permits release by either diffusion or leaching mechanisms i.e., gums. In some aspects, slowly degenerating matrices may also be incorporated into the composition. Another form of a controlled release of this therapeutic is by a method based on the Oros therapeutic system (Alza Corp.), i.e. the drug is enclosed in a semipermeable membrane which allows water to enter and push drug out through a single small opening due to osmotic effects. Some enteric coatings also have a delayed release effect.

In other aspects, a mix of materials might be used to provide the optimum film coating. Film coating may be carried out, for example, in a pan coater or in a fluidized bed or by compression coating.

### Injectable Compositions

Compositions of the invention may be administered via injection. Preferably, the injectable composition comprise a cannabinoid and a compound that removes or substantially removes or reduces the integrity of the outer membrane of a bacteria.

The compositions suitable for injectable use optionally include sterile aqueous solutions (where water-soluble) or dispersions and sterile powders for the extemporaneous preparation of sterile injectable solutions or dispersion. Alternatively, the therapeutic agents of the invention are, in certain aspects encapsulated in liposomes and delivered in injectable solutions to assist their transport across cell membrane. Alternatively or in addition such preparations contain constituents of self-assembling pore structures to facilitate transport across the cellular membrane. The carrier, in various aspects, is a solvent or dispersion medium containing, for example, water, ethanol, polyol (for example, glycerol, propylene glycol and liquid polyethylene glycol, and the like), suitable mixtures thereof, and vegetable oils. Proper fluidity is maintained, for example and without limitation, by the use of a coating such as lecithin, by the maintenance of the required particle size in the case of dispersion and by the use of surfactants. Prolonged absorption of the injectable compositions is in certain aspects brought about by the use in the compositions of agents delaying absorption, for example, aluminum monostearate and gelatin.

The invention also provides an injectable sustained release pharmaceutical composition comprising a therapeutically effective pharmaceutical composition according to the invention, and a release retardant. The release retardant may be, for example, aluminum monostearate and gelatin.

Sterile injectable solutions are prepared by incorporating the therapeutic agents in the required amount in an appropriate solvent with various of the other ingredients enumerated above, as required, followed by filtered sterilisation. Generally, dispersions are prepared by incorporating the various sterilised therapeutic agents into a sterile vehicle that contains the basic dispersion medium and the required other ingredients from those enumerated above. In the case of sterile powders for the preparation of sterile injectable solutions, preparation in certain aspects include without limitation vacuum drying and freeze-drying techniques that yield a powder of the therapeutic agents plus any additional desired ingredient from previously sterile-filtered solution thereof.

### Nasal and Pulmonary Compositions

Compositions of the invention may be administered via nasal or pulmonary delivery. Preferably, the nasal or pulmonary composition comprise a cannabinoid and a compound that removes or substantially removes or reduces the integrity of the outer membrane of a bacterium.

A wide range of mechanical devices designed for pulmonary delivery of therapeutic agents exist, including but not limited to nebulizers, metered-dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass.

All such devices require the use of compositions suitable for the dispensing of the cannabinoid and the compound that removes or substantially removes or reduces the integrity of the outer membrane of a bacterium. Typically, each composition is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated.

Compositions suitable for use with a nebulizer, either jet or ultrasonic, will typically comprise the cannabinoid and the compound that removes or substantially removes or reduces the integrity of the outer membrane of a bacterium suspended in water or non-aqueous solvent. The composition may also include a buffer and a simple sugar (e.g., for stabilization and regulation of osmotic pressure). The nebulizer composition may also contain a surfactant, to reduce or prevent surface induced aggregation of the cannabinoid and the compound that removes or substantially removes or reduces the integrity of the outer membrane of a bacteria caused by atomization of the solution in forming the aerosol.

Compositions for use with a metered dose inhaler device will generally comprise a finely divided powder containing the cannabinoid and the compound that removes or substantially removes or reduces the integrity of the outer membrane of a bacteria suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2 tetrafluoroethane, or combinations thereof. Suitable surfactants include sorbitan trioleate and soya lecithin. Oleic acid may also be useful as a surfactant.

Compositions for dispensing from a powder inhaler device will comprise a finely divided dry powder containing the cannabinoid and the compound that removes or substantially removes or reduces the integrity of the outer membrane of a bacteria and may also include a bulking agent, such as lactose, sorbitol, sucrose, or mannitol in amounts which facilitate dispersal of the powder from the device, e.g., 50 to 90% by weight of the composition. The cannabinoid and the compound that removes or substantially removes or reduces the integrity of the outer membrane of a bacteria should most advantageously be prepared in particulate form with an average particle size of less than 10 microns, most preferably 0.5 to 5 microns, for most effective delivery to the distal lung.

Nasal delivery of a cannabinoid and a compound that removes or substantially removes or reduces the integrity of the outer membrane of a bacterium in the treatment methods of the present invention is also contemplated. Nasal delivery allows the passage of the cannabinoid and the compound that removes or substantially removes or reduces the integrity of the outer membrane of a bacteria to the blood stream directly after administering the therapeutic product to the nose, without the necessity for deposition of the cannabinoid and the compound that removes or substantially removes or reduces the integrity of the outer membrane of a bacteria in the lung. Compositions for nasal delivery include those with dextran or cyclodextran.

### Kits

The invention also provides kits for use in the instant methods. Kits of the invention include one or more containers comprising a cannabinoid and a compound that removes or substantially removes or reduces the integrity of the outer membrane of a bacterium, and instructions for use in accordance with any one of the methods described herein.

The kit may further comprise a description of selecting an individual suitable for treatment based on identifying whether that individual has an infection by a bacterium. The kit may further comprise a description of administering a cannabinoid and a compound that removes or substantially removes or reduces the integrity of the outer membrane of a bacterium as described herein to an individual at risk of developing an infection by a bacterium.

The instructions generally include information as to dosage, dosing schedule, and route of administration for the intended treatment. The containers may be unit doses, bulk packages (e.g. multi0dose packages) or sub-unit doses. Instructions supplied in the kits of the invention are typically written instructions on a label or package insert. The label or package insert indicates that the composition is used for treating, ameliorating and/or preventing an infection by a Gram-negative bacterium. Instructions may be provided for practising any of the methods described herein.

### General

Those skilled in the art will appreciate that the invention described herein is susceptible to variations and modifications other than those specifically described. The invention includes all such variation and modifications. The invention also includes all of the steps, features, compositions and therapeutic agents referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, compositions and methods are clearly within the scope of the invention as described herein.

The invention described herein may include one or more range of values (eg. Size, displacement and field strength etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” or “therapeutic agent” may mean one active agent or therapeutic agent, or may encompass two or more active agents or therapeutic agents.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

Further features of the present invention are more fully described in the following non-limiting Examples. This description is included solely for the purposes of exemplifying the present invention. It should not be understood as a restriction on the broad description of the invention as set out above.

### Example 1

### Cell-Wall Disruption Studies of Cannabidiol Against Wild-Type Gram-Negative Bacteria

The experiment was performed to assess the in vitro activity of cell-wall disrupting agent Polymyxin B in combination with cannabidiol against wild type species of Gram-negative bacteria.

Cannabidiol was tested in combination with Polymyxin B. DMSO alone was used as a control. The compounds were provided as stock solutions of 1.28 mg/mL in DMSO. The compounds were stored short term at 4° C.

A positive control of just bacteria and a negative control of only media was included for every plate tested. All positive inhibitor controls were within expected ranges across strains. A negative media control containing cannabidiol was used to assess the effects of the insolubility of the compound on the optical density.

### Minimum Inhibitory Concentration (MIC) Micro-Broth Dilution Assay

Bacteria were cultured in Cation-adjusted Mueller Hinton broth (CaMHB; BD, Cat. No. 212322) at 37° C. overnight, then diluted 40-fold and incubated at 37° C. for a further 2-3 h. The resultant mid-log phase cultures were diluted in CaMHB and 45 μl was added to wells of compound-containing 384-well plates to give a final cell density of 5×10⁵ CFU/mL. The plates were covered and incubated at 37° C. for 18-20 h.

### MIC Detection and Analysis

Optical density was read at 600 nm (OD600) using Tecan M1000 Pro Spectrophotometer. MIC was determined as the lowest concentration at which OD600 demonstrated ≥90% growth inhibition compared to growth control. Analysis was performed using Microsoft Excel.

The synergistic effect was calculated based on the fractional inhibitory concentration; synergy is seen when the FICI≤0.5:

$$FICI = \frac{MIC cpdA in combination}{MIC cpdA alone} + \frac{MIC cpdB in combination}{MIC cpdB alone}$$

Final FICI scores were an average of all combinations seen to give a robust FICI score across the active combinations.

TABLE 1
Tested cell-wall disrupting agents and compounds

| | | | | Min |
|---|---|---|---|---|
| | Compound | Stock conc | Max test | test conc |
| MCC | name | (mg/mL) | conc (μg/mL) | (μg/mL) |
| MCC_000636 | Polymyxin B | 1.28 | 32 | 0.0009 |
| MCC_009427 | Cannabidiol | 1.28 | 32 | 0.0009 |

TABLE 2
Tested Bacteria

| ID | Species | Strain | Description |
|---|---|---|---|
| GN_001 | Escherichia coli | ATCC 25922 | Control, FDA |
| | | | strain Seattle |
| | | | 1946 |
| GN_034 | Acinetobacter baumannii | ATCC 19606 | Control, Type |
| | | | strain |
| GN_042 | Pseudomonas aeruginosa | ATCC 27853 | Control strain |

Cannabidiol alone is inactive against the Gram-negative bacteria (MIC>256 μg/mL). Cannabidiol in combination with Polymyxin B (MCC_00636) was effective against every strain tested.

TABLE 3
Minimal Inhibitory Concentration (MIC)

| | GN_001 Escherichia coli | | | | GN_042 Pseudomonas aeruginosa | | | |
|---|---|---|---|---|---|---|---|---|
| Cannabidiol | >256 | >256 | >256 | >256 | >256 | >256 | >256 | >256 |
| MCC_000636 | nt | nt | 1 | 0.5 | nt | nt | 1 | 1 |

| | GN_034 Acinetobacter baumannii | | | |
|---|---|---|---|---|
| Cannabidiol | >256 | >256 | >256 | >256 |
| MCC_000636 | nt | nt | 1 | 0.5 |

nt . . . Not tested

TABLE 4
Synergy - Average FICI score

| | |
|---|---|
| FICI* | Synergy |

GN_001 Escherichia coli

| | | |
|---|---|---|
| Cannabidiol | 0.15 | YES |
| MCC_006636 | | |

GN_034 Acinetobacter baumannii

| | | |
|---|---|---|
| Cannabidiol | 0.135 | YES |
| MCC_006636 | | |

GN_042 Pseudomonas aeruginosa

| | | |
|---|---|---|
| Cannabidiol | 0.265 | YES |
| MCC_006636 | | |

### Example 2

### Cell-Wall Disruption Studies of Cannabidiol Against Wild-Type Gram-Negative Bacteria

The experiment was conducted to investigate the in vitro activity of the cell-wall disrupting agent cefuroxime in combination with cannabidiol, against wild type species of Gram-negative bacteria.

Methods were as for Example 1. Cannabidiol was tested in combination with cefuroxime.

TABLE 5
Tested cell-wall disrupting agents and compounds

| | | | Max test | |
|---|---|---|---|---|
| | Compound | Stock conc | conc | Min test conc |
| MCC | name | (mg/mL) | (ng/mL) | (ng/mL) |
| MCC_009386 | Cefuroxime | 10 | 256 | 0.0078 |
| MCC_009427 | Cannabidiol | 10 | 256 | 0.0078 |

TABLE 6
Tested bacteria

| ID | Species | Strain | Description |
|---|---|---|---|
| GN_034:02 | Acinetobacter baumannii | ATCC 19606 | Control, |
| | | | Type strain |

Cannabidiol combined with cefuroxime is effective against Acinetobacter baumannii.

TABLE 7
Minimum Inhibitory Concentration (MIC)

| | GN_034 Acinetobacter baumannii | | | |
|---|---|---|---|---|
| MCC_009427 | >256 | >256 | >256 | >256 |
| MCC_009386 | >32 | >32 | 64 | 64 |

TABLE 8
Synergy - Average FICI score

| | | CBD MIC (μg/mL) with | | | | | | |
|---|---|---|---|---|---|---|---|---|
| | MIC of | Cefuroxime at concentration (μg/mL) | | | | | | FICI |
| Strain | cefuroxime | 4 | 8 | 16 | 32 | 64 | 128 | range |
| GN_034 | 64 | >256 | >256 | 32 | 16 | 0.0625 | 0.00097656 | 0.38-2 |
| | 64 | >256 | >256 | 32 | 32 | 0.0625 | 0.00097656 | 0.38-2 |

*FICI scores were calculated using an MIC of 256 μg/mL for cannabidiol.

### Example 3

### Cell-Wall Disruption Studies of Cannabidiol Against Wild-Type Gram-Negative Bacteria

The experiment was conducted to investigate the in vitro activity of colistin in combination with cannabidiol, against wild type species of Gram-negative bacteria.

Methods were as for Example 1. Cannabidiol was tested in combination with colistin (MCC_00094).

TABLE 9
Tested cell-wall disrupting agents and compounds

| | | | Max | |
|---|---|---|---|---|
| | Compound | Stock conc | test conc | Min test conc |
| MCC | name | (mg/mL) | (μg/mL) | (μg/mL) |
| MCC_000094 | Colistin | 10 | 16 | 0.00049 |
| MCC_009427 | Cannabidiol | 10 | 256 | 0.0078 |

TABLE 10
Tested bacteria

| ID | Species | Strain | Description |
|---|---|---|---|
| GN_001 | Escherichia coli | ATCC 25922 | Control, FDA strain |
| | | | Seattle 1946 |
| GN_034 | Acinetobacter | ATCC 19606 | Control, Type strain |
| | baumannii | | |

Cannabidiol combined with colistin is effective against Escherichia coli and Acinetobacter baumannii.

TABLE 11
Minimum Inhibitory Concentration (MIC)

| | GN_001 Escherichia coli | | | |
|---|---|---|---|---|
| Cannabidiol | >256 | >256 | >256 | >256 |
| MCC_000094 | 0.25 | 0.5 | 2 | 2 |
| | GN_034 Acinetobacter baumannii | | | |
| Cannabidiol | >256 | >256 | >256 | >256 |
| MCC_000094 | 1 | 1 | 1 | 1 |

TABLE 12
Synergy - Average FICI scores

| | | CBD MIC (μg/mL) with | | | | | | |
|---|---|---|---|---|---|---|---|---|
| | MIC of | Colistin at concentration (μg/mL) | | | | | | FICI |
| Strain | colistin | 0.015625 | 0.03125 | 0.0625 | 0.125 | 0.25 | 0.5 | range |
| GN_001 | 0.5 | >256 | >256 | 8 | 2 | 1 | 0.125 | 0.16-1.06 |
| | 0.5 | >256 | >256 | 8 | 2 | 1 | 0.125 | 0.16-1.06 |
| | MIC of | | | | | | | |
| Strain | colistin | 0.03125 | 0.0625 | 0.125 | 0.25 | 0.5 | 1 | |
| GN_034 | 1 | 4 | 2 | 1 | 1 | 0.25 | 0.00097656 | 0.05-1 |
| | 1 | 4 | 2 | 1 | 1 | 1 | 0.00097656 | 0.05-1 |

*FICI scores were calculated using an MIC of 256 μg/mL for cannabidiol.

### Example 4

### Cell-Wall Disruption Studies of Cannabidiol Against Wild-Type Gram-Negative Bacteria

The experiment was conducted to investigate the in vitro activity of octapeptin MCC_006442 in combination with cannabidiol, against wild type Gram-negative bacteria.

Methods were as for Example 1. Cannabidiol was tested in combination with octapeptin MCC_006442.

TABLE 13
Tested cell-wall disrupting agents and compounds

| | | Stock | Max | |
|---|---|---|---|---|
| | Compound | conc | test conc | Min test conc |
| MCC | name | (mg/mL) | (ng/mL) | (ng/mL) |
| MCC_006442 | octapeptin | 10 | 128 | 0.00390625 |
| MCC_009427 | Cannabidiol | 10 | 256 | 0.0078 |

TABLE 14
Tested bacteria

| ID | Species | Strain | Description |
|---|---|---|---|
| GN_003 | Klebsiella pneumoniae | ATCC 13883 | Type, Polymyxin |
| | | | heteroresistant |
| GN_034 | Acinetobacter baumannii | ATCC 19606 | Control, Type |
| | | | strain |

Cannabidiol alone is inactive against the Gram-negative bacteria (MIC>256 μg/mL). Cannabidiol combined with octapeptin MCC_006442 is effective against Klebsiella pneumoniae and Acinetobacter baumannii.

TABLE 15
Minimum Inhibitory Concentration (MIC)

| | GN_034 Acinetobacter baumannii | | | | GN_003 Klebsiella pneumoniae | | | |
|---|---|---|---|---|---|---|---|---|
| Cannabidiol | >256 | >256 | >256 | >256 | >256 | >256 | >256 | >256 |
| MCC_006442 | 32 | 32 | 32 | 32 | 8 | 8 | 8 | 16 |

TABLE 16
Average FICI scores

| | | CBD MIC (μg/mL) with | | | | | | |
|---|---|---|---|---|---|---|---|---|
| | MIC of | octapeptin MCC_006442 at concentration (μg/mL) | | | | | | FICI |
| Strain | MCC_006442 | 0.125 | 0.25 | 0.5 | 1 | 2 | 4 | range |
| GN_003 | 8 | >256 | >256 | >256 | >256 | 1 | 0.25 | 0.25-1.13 |
| | 16 | >256 | >256 | >256 | >256 | 1 | 0.25 | 0.13-1.06 |
| GN_034 | 32 | >256 | >256 | >256 | 16 | 1 | 0.5 | 0.07-1.02 |
| | 32 | >256 | >256 | >256 | 16 | 1 | 0.5 | 0.07-1.02 |

*FICI scores were calculated using an MIC of 256 μg/mL for cannabidiol.

### Example 5

### Cell-Wall Disruption Studies of Cannabidiol Against Wild-Type Gram-Negative Bacteria

The experiment was conducted to investigate the in vitro activity of cell-wall disrupting agents aztreonam and cefepime in combination with cannabidiol, against wild type Gram-negative bacteria.

Methods were as for Example 1. Cannabidiol was tested in combination with aztreonam and cefepime.

TABLE 17
Tested cell-wall disrupting agents and compounds

| | | Stock | Max | |
|---|---|---|---|---|
| | Compound | conc | test conc | Min test conc |
| MCC | name | (mg/mL) | (μg/mL) | (μg/mL) |
| MCC_007251 | Aztreonam | 10 | 128 | 0.00390625 |
| MCC_007306 | Cefepime | 10 | 256 | 0.0078125 |
| MCC_009427 | Cannabidiol | 10 | 256 | 0.0078 |

TABLE 18
Tested bacteria

| ID | Species | Strain | Description |
|---|---|---|---|
| GN_034 | Acinetobacter baumannii | ATCC 19606 | Control, Type |
| | | | strain |

Cannabidiol combined with aztreonam or cefepime is effective against Acinetobacter baumannii.

TABLE 19
Minimum Inhibitory Concentration (MIC)

| | GN_034 Acinetobacter baumannii | | | |
|---|---|---|---|---|
| Cannabidiol | >256 | >256 | >256 | >256 |
| MCC_007251 | 16 | 16 | nt | nt |
| MCC_007306 | 16 | 16 | nt | nt |

nt . . . Not tested

TABLE 20
Synergy - Average FICI scores

| | | CBD MIC (μg/mL) with | | | | | | |
|---|---|---|---|---|---|---|---|---|
| | MIC of | Aztreonam at concentration (μg/mL) | | | | | | FICI |
| Strain | aztreonam | 0.5 | 1 | 2 | 4 | 8 | 16 | range |
| GN_034 | 32 | >256 | >256 | >256 | 32 | 32 | 0.00097656 | 0.38-1.13 |
| | 32 | >256 | >256 | >256 | 32 | 32 | 0.00097656 | 0.38-1.13 |

| | | CBD MIC (μg/mL) with | | | | | | |
|---|---|---|---|---|---|---|---|---|
| | MIC of | Cefepime at concentration (μg/mL) | | | | | | FICI |
| Strain | Cefepime | 0.25 | 0.5 | 1 | 2 | 4 | 8 | range |
| GN_034 | 16 | >256 | >256 | >256 | >256 | 32 | 16 | 0.38-1.13 |
| | 16 | >256 | >256 | >256 | >256 | 32 | 16 | 0.38-1.13 |

*FICI scores were calculated using an MIC of 256 μg/mL for cannabidiol.

### Example 6

### Cell-Wall Disruption Studies of Cannabidiol Against Wild-Type Gram-Negative Bacteria

The experiment was conducted to investigate the in vitro activity of cell-wall disrupting agent Polymyxin B in combination with cannabidiol, against wild type Gram-negative bacteria.

Methods were as for Example 1. Cannabidiol was tested in combination with Polymyxin B.

TABLE 21
Tested cell-wall disrupting agents and compounds

| | | Stock | Max | |
|---|---|---|---|---|
| | Compound | conc | test conc | Min test conc |
| MCC | name | (mg/mL) | (ng/mL) | (ng/mL) |
| MCC_000636 | Polymyxin B | 10 | 16 | 0.00049 |
| MCC_009427 | Cannabidiol | 10 | 256 | 0.0078 |

TABLE 22
Tested bacteria

| ID | Species | Strain | Description |
|---|---|---|---|
| GN_001:02 | Escherichia coli | ATCC 25922 | Control, FDA strain |
| | | | Seattle 1946 |
| GN_034:02 | Acinetobacter | ATCC 19606 | Control, Type strain |
| | baumannii | | |
| GN_042:02 | Pseudomonas | ATCC 27853 | Control strain |
| | aeruginosa | | |

Cannabidiol combined with Polymyxin B is effective against Escherichia coli, Acinetobacter baumannii and Pseudomonas aeruginosa.

TABLE 23
Minimum Inhibitory Concentration (MIC)

| | GN_001 Escherichia coli | | | | GN_042 Pseudomonas aeruginosa | | | |
|---|---|---|---|---|---|---|---|---|
| Cannabidiol | >256 | >256 | >256 | >256 | >256 | >256 | >256 | >256 |
| MCC_000636 | 0.25 | 0.25 | 0.5 | 1 | 0.5 | 0.5 | 2 | 2 |

| | GN_034 Acinetobacter baumannii | | | |
|---|---|---|---|---|
| Cannabidiol | >256 | >256 | >256 | >256 |
| MCC_000636 | 0.25 | 0.125 | 2 | 1 |

TABLE 24
Synergy - Average FICI scores

| | | CBD MIC (μg/mL) with | | | | | | |
|---|---|---|---|---|---|---|---|---|
| | MIC of | Polymyxin B at concentration (μg/mL) of | | | | | | FICI |
| Strain | Polymyxin B | 0.015625 | 0.03125 | 0.0625 | 0.125 | 0.25 | 0.5 | range |
| GN_001 | 1 | >256 | >256 | >256 | >256 | 2 | 0.00097656 | 0.26-1.13 |
| | 1 | >256 | >256 | >256 | >256 | 4 | 0.00097656 | 0.27-1.13 |
| GN_034 | 2 | >256 | >256 | >256 | 8 | 4 | 0.00097656 | 0.09-1.03 |
| | 1 | >256 | >256 | >256 | 16 | 8 | 0.00097656 | 0.19-1.06 |
| GN_042 | 2 | >256 | >256 | >256 | >256 | >256 | 2 | 0.26-1.13 |
| | 2 | >256 | >256 | >256 | >256 | >256 | 4 | 0.27-1.13 |

*FICI scores were calculated using an MIC of 256 μg/mL for cannabidiol.

### Example 7

### Cell-Wall Disruption Studies of Cannabidiol Against Wild-Type Gram-Negative Bacteria

The experiment was performed to assess the in vitro activity of cell-wall disrupting agent octapeptin MCC_006442 in combination with cannabidiol against wild type species of Gram-negative bacteria.

Methods were as for Example 1. Cannabidiol was tested in combination with octapeptin MCC_006442.

TABLE 25
Tested cell-wall disrupting agents and compounds

| | | | Stock | Max test | Min test |
|---|---|---|---|---|---|
| | | Compound | conc | conc | conc |
| MCC | Batch | name | (mg/mL) | (μg/mL) | (μg/mL) |
| MCC_006442 | 002 | octapeptin | 1.28 | 32 | 0.0009 |
| MCC_009427 | 002 | Cannabidiol | 1.28 | 32 | 0.0009 |

TABLE 26
Tested Bacteria

| ID | Species | Strain | Description |
|---|---|---|---|
| GN_001 | Escherichia | ATCC 25922 | Control, FDA strain |
| | coli | | Seattle 1946 |
| GN_004 | Klebsiella | ATCC 13883 | Type, Polymyxin |
| | pneumoniae | | heteroresistant |
| GN_034 | Acinetobacter | ATCC 19606 | Control, Type strain |
| | baumannii | | |

Cannabidiol in combination with octapeptin MCC_006442 was effective against Klebsiella pneumoniae and Acinetobacter baumannii, and slightly effective against Escherichia coll.

TABLE 27
Minimal Inhibitory Concentration (MIC)

| | GN_001 Escherichia coli | | | | GN_004 Klebsiella pneumoniae | | | |
|---|---|---|---|---|---|---|---|---|
| Cannabidiol | >256 | >256 | >256 | >256 | >256 | >256 | >256 | >256 |
| MCC_006442 | 2 | 4 | nt | nt | 4 | 4 | nt | nt |

| | GN_034 Acinetobacter baumannii | | | |
|---|---|---|---|---|
| Cannabidiol | >256 | >256 | >256 | >256 |
| MCC_006442 | 8 | 8 | nt | nt |

nt . . . Not tested

TABLE 28
Synergy - Average FICI score
GN_001 Escherichia coli

| | FICI* |
|---|---|
| Cannabidiol | 0.25 |
| MCC_006442 | |

GN_034 Acinetobacter baumannii

| | FICI* |
|---|---|
| Cannabidiol | 0.17 |
| MCC_006442 | |

GN_004 Klebsiella pneumoniae

| | FICI* |
|---|---|
| Cannabidiol | 0.27 |
| MCC_006442 | |

*FICI scores were calculated using an MIC of 256 μg/mL for cannabidiol

### Example 8

### Cell-Wall Disruption Studies of Cannabidiol Against Multidrug Resistant Gram-Negative Bacteria

The experiment was conducted to investigate the in vitro activity of cell-wall disrupting agent Polymyxin B in combination with cannabidiol, against multiple-drug resistant (MDRs) Gram-negative bacteria.

Methods were as for Example 1. Cannabidiol was tested in combination with Polymyxin B.

TABLE 29
Tested cell-wall disrupting agents and compounds

| | | Stock | Max test | |
|---|---|---|---|---|
| | Compound | conc | conc | Min test conc |
| MCC | name | (mg/mL) | (μg/mL) | (μg/mL) |
| MCC_00636 | Polymyxin B | 10 | 32 | 0.00097656 |
| MCC_009427 | Cannabidiol | 10 | 256 | 0.0078 |

TABLE 30
Tested bacteria

| ID | Species | Strain | Description |
|---|---|---|---|
| GN_016 | Enterobacter | CI Patercon BoxNo: 79 | MDR |
| | cloacae | | |
| GN_038 | Acinetobacter | 03.021.1 | Colistin resistant |
| | baumannii | | |
| GN_039 | Acinetobacter | 03.021.1 | Colistin resistant |
| | baumannii | | |
| GN_177 | Pseudomonas | FADDI-PA067 | Polymyxin resistant |
| | aeruginosa | | |

Cannabidiol combined with Polymyxin B displays synergy against Enterobacter cloacae, Acinetobacter baumannii, Acinetobacter baumannii, and Pseudomonas aeruginosa.

TABLE 31
Minimum Inhibitory Concentration (MIC)

| | GN_016 Enterobacter cloacae | | | | GN_038 Acinetobacter baumannii | | | |
|---|---|---|---|---|---|---|---|---|
| Cannabidiol | >256 | >256 | >256 | >256 | >256 | >256 | >256 | >256 |
| MCC_000636 | 16 | 8 | 8 | 8 | 0.125 | 0.125 | 0.25 | 0.125 |
| | GN_039 Acinetobacter baumannii | | | | GN_177 Pseudomonas aeruginosa | | | |
| Cannabidiol | >256 | >256 | >256 | >256 | >256 | >256 | >256 | >256 |
| MCC_000636 | 0.5 | 0.25 | 0.25 | 0.25 | 2 | 2 | 2 | 2 |

TABLE 32
Average FICI scores
CBD MIC (μg/mL) with MCC_00636 at concentration (μg/mL)

| | MIC of | | | | |
|---|---|---|---|---|---|
| Strain | MCC_00636 | 0.03125 | 0.0625 | 0.125 | 0.25 |
| GN_016 | 8 | >256 | >256 | >256 | 2 |
| | 8 | >256 | >256 | >256 | 2 |
| | | 0.00195313 | 0.00390625 | 0.0078125 | 0.015625 |
| GN_038 | 1 | >256 | >256 | >256 | >256 |
| | 1 | >256 | >256 | >256 | >256 |
| | | 0.00390625 | 0.0078125 | 0.015625 | 0.03125 |
| GN_039 | 1 | >256 | >256 | >256 | >256 |
| | 1 | >256 | >256 | >256 | >256 |
| | | 0.015625 | 0.03125 | 0.0625 | 0.125 |
| GN_177 | 2 | >256 | >256 | >256 | >256 |
| | 2 | >256 | >256 | >256 | >256 |

| | | | | | | FICI |
|---|---|---|---|---|---|---|
| Strain | 0.5 | 1 | 2 | 4 | 8 | range |
| GN_016 | 0.5 | 0.5 | 0.5 | 0.5 | 0.00097656 | 0.04-1.02 |
| | 1 | 1 | 1 | 1 | 0.00097656 | 0.04-1.02 |
| | 0.03125 | 0.0625 | 0.125 | 0.25 | 0.5 | |
| GN_038 | >256 | >256 | 32 | 2 | 0.00097656 | 0.25-1.06 |
| | >256 | >256 | 32 | 4 | 0.00097656 | 0.25-1.06 |
| | 0.0625 | 0.125 | 0.25 | 0.5 | 1 | |
| GN_039 | >256 | 4 | 4 | 0.00097656 | 0.00097656 | 0.14-1.06 |
| | >256 | 4 | 4 | 0.00097656 | 0.00097656 | 0.14-1.06 |
| | 0.25 | 0.5 | 1 | 2 | 4 | |
| GN_177 | >256 | 4 | 0.5 | 0.00097656 | 0.00097656 | 0.27-2 |
| | >256 | 4 | 0.5 | 0.00097656 | 0.00097656 | 0.27-2 |

*FICI scores were calculated using an MIC of 256 μg/mL for cannabidiol, MIC of 128 μg/mLfor MCC_00636 for GN_016, GN_177

### Example 9

### Cell-Wall Disruption Studies of Cannabidiol Against Multidrug Resistant Gram-Negative Bacteria

The experiment was conducted to investigate the in vitro activity of cell-wall disrupting agent MCC_09222 in combination with cannabidiol, against multiple-drug resistant (MDRs) Gram-negative bacteria.

Methods were as for Example 1. Cannabidiol was tested in combination with MCC_09222.

TABLE 33
Tested cell-wall disrupting agents and compounds

| | | Stock | Max test | |
|---|---|---|---|---|
| | Compound | conc | conc | Min test conc |
| MCC | name | (mg/mL) | (μg/mL) | (μg/mL) |
| MCC_09222 | MCC_09222 | 10 | 128 | 0.00390625 |
| MCC_009427 | Cannabidiol | 10 | 256 | 0.0078 |

TABLE 34
Tested bacteria

| ID | Species | Strain | Description |
|---|---|---|---|
| GN_016 | Enterobacter | CI Patercon BoxNo: 79 | MDR |
| | cloacae | | |
| GN_038 | Acinetobacter | 03.021.1 | Colistin resistant |
| | baumannii | | |
| GN_039 | Acinetobacter | 03.021.1 | Colistin resistant |
| | baumannii | | |
| GN_045 | Klebsiella | BAA-2146 | NDM-1 |
| | pneumoniae | | |
| GN_110 | Escherichia coli | HS71 | CTX-M |

Cannabidiol in combination with MCC_09222 showed synergistic effect against Enterobacter cloacae, Acinetobacter baumannii, Acinetobacter baumannii, Klebsiella pneumonia, and Escherichia coli.

TABLE 35
Minimum Inhibitory Concentration (MIC)

| | GN_016 Enterobacter cloacae | | | | GN_038 Acinetobacter baumannii | | | |
|---|---|---|---|---|---|---|---|---|
| MCC_009427 | >256 | >256 | >256 | >256 | >256 | >256 | >256 | >256 |
| MCC_09222 | >128 | >128 | >128 | >128 | 128 | 128 | 128 | 128 |
| MCC_000636 | 16 | 8 | 8 | 8 | 0.125 | 0.125 | 0.25 | 0.125 |

| | |
|---|---|
| GN_039 Acinetobacter baumannii | GN_045 Klebsiella pneumoniae |

| | | | | | | | | |
|---|---|---|---|---|---|---|---|---|
| MCC_009427 | >256 | >256 | >256 | >256 | >256 | >256 | >256 | >256 |
| MCC_09222 | 128 | 128 | 128 | 128 | 128 | >128 | 64 | 128 |
| MCC_000636 | 0.5 | 0.25 | 0.25 | 0.25 | 0.5 | 0.25 | 0.25 | 0.25 |

| | GN_110 Escherichia coli | | | |
|---|---|---|---|---|
| MCC_009427 | >256 | >256 | >256 | >256 |
| MCC_09222 | 32 | 32 | 64 | 32 |
| MCC_000636 | 0.0625 | 0.0625 | 0.0625 | 0.0625 |

TABLE 36
Average FICI scores
CBD MIC (μg/mL) with MCC_09222 at concentration (μg/mL)

| | MIC of | | | | | | | | | | |
|---|---|---|---|---|---|---|---|---|---|---|---|
| Strain | MCC_00631 | 0.5 | 1 | 2 | 4 | 8 | 16 | 32 | 64 | 128 | FICI range |
| GN_016 | >128 | >256 | >256 | >256 | >256 | 2 | 1 | 1 | 1 | 1 | 0.07-1.03 |
| | >128 | >256 | >256 | >256 | >256 | 2 | 2 | 1 | 1 | 1 | 0.07-1.03 |
| GN_038 | 128 | >256 | >256 | >256 | >256 | 2 | 2 | 0.5 | 0.00097656 | 0.00097656 | 0.07-1.03 |
| | 128 | >256 | >256 | >256 | >256 | 4 | 2 | 1 | 0.00097656 | 0.00097656 | 0.08-.03 |
| GN_039 | 128 | >256 | >256 | >256 | >256 | 1 | 1 | 0.5 | 0.00097656 | 0.00097656 | 0.07-1.03 |
| | 128 | >256 | >256 | >256 | >256 | 4 | 2 | 1 | 0.00097656 | 0.00097656 | 0.08-1.03 |
| GN_045 | 128 | >256 | >256 | >256 | >256 | 16 | 4 | 2 | 2 | 0.00097656 | 0.13-1.03 |
| | 128 | >256 | >256 | >256 | >256 | 16 | 2 | 2 | 2 | 0.00097656 | 0.13-1.03 |
| GN_110 | 32 | >256 | >256 | 4 | 1 | 1 | 0.5 | 0.00097656 | 0.00097656 | 0.00097656 | 0.08-4 |
| | 32 | >256 | >256 | 4 | 1 | 1 | 1 | 0.00097656 | 0.00097656 | 0.00097656 | 0.08-4 |

*FICI scores were calculated using an MIC of 256 μg/mL for cannabidiol, MIC of 128 μg/mL for MCC_00631 for GN_016, GN_177

### Example 10

Potentiation Studies of Cannabidiol with Polymyxin B Nonapeptide

The experiment was performed to assess whether the in vitro activity of cannabidiol (CBD) against Gram-negative bacteria could be improved in the presence of sub-MIC concentrations of Polymyxin B nonapeptide (PMBN) (MCC_9298), a cationic cyclic peptide derived from polymyxin B.

TABLE 37
Tested bacteria

| ID | Species | Strain | Description |
|---|---|---|---|
| GN_001 | Escherichia coli | ATCC 25922 | FDA control |
| GN_003 | Klebsiella | ATCC 700603 | ESBL, Resistant MDR |
| | quasipneumoniae | | |
| | subsp. | | |
| | similipneumoniae | | |
| GN_034 | Acinetobacter | ATCC 19606 | Type strain |
| | baumannii | | |
| GN_042 | Pseudomonas | ATCC 27853 | Control strain |
| | aeruginosa | | |

CBD was supplied sample as a dry solid by Botanix Pharmaceuticals. Stock solutions at 10 mg/mL in DMSO were prepared freshly on the day of experiment as stock solutions of 640 μg/mL. CBD and storage short term at 4° C. before and after the assay. Polymyxin nonapeptide (PMBN) was synthesised by WuXi AppTec.

### Minimum Inhibitory Concentration (MIC) Broth Microdilution

Bacteria (Table 37) were cultured in Cation-adjusted Mueller Hinton Broth CaMHB at 370 C overnight, then diluted 40-fold and incubated at 37° C. for another 2-3 hours. The inoculum was then diluted in CaMHB to give a mid-log phase cultures and 40 μL was added to each well from column 1 to column 18, and column 23. 45 μL of the inoculum was added to each well from column 19 to column 22 to give the final cell density of 5×10⁵ CFU/mL. The plates were incubated at 37° C. for 18-20h.

Optical density was read at 600 nm (OD600) using Gen5 Spectrophotometer. MIC was determined as the lowest concentration at which OD600 demonstrated 90% growth inhibition compared to growth control. Analysis was performed using Microsoft Excel.

### Synergy Assay

CBD was tested in combination with PMBN in a 14×9 matrix for each. CBD and PMBN were also included on the plate individually (from column 20 and 2) as positive controls and to collect the MIC values.

CBD was two-fold serial diluted across the wells of a 384-wells plate from row B to O (FIG. 1), while PMBN was two-fold serial diluted across the wells of a 384-wells plate from column 2 to 10 (FIG. 2). The inhibitor controls of each antibiotic/compound were 1:2 serial diluted down the plate at column 19, 20, 21 (respectively A, B and C antibiotic).

5 μL of the CBD mother plate were plated vertically intro tested 384-well PS plate (for column 2-10). 5 μL were stamped out horizontally from the PMBN mother plate. Resulting in a final volume of 10 μL in the test plates. 5 μL antibiotic were added to the control column (column 20, 21). Column 23 was the growth control and column 24 was media control.

### Results

Polymyxin B nonapeptide (PMBN) is a compound with poor antimicrobial activity (Table 36).

TABLE 38
MIC test results

| MIC (μg/mL) | |
|---|---|
| Escherichia coli ATCC 25922 | Klebsiella pneumoniae ATCC 700603 |

| | | | | | | | | |
|---|---|---|---|---|---|---|---|---|
| CBD | >256 | >256 | >256 | >256 | >256 | >256 | >256 | >256 |
| PMBN | >32 | >32 | >32 | >32 | >32 | >32 | >32 | >32 |

| | Acinetobacter baumannii ATCC 19606 | | | | Pseudomonas aeruginosa ATCC 27853 | | | |
|---|---|---|---|---|---|---|---|---|
| CBD | >256 | >256 | >256 | >256 | >256 | >256 | >256 | >256 |
| PMBN | >32 | >32 | >32 | >32 | >32 | >32 | >32 | >32 |

Despite its poor antimicrobial inactivity, PMBN is still capable of binding to lipopolysaccharide (LPS), boosting the permeability of the outer membrane of Gram-negative bacteria and increasing bacteria sensibility to hydrophobic antibiotics.

In Tables 37 and 38, CBD in combination with PMBN showed synergistic activity against a panel of wild type (WT) species of Gram-negative bacteria.

TABLE 39
Synergy results

| | CBD MIC (μg/mL) with PMBN | | | | | | | | |
|---|---|---|---|---|---|---|---|---|---|
| | (MCC_9298) at concn (μg/mL) of | | | | | | | FICI | MIC of |
| Strain | CBD | 1 | 2 | 4 | 8 | 16 | 32 | range | PMBN |
| Escherichia | 256 | 64 | 64 | 64 | 64 | 64 | 64 | 0.28-1.25 | >32 |
| coli | 256 | 64 | 64 | >256 | >256 | >256 | 64 | 0.28-1.5 | |
| ATCC 25922 | | | | | | | | | |
| Klebsiella | 256 | >256 | >256 | >256 | >256 | >256 | 64 | 1.03-1.5 | >32 |
| pneumoniae | 256 | >256 | >256 | 64 | 32 | 32 | 32 | 0.38-1.13 | |
| ATCC 700603 | | | | | | | | | |
| Acinetobacter | 256 | 32 | 32 | 32 | 32 | 32 | 32 | 0.16-1.13 | >32 |
| baumannii | 256 | 32 | 32 | 32 | 32 | 32 | 32 | 0.16-1.13 | |
| ATCC 19606 | | | | | | | | | |
| Pseudomonas | 256 | 64 | 64 | 64 | 64 | 64 | 32 | 0.28-1.13 | >32 |
| aeruginosa | 256 | 64 | 64 | 32 | 32 | 32 | 32 | 0.25-1.13 | |
| ATCC 27853 | | | | | | | | | |

TABLE 40
FICI results

| | FICI | | | | | |
|---|---|---|---|---|---|---|
| Strain | 1 | 2 | 4 | 8 | 16 | 32 |
| Escherichia | 0.28125 | 0.3125 | 0.375 | 0.5 | 0.75 | 1.25 |
| coli | 0.28125 | 0.3125 | 1.125 | 1.25 | 1.5 | 1.25 |
| ATCC 25922 | | | | | | |
| Klebsiella | 1.03125 | 1.0625 | 1.125 | 1.25 | 1.5 | 1.25 |
| pneumoniae | 1.03125 | 1.0625 | 0.375 | 0.375 | 0.625 | 1.125 |
| ATCC 700603 | | | | | | |
| Acinetobacter | 0.15625 | 0.1875 | 0.25 | 0.375 | 0.625 | 1.125 |
| baumannii | 0.15625 | 0.1875 | 0.25 | 0.375 | 0.625 | 1.125 |
| ATCC 19606 | | | | | | |
| Pseudomonas | 0.28125 | 0.3125 | 0.375 | 0.5 | 0.75 | 1.125 |
| aeruginosa | 0.28125 | 0.3125 | 0.25 | 0.375 | 0.625 | 1.125 |
| ATCC 27853 | | | | | | |

### Example 11

Minimum Inhibitory Concentrations of CBD Against A. baumannii LPS Deficient Strain

The experiment was performed to investigate the antimicrobial activity of cannabidiol (CBD) against an LPS deficient strain (GN_237 Acinetobacter baumannii, IpxA mutant; Lipid A deficient; obtained by colistin selection from base strain ATCC 19606) and its parent strain (GN_034, Acinetobacter baumannii, ATCC 19606).

The cannabidiol was supplied as dry material. A stock solution was prepared at 1.28 mg/mL in neat DMSO. The highest concentration tested in the assay was 1.28 μg/mL and 2% DMSO was the final concentration using 1/20 dilution to achieve these concentrations.

### Minimum Inhibitory Concentration (MIC) Broth Microdilution

The compound was serially diluted in Cation-adjusted Mueller Hinton Broth (CaMHB; BD, Cat. No. 212322) two-fold across the wells of polystyrene (PS) 96-well plates (Corning; Cat. No. 3370), plated in duplicate. All plates had flat bottom wells and were covered with low-evaporation lids.

Bacteria were cultured in CaMHB at 37° C. overnight, then diluted 40-fold and incubated at 37° C. for a further 2-3 h. The resultant mid-log phase cultures were diluted in CaMHB and added to each well of the compound-containing 96-well plates to give a final cell density of 5×105 CFU/mL, and a final compound concentration range of 0.06-128 μg/mL. The plates were covered and incubated at 37° C. for 20 h.

Two biological replicates ×2 technical replicates were conducted on separate days (final n=4). Inhibition of bacterial growth was determined visually, where the MIC was recorded as the lowest compound concentration with no visible growth.

### Results

Cannabidiol alone was ineffective against parent strain A. baumannii ATCC 19606 (GN_034). However, it was effective against A. baumannii AL1851, an IpxA mutant that is Lipid A deficient (GN_237). It should be noted that, as a result of the lack of Lipid A, A. baumannii AL1851 is resistant to polymyxin B and colistin (which target Lipid A).

TABLE 41
Test results

| | GN_034 | | | | GN_237 | | | |
|---|---|---|---|---|---|---|---|---|
| | Acinetobacter baumannii | | | | Acinetobacter baumannii | | | |
| Compound | ATCC 19606 Type Strain | | | | AL1851, IpxA mutant; Lipid A deficient | | | |
| Colistin Sulfate | 1 | 1 | 1 | 1 | >128 | 128 | >128 | 128 |
| Polymyxin B | >128 | >128 | >128 | >128 | 128 | >128 | >128 | >128 |
| Teicoplanin | >128 | >128 | >128 | >128 | 0.25 | 0.5 | 0.25 | 0.125 |
| Cannabidiol | >128 | >128 | >128 | >128 | 0.25 | 0.25 | 0.125 | 0.125 |
| Gentamycin | 32 | 32 | 64 | 64 | 1 | 1 | 2 | 0.5 |
| Clindamycin | 128 | 128 | >128 | 128 | 4 | 4 | 8 | 8 |
| hydrochloride | | | | | | | | |
| monohydrate | | | | | | | | |
| Mupirocin | 128 | 128 | 128 | 128 | 32 | 16 | 16 | 16 |

The CBD activity against A. baumannii AL1851 confirms that one of the factors leading to a lack of activity by cannabidiol against Gram-negative bacteria is the LPS preventing entry. This result also indicates that CBD may have a potential utility to kill Gram negative bacteria if it is used in combination with LPS suppressors or disruptors.

### Example 12

Cell-Wall Disruption Studies of Cefuroxime in Combination with Cannabidiol

The experiment was conducted to investigate the in vitro activity of the cell-wall disrupting agent cefuroxime in combination with cannabidiol, against wild type species of Gram-negative bacteria.

Methods were as for Example 1. Cannabidiol was tested in combination with cefuroxime.

TABLE 42
Tested cell-wall disrupting agents and compounds

| | | Stock | Max test | Min test |
|---|---|---|---|---|
| | Compound | conc | conc | conc |
| MCC | name | (mg/mL) | (μg/mL) | (μg/mL) |
| MCC_009386 | Cefuroxime | 10 | 256 | 0.0078 |
| MCC_009427 | Cannabidiol | 10 | 256 | 0.0078 |

TABLE 43
Tested bacteria

| ID | Species | Strain | Description |
|---|---|---|---|
| GN_001 | Escherichia | ATCC 25922 | Control, FDA strain |
| | coli | | Seattle 1946 |
| GN_004 | Klebsiella | ATCC 13883 | Type, Polymyxin |
| | pneumoniae | | heteroresistant |
| GN_034 | Acinetobacter | ATCC 19606 | Control, Type strain |
| | baumannii | | |
| GN_042 | Pseudomonas | ATCC 27853 | Control strain |
| | aeruginosa | | |

Cannabidiol combined with cefuroxime is effective against Acinetobacter baumannii and Klebsiella pneumoniae.

TABLE 44
Minimum Inhibitory Concentration (MIC)

| | CBD MIC (μg/mL) with Cefuroxime at concn (μg/mL) of | | | | | | | MIC of |
|---|---|---|---|---|---|---|---|---|
| Strain | CBD | 0.25 | 0.5 | 1 | 2 | 4 | 8 | Cefuroxime |
| Escherichia | 256 | >256 | >256 | >256 | 0.25 | 0.001 | 0.001 | 2 |
| coli | 256 | >256 | >256 | >256 | 0.25 | 0.001 | 0.001 | |
| ATCC 25922 | | | | | | | | |
| | CBD | 1 | 2 | 4 | 8 | 16 | 32 | |
| Klebsiella | 256 | >256 | >256 | >256 | >256 | >256 | 0.001 | 16 |
| pneumoniae | 256 | >256 | >256 | >256 | >256 | >256 | 0.001 | |
| ATCC 700603 | | | | | | | | |
| | CBD | 4 | 8 | 16 | 32 | 64 | 128 | |
| Acinetobacter | 256 | >256 | >256 | 32 | 16 | 0.062 | 0.001 | 64 |
| baumannii | 256 | >256 | >256 | 32 | 32 | 0.062 | 0.001 | |
| ATCC 19606 | | | | | | | | |
| Pseudomonas | 256 | >256 | >256 | >256 | >256 | >256 | >256 | 256 |
| aeruginosa | 256 | >256 | >256 | >256 | >256 | >256 | >256 | |
| ATCC 27853 | | | | | | | | |

TABLE 45
FICI score

| | FICI | | | | | |
|---|---|---|---|---|---|---|
| Strain | 0.25 | 0.5 | 1 | 2 | 4 | 8 |
| Escherichia | 1.125 | 1.25 | 1.5 | 1.0009766 | 2.0000038 | 4.0000038 |
| coli | 1.125 | 1.25 | 1.5 | 1.0009766 | 2.0000038 | 4.0000038 |
| ATCC 25922 | | | | | | |
| | 1 | 2 | 4 | 8 | 16 | 32 |
| Klebsiella | 1.015625 | 1.03125 | 1.0625 | 1.125 | 1.25 | 0.5000038 |
| pneumoniae | 1.015625 | 1.03125 | 1.0625 | 1.125 | 1.25 | 0.5000038 |
| ATCC 700603 | | | | | | |
| | 4 | 8 | 16 | 32 | 64 | 128 |
| Acinetobacter | 1.0625 | 1.125 | 0.375 | 0.5625 | 1.0002422 | 2.0000038 |
| baumannii | 1.0625 | 1.125 | 0.375 | 0.625 | 1.0002422 | 2.0000038 |
| ATCC 19606 | | | | | | |
| Pseudomonas | 1.015625 | 1.03125 | 1.0625 | 1.125 | 1.25 | 1.5 |
| aeruginosa | 1.015625 | 1.03125 | 1.0625 | 1.125 | 1.25 | 1.5 |
| ATCC 27853 | | | | | | |

*FICI scores were calculated using an MIC of 256 μg/mL for cannabidiol.

TABLE 46
Synergy

| | | CBD MIC (μg/mL) with Cefuroxime | | | | | | |
|---|---|---|---|---|---|---|---|---|
| | MIC of | at concentration (μg/mL) of | | | | | | FICI |
| Strain | Cefuroxime | 0.25 | 0.5 | 1 | 2 | 4 | 8 | range |
| Escherichia | 2 | ³32 | ³32 | ³32 | 0.25 | 0.001 | 0.001 | No |
| coli | | | | | | | | Synergy |
| ATCC 25922 | 2 | ³32 | ³32 | ³32 | 0.25 | 0.001 | 0.001 | No |
| | | | | | | | | Synergy |
| | | 1 | 2 | 4 | 8 | 16 | 32 | |
| Klebsiella | 16 | ³32 | ³32 | ³32 | ³32 | ³32 | 0.001 | 0.5-0.125 |
| pneumoniae | 16 | ³32 | ³32 | ³32 | ³32 | ³32 | 0.001 | 0.5-0.125 |
| ATCC 700603 | | | | | | | | |
| | | 4 | 8 | 16 | 32 | 64 | 128 | |
| Acinetobacter | 64 | ³32 | ³32 | 32 | 16 | 0.0625 | 0.001 | 0.38 |
| baumannii | 64 | ³32 | ³32 | 32 | 32 | 0.0625 | 0.001 | 0.38 |
| ATCC 19606 | | | | | | | | |
| Pseudomonas | 256 | ³32 | ³32 | ³32 | ³32 | ³32 | ³32 | No |
| aeruginosa | | | | | | | | Synergy |
| ATCC 27853 | 256 | ³32 | ³32 | ³32 | ³32 | ³32 | ³32 | No |
| | | | | | | | | Synergy |

### Example 13

Cell-Wall Disruption Studies of Ticarcillin in Combination with Cannabidiol

The experiment was conducted to investigate the in vitro activity of ticarcillin in combination with cannabidiol, against wild type species of Gram-negative bacteria.

Methods were as for Example 1. Cannabidiol was tested in combination with ticarcillin (MCC_00094).

TABLE 47
Tested cell-wall disrupting agents and compounds

| Compound | Stock conc | Max test conc | Min test conc |
|---|---|---|---|
| name | (mg/mL) | (μg/mL) | (μg/mL) |
| Ticarcillin | 10 | 16 | 0.00049 |
| Cannabidiol | 10 | 256 | 0.0078 |

TABLE 48
Tested bacteria

| ID | Species | Strain | Description |
|---|---|---|---|
| GN_001 | Escherichia | ATCC 25922 | Control, FDA strain |
| | coli | | Seattle 1946 |
| GN_004 | Klebsiella | ATCC 13883 | Type, Polymyxin |
| | pneumoniae | | heteroresistant |
| GN_034 | Acinetobacter | ATCC 19606 | Control, Type strain |
| | baumannii | | |
| GN_042 | Pseudomonas | ATCC 27853 | Control strain |
| | aeruginosa | | |

Cannabidiol combined with ticarcillin is effective against Escherichia coli, and Pseudomonas aeruginosa.

TABLE 49
Minimum Inhibitory Concentration (MIC)

| | CBD MIC (μg/mL) with Ticarcillin at concn (μg/mL) of | | | | | | | FICI | MIC of |
|---|---|---|---|---|---|---|---|---|---|
| Strain | CBD | 0.25 | 0.5 | 1 | 2 | 4 | 8 | range | Ticarcillin |
| Escherichia | 256 | >256 | >256 | >256 | >256 | 0.125 | 0.001 | 0.5-1.25 | 8 |
| coli | 256 | >256 | >256 | >256 | >256 | 0.125 | 0.001 | 0.5-1.25 | |
| ATCC 25922 | | | | | | | | | |
| | CBD | 2 | 4 | 8 | 16 | 32 | 64 | | |
| Klebsiella | 256 | >256 | >256 | >256 | >256 | >256 | >256 | 1-1.06 | 128 |
| pneumoniae | 256 | >256 | >256 | >256 | >256 | >256 | >256 | 1-1.06 | |
| ATCC 700603 | | | | | | | | | |
| Acinetobacter | 256 | 32 | 16 | 0.001 | 0.001 | 0.001 | 0.001 | 0.63-16 | 4 |
| baumannii | 256 | 32 | 16 | 0.001 | 0.001 | 0.001 | 0.001 | 0.63-16 | |
| ATCC 19606 | | | | | | | | | |
| Pseudomonas | 256 | >256 | >256 | >256 | 0.001 | 0.001 | 0.001 | 0.5-2 | 32/16 |
| aeruginosa | 256 | >256 | >256 | >256 | 0.001 | 0.001 | 0.001 | 1-4 | |
| ATCC 27853 | | | | | | | | | |

TABLE 50
FICI

| | FICI | | | | | |
|---|---|---|---|---|---|---|
| Strain | 0.25 | 0.5 | 1 | 2 | 4 | 8 |
| Escherichia | 1.03125 | 1.0625 | 1.125 | 1.25 | 0.5004883 | 1.0000038 |
| coli | 1.03125 | 1.0625 | 1.125 | 1.25 | 0.5004883 | 1.0000038 |
| ATCC 25922 | | | | | | |
| | 2 | 4 | 8 | 16 | 32 | 64 |
| Klebsiella | 1.0019531 | 1.0039063 | 1.0078125 | 1.015625 | 1.03125 | 1.0625 |
| pneumoniae | 1.0019531 | 1.0039063 | 1.0078125 | 1.015625 | 1.03125 | 1.0625 |
| ATCC 700603 | | | | | | |
| Acinetobacter | 0.625 | 1.0625 | 2.0000038 | 4.0000038 | 8.0000038 | 16.000004 |
| baumannii | 0.625 | 1.0625 | 2.0000038 | 4.0000038 | 8.0000038 | 16.000004 |
| ATCC 19606 | | | | | | |
| Pseudomonas | 1.0625 | 1.125 | 1.25 | 0.5000038 | 1.0000038 | 2.0000038 |
| aeruginosa | 1.125 | 1.25 | 1.5 | 1.0000038 | 2.0000038 | 4.0000038 |
| ATCC 27853 | | | | | | |

TABLE 51
Synergy

| | MIC of | CBD MIC (μg/mL) with Ticarcillin at concentration (μg/mL) of | | | | | | FICI |
|---|---|---|---|---|---|---|---|---|
| Strain | Ticarcillin | 0.25 | 0.5 | 1 | 2 | 4 | 8 | range |
| Escherichia | 8 | ³32 | ³32 | ³32 | ³32 | 0.125 | 0.001 | 0.5 |
| coli | 8 | ³32 | ³32 | ³32 | ³32 | 0.125 | 0.001 | 0.5 |
| ATCC 25922 | | | | | | | | |
| | | 2 | 4 | 8 | 16 | 32 | 64 | |
| Klebsiella | 128 | ³32 | ³32 | ³32 | ³32 | ³32 | ³32 | No |
| pneumoniae | | | | | | | | Synergy |
| ATCC 700603 | 128 | ³32 | ³32 | ³32 | ³32 | ³32 | ³32 | No |
| | | | | | | | | Synergy |
| Acinetobacter | 4 | 32 | 16 | 0.001 | 0.001 | 0.001 | 0.001 | No |
| baumannii | | | | | | | | Synergy |
| ATCC 19606 | 4 | 32 | 16 | 0.001 | 0.001 | 0.001 | 0.001 | No |
| | | | | | | | | Synergy |
| Pseudomonas | 32 | ³32 | ³32 | ³32 | 0.001 | 0.001 | 0.001 | 0.5 |
| aeruginosa | 16 | ³32 | ³32 | ³32 | 0.001 | 0.001 | 0.001 | 0.5 |
| ATCC 27853 | | | | | | | | |

### Example 14

Cell-Wall Disruption Studies of Colistin in Combination with Cannabidiol

The experiment was conducted to investigate the in vitro activity of colistin in combination with cannabidiol, against wild type species of Gram-negative bacteria.

Methods were as for Example 1. Cannabidiol was tested in combination with colistin (MCC_00094).

TABLE 52
Tested cell-wall disrupting agents and compounds

| | | Stock | Max test | Min test |
|---|---|---|---|---|
| | Compound | conc | conc | conc |
| MCC | name | (mg/mL) | (μg/mL) | (μg/mL) |
| MCC_000094 | Colistin | 10 | 16 | 0.00049 |
| MCC_009427 | Cannabidiol | 10 | 256 | 0.0078 |

TABLE 53
Tested bacteria

| ID | Species | Strain | Description |
|---|---|---|---|
| GN_001 | Escherichia | ATCC 25922 | Control, FDA strain |
| | coli | | Seattle 1946 |
| GN_004 | Klebsiella | ATCC 13883 | Type, Polymyxin |
| | pneumoniae | | heteroresistant |
| GN_034 | Acinetobacter | ATCC 19606 | Control, Type strain |
| | baumannii | | |
| GN_042 | Pseudomonas | ATCC 27853 | Control strain |
| | aeruginosa | | |

Cannabidiol combined with colistin is effective against Escherichia coli, Klebsiella pneumoniae and Acinetobacter baumannii.

TABLE 54
Minimum Inhibitory Concentration (MIC)

| | CBD MIC (μg/mL) with Colistin at concn (μg/mL) of | | | | | | | FICI | MIC of |
|---|---|---|---|---|---|---|---|---|---|
| Strain | CBD | 0.015 | 0.03 | 0.06 | 0.125 | 0.25 | 0.5 | range | Colistin |
| Escherichia | 256 | >256 | >256 | 8 | 2 | 1 | 0.125 | 0.15-1.06 | 0.50 |
| coli | 256 | >256 | >256 | 8 | 2 | 1 | 0.125 | 0.15-1.06 | |
| ATCC 25922 | | | | | | | | | |
| | CBD | 0.031 | 0.062 | 0.125 | 0.25 | 0.5 | 1 | | |
| Klebsiella | 256 | >256 | >256 | >256 | >256 | 0.001 | 0.001 | 0.5-1.25 | 0.50 |
| pneumoniae | 256 | >256 | >256 | >256 | >256 | 0.001 | 0.001 | 0.5-1.25 | |
| ATCC 700603 | | | | | | | | | |
| Acinetobacter | 256 | 4 | 2 | 1 | 1 | 0.25 | 0.001 | 0.05-1 | 1.00 |
| baumannii | 256 | 4 | 2 | 1 | 1 | 1 | 0.001 | 0.05-1 | |
| ATCC 19606 | | | | | | | | | |
| Pseudomonas | 256 | >256 | >256 | >256 | >256 | >256 | 0.001 | 1-1.5 | 1.00 |
| aeruginosa | 256 | >256 | >256 | >256 | >256 | >256 | 0.001 | 1-1.5 | |
| ATCC 27853 | | | | | | | | | |

TABLE 55
FICI

| | FICI | | | | | |
|---|---|---|---|---|---|---|
| Strain | 0.015 | 0.03 | 0.06 | 0.125 | 0.25 | 0.5 |
| Escherichia | 1.03 | 1.06 | 0.15125 | 0.2578125 | 0.5039063 | 1.0004883 |
| coli | 1.03 | 1.06 | 0.15125 | 0.2578125 | 0.5039063 | 1.0004883 |
| ATCC 25922 | | | | | | |
| | 0.031 | 0.062 | 0.125 | 0.25 | 0.5 | 1 |
| Klebsiella | 1.03 | 1.06 | 1.12 | 1.25 | 0.5000038 | 1.0000038 |
| pneumoniae | 1.03 | 1.06 | 1.12 | 1.25 | 0.5000038 | 1.0000038 |
| ATCC 700603 | | | | | | |
| Acinetobacter | 0.046625 | 0.0698125 | 0.1289063 | 0.2539063 | 0.5009766 | 1.0000038 |
| baumannii | 0.046625 | 0.0698125 | 0.1289063 | 0.2539063 | 0.5039063 | 1.0000038 |
| ATCC 19606 | | | | | | |
| Pseudomonas | 1.031 | 1.062 | 1.125 | 1.25 | 1.5 | 1.0000038 |
| aeruginosa | 1.031 | 1.062 | 1.125 | 1.25 | 1.5 | 1.0000038 |
| ATCC 27853 | | | | | | |

*FICI scores were calculated using an MIC of 256 μg/mL for cannabidiol.

TABLE 56
Synergy

| | MIC of | CBD MIC (μg/mL) with Colistin at concentration (μg/mL) of | | | | | | FICI |
|---|---|---|---|---|---|---|---|---|
| Strain | Colistin | 0.015 | 0.031 | 0.062 | 0.125 | 0.25 | 0.5 | range |
| Escherichia | 0.5 | ≥32 | ≥32 | 8 | 2 | 1 | 0.125 | 0.15-0.5 |
| coli | 0.5 | ≥32 | ≥32 | 8 | 2 | 1 | 0.125 | 0.15-0.5 |
| ATCC 25922 | | | | | | | | |
| | | 0.031 | 0.062 | 0.125 | 0.25 | 0.5 | 1 | |
| Klebsiella | 0.5 | ≥32 | ≥32 | ≥32 | ≥32 | 0.001 | 0.001 | 0.5-1.25 |
| pneumoniae | 0.5 | ≥32 | ≥32 | ≥32 | ≥32 | 0.001 | 0.001 | 0.5-1.25 |
| ATCC 700603 | | | | | | | | |
| Acinetobacter | 1 | 4 | 2 | 1 | 1 | 0.25 | 0.001 | 0.05-0.5 |
| baumannii | 1 | 4 | 2 | 1 | 1 | 1 | 0.001 | 0.05-0.5 |
| ATCC 19606 | | | | | | | | |
| Pseudomonas | 1 | ≥32 | ≥32 | ≥32 | ≥32 | ≥32 | 0.001 | No |
| aeruginosa | | | | | | | | Synergy |
| ATCC 27853 | 1 | ≥32 | ≥32 | ≥32 | ≥32 | ≥32 | 0.001 | No |
| | | | | | | | | Synergy |

### Example 15

Cell-Wall Disruption Studies of Octapeptin MCC_006442 in Combination with Cannabidiol

The experiment was conducted to investigate the in vitro activity of octapeptin MCC_006442 in combination with cannabidiol, against wild type Gram-negative bacteria.

Methods were as for Example 1. Cannabidiol was tested in combination with octapeptin MCC_006442.

TABLE 57
Tested cell-wall disrupting agents and compounds

| | | Stock | Max test | Min test |
|---|---|---|---|---|
| | Compound | conc | conc | conc |
| MCC | name | (mg/mL) | (μg/mL) | (μg/mL) |
| MCC_006442 | octapeptin | 10 | 128 | 0.00390625 |
| MCC_009427 | Cannabidiol | 10 | 256 | 0.0078 |

TABLE 58
Tested bacteria

| ID | Species | Strain | Description |
|---|---|---|---|
| GN_001 | Escherichia coli | ATCC | Control, FDA strain |
| | | 25922 | Seattle 1946 |
| GN_004 | Klebsiella pneumoniae | ATCC | Type, Polymyxin |
| | | 13883 | heteroresistant |
| GN_034 | Acinetobacter baumannii | ATCC | Control, Type strain |
| | | 19606 | |
| GN_042 | Pseudomonas aeruginosa | ATCC | Control strain |
| | | 27853 | |

Cannabidiol combined with octapeptin MCC_006442 is effective against Escherichia coli, Klebsiella pneumoniae, and Acinetobacter baumannii.

TABLE 59
Minimum Inhibitory Concentration (MIC)

| | CBD MIC (μg/mL) with MCC_6442 at concn (μg/mL) | | | | | | | | |
|---|---|---|---|---|---|---|---|---|---|
| | of | | | | | | | FICI | MIC of |
| Strain | CBD | 0.125 | 0.25 | 0.5 | 1 | 2 | 4 | range | MCC_6442 |
| Escherichia | 256 | >256 | >256 | >256 | >256 | 0.0039 | 0.001 | 0.5-1.25 | 4.00 |
| coli | 256 | >256 | >256 | >256 | >256 | 0.0078 | 0.001 | 0.5-1.25 | |
| ATCC 25922 | | | | | | | | | |
| Klebsiella | 256 | >256 | >256 | >256 | >256 | 1 | 0.25 | 0.25-1.13 | 8/16 |
| pneumoniae | 256 | >256 | >256 | >256 | >256 | 1 | 0.25 | 0.13-1.06 | |
| ATCC 700603 | | | | | | | | | |
| Acinetobacter | 256 | >256 | >256 | >256 | 16 | 1 | 0.5 | 0.07-1.02 | 32.00 |
| baumannii | 256 | >256 | >256 | >256 | 16 | 1 | 0.5 | 0.07-1.02 | |
| ATCC 19606 | | | | | | | | | |
| Pseudomonas | 256 | >256 | >256 | >256 | >256 | >256 | 0.001 | 1-1.5 | 4.00 |
| aeruginosa | 256 | >256 | >256 | >256 | >256 | >256 | 0.001 | 1-1.5 | |
| ATCC 27853 | | | | | | | | | |

TABLE 60
FICI

| | FICI | | | | | |
|---|---|---|---|---|---|---|
| Strain | 0.125 | 0.25 | 0.5 | 1 | 2 | 4 |
| Escherichia coli | 1.03125 | 1.0625 | 1.125 | 1.25 | 0.5000152 | 1.0000038 |
| ATCC 25922 | 1.03125 | 1.0625 | 1.125 | 1.25 | 0.5000305 | 1.0000038 |
| Klebsiella | 1.015625 | 1.03125 | 1.0625 | 1.125 | 0.2539063 | 0.5009766 |
| pneumoniae | 1.0078125 | 1.015625 | 1.03125 | 1.0625 | 0.1289063 | 0.2509766 |
| ATCC 700603 | | | | | | |
| Acinetobacter | 1.0039063 | 1.0078125 | 1.015625 | 0.09375 | 0.0664063 | 0.1269531 |
| baumannii | 1.0039063 | 1.0078125 | 1.015625 | 0.09375 | 0.0664063 | 0.1269531 |
| ATCC 19606 | | | | | | |
| Pseudomonas | 1.03125 | 1.0625 | 1.125 | 1.25 | 1.5 | 1.0000038 |
| aeruginosa | 1.03125 | 1.0625 | 1.125 | 1.25 | 1.5 | 1.0000038 |
| ATCC 27853 | | | | | | |

*FICI scores were calculated using an MIC of 256 μg/mL for cannabidiol.

TABLE 61
Synergy

| | | CBD MIC (μg/mL) with MCC_6442 at concentration | | | | | | |
|---|---|---|---|---|---|---|---|---|
| | | (μg/mL) of | | | | | | |
| | MIC of | | | | | | | FICI |
| Strain | MCC_6442 | 0.125 | 0.25 | 0.5 | 1 | 2 | 4 | range |
| Escherichia coli | 4 | >32 | >32 | >32 | >32 | 0.0039 | 0.001 | 0.5 |
| ATCC 25922 | 4 | >32 | >32 | >32 | >32 | 0.0078 | 0.001 | 0.5 |
| Klebsiella | 8 | >32 | >32 | >32 | >32 | 1 | 0.25 | 0.25-0.5 |
| pneumoniae | 16 | >32 | >32 | >32 | >32 | 1 | 0.25 | 0.13-0.25 |
| ATCC 700603 | | | | | | | | |
| Acinetobacter | 32 | >32 | >32 | >32 | 16 | 1 | 0.5 | 0.09-1.13 |
| baumannii | 32 | >32 | >32 | >32 | 16 | 1 | 0.5 | 0.09-1.13 |
| ATCC 19606 | | | | | | | | |
| Pseudomonas | 4 | >32 | >32 | >32 | >32 | >32 | 0.001 | No |
| aeruginosa | | | | | | | | Synergy |
| ATCC 27853 | 4 | >32 | >32 | >32 | >32 | >32 | 0.001 | No |
| | | | | | | | | Synergy |

### Example 16

Cell-Wall Disruption Studies of Ceftazidime in Combination with Cannabidiol

The experiment was conducted to investigate the in vitro activity of cell-wall disrupting agent ceftazidime in combination with cannabidiol, against wild type Gram-negative bacteria.

Methods were as for Example 1. Cannabidiol was tested in combination with ceftazidime.

TABLE 62
Tested cell-wall disrupting agents and compounds

| | | | Stock | Max test | Min test |
|---|---|---|---|---|---|
| | | Compound | conc | conc | conc |
| MCC | Batch | name | (mg/mL) | (μg/mL) | (μg/mL) |
| MCC_000365 | 001 | Ceftazidime | 10 | 256 | 0.0078125 |
| MCC_009427 | 02 | Cannabidiol | 10 | 256 | 0.0078 |

TABLE 63
Tested bacteria

| ID | Species | Strain | Description |
|---|---|---|---|
| GN_001 | Escherichia coli | ATCC | Control, FDA strain |
| | | 25922 | Seattle 1946 |
| GN_004 | Klebsiella pneumoniae | ATCC | Type, Polymyxin |
| | | 13883 | heteroresistant |
| GN_034 | Acinetobacter baumannii | ATCC | Control, Type strain |
| | | 19606 | |
| GN_042 | Pseudomonas aeruginosa | ATCC | Control strain |
| | | 27853 | |

Cannabidiol combined with ceftazidime is effective against Klebsiella pneumoniae and Pseudomonas aeruginosa.

TABLE 64
Minimum Inhibitory Concentration (MIC)

| | CBD MIC (μg/mL) with Ceftazidime at concn (μg/mL) | | | | | | | | |
|---|---|---|---|---|---|---|---|---|---|
| | of | | | | | | | FICI | MIC of |
| Strain | CBD | 0.015 | 0.031 | 0.062 | 0.125 | 0.25 | 0.5 | range | Ceftazidime |
| Escherichia | 256 | >256 | >256 | >256 | >256 | 0.001 | 0.001 | 1.24-8.06 | 0.06 |
| coli | 256 | >256 | >256 | >256 | >256 | 0.001 | 0.001 | 1.24-8.06 | |
| ATCC 25922 | | | | | | | | | |
| Klebsiella | 256 | >256 | >256 | >256 | >256 | 0.001 | 0.001 | 0-1 | 64/128 |
| pneumoniae | 256 | >256 | >256 | >256 | >256 | 0.001 | 0.001 | 0-1 | |
| ATCC 700603 | | | | | | | | | |
| Acinetobacter | 256 | >256 | >256 | >256 | 32 | 16 | 0.001 | 1.13-4 | 4.00 |
| baumannii | 256 | >256 | >256 | >256 | 32 | 16 | 0.001 | 1.13-4 | |
| ATCC 19606 | | | | | | | | | |
| Pseudomonas | 256 | >256 | >256 | >256 | >256 | 0.001 | 0.001 | 0.5-1.25 | 4.00 |
| aeruginosa | 256 | >256 | >256 | >256 | >256 | 0.001 | 0.001 | 0.5-1.25 | |
| ATCC 27853 | | | | | | | | | |

nt ... Not tested

TABLE 65
FICI

| Strain | FICI | | | | | |
|---|---|---|---|---|---|---|
| | 0.015 | 0.031 | 0.062 | 0.125 | 0.25 | 0.5 |
| Escherichia coli | 1.2419355 | 1.5 | 2 | 3.016129 | 4.0322619 | 8.06452 |
| ATCC 25922 | 1.2419355 | 1.5 | 2 | 3.016129 | 4.0322619 | 8.06452 |
| | 4 | 8 | 16 | 32 | 64 | 128 |
| Klebsiella | 1.0002344 | 1.0004844 | 1.0009688 | 1.0019531 | 0.0039101 | 0.0078163 |
| pneumoniae | 1.0001172 | 1.0002422 | 1.0004844 | 1.0009766 | 0.001957 | 0.0039101 |
| ATCC 700603 | | | | | | |
| | 0.5 | 1 | 2 | 4 | 8 | 16 |
| Acinetobacter | 1.125 | 1.25 | 1.5 | 1.125 | 2.0625 | 4.0000038 |
| baumannii | 1.125 | 1.25 | 1.5 | 1.125 | 2.0625 | 4.0000038 |
| ATCC 19606 | | | | | | |
| | 0.125 | 0.25 | 0.5 | 1 | 2 | 4 |
| Pseudomonas | 1.03125 | 1.0625 | 1.125 | 1.25 | 0.5000038 | 1.0000038 |
| aeruginosa | 1.03125 | 1.0625 | 1.125 | 1.25 | 0.5000038 | 1.0000038 |
| ATCC 27853 | | | | | | |

*FICI scores were calculated using an MIC of 256 μg/mL for cannabidiol.

TABLE 66
Synergy

| | MIC of | CBD MIC (μg/mL) with Ceftazidime at | | | | | | FICI |
|---|---|---|---|---|---|---|---|---|
| Strain | Ceftazidime | concentration (μg/mL) of | | | | | | range |
| | | 0.015 | 0.031 | 0.062 | 0.125 | 0.25 | 0.5 | |
| Escherichia | 0.062 | >32 | >32 | >32 | >32 | 0.001 | 0.001 | No |
| coli | | | | | | | | Synergy |
| ATCC 25922 | 0.062 | >32 | >32 | >32 | >32 | 0.001 | 0.001 | No |
| | | | | | | | | Synergy |
| | | 4 | 8 | 16 | 32 | 64 | 128 | |
| Klebsiella | 64 | >32 | >32 | >32 | >32 | 0.001 | 0.001 | 0.004- |
| pneumoniae | | | | | | | | 0.008 |
| ATCC 700603 | 128 | >32 | >32 | >32 | >32 | 0.001 | 0.001 | 0.002- |
| | | | | | | | | 0.004 |
| | | 0.5 | 1 | 2 | 4 | 8 | 16 | |
| Acinetobacter | 4 | >32 | >32 | >32 | 32 | 16 | 0.001 | No |
| baumannii | | | | | | | | Synergy |
| ATCC 19606 | 4 | >32 | >32 | >32 | 32 | 16 | 0.001 | No |
| | | | | | | | | Synergy |
| | | 0.125 | 0.25 | 0.5 | 1 | 2 | 4 | |
| Pseudomonas | 4 | >32 | >32 | >32 | >32 | 0.001 | 0.001 | 0.5 |
| aeruginosa | | | | | | | | |
| ATCC 27853 | 4 | >32 | >32 | >32 | >32 | 0.001 | 0.001 | 0.5 |

### Example 17

Cell-Wall Disruption Studies of Cefotetan in Combination with Cannabidiol

The experiment was conducted to investigate the in vitro activity of cell-wall disrupting agent cefotetan in combination with cannabidiol, against wild type Gram-negative bacteria.

Methods were as for Example 1. Cannabidiol was tested in combination with cefotetan.

TABLE 67
Tested cell-wall disrupting agents and compounds

| | | | Stock | Max test | Min test |
|---|---|---|---|---|---|
| | | Compound | conc | conc | conc |
| MCC | Batch | name | (mg/mL) | (μg/mL) | (μg/mL) |
| MCC_007251 | 002 | Cefotetan | 10 | 256 | 0.0078125 |
| MCC_009427 | 02 | Cannabidiol | 10 | 256 | 0.0078 |

TABLE 68
Tested bacteria

| ID | Species | Strain | Description |
|---|---|---|---|
| GN_001 | Escherichia coli | ATCC | Control, FDA strain |
| | | 25922 | Seattle 1946 |
| GN_004 | Klebsiella pneumoniae | ATCC | Type, Polymyxin |
| | | 13883 | heteroresistant |
| GN_034 | Acinetobacter baumannii | ATCC | Control, Type strain |
| | | 19606 | |
| GN_042 | Pseudomonas aeruginosa | ATCC | Control strain |
| | | 27853 | |

Cannabidiol combined with cefotetan is effective against Escherichia coli and Klebsiella pneumoniae.

TABLE 69
Minimum Inhibitory Concentration (MIC)

| | | | | | | | | FICI | MIC of |
|---|---|---|---|---|---|---|---|---|---|
| Strain | CBD MIC μg/mL) with Cefotetan at concn (μg/mL) of | | | | | | | range | Cefotetan |
| | CBD | 0.015 | 0.031 | 0.062 | 0.125 | 0.25 | 0.5 | | |
| Escherichia coli | 256 | >256 | >256 | >256 | 0.001 | 0.001 | 0.001 | 0.5-2 | 0.25 |
| ATCC 25922 | 256 | >256 | >256 | >256 | 0.001 | 0.001 | 0.001 | 0.5-2 | |
| | CBD | 0.015 | 0.031 | 0.062 | 0.125 | 0.25 | 0.5 | | |
| Klebsiella | 256 | >256 | >256 | >256 | >256 | >256 | 0.001 | 1-1.5 | 0.5/1.5 |
| pneumoniae | 256 | >256 | >256 | >256 | >256 | >256 | 0.001 | 0.5-1.25 | |
| ATCC 700603 | | | | | | | | | |
| | CBD | 4 | 8 | 16 | 32 | 64 | 128 | | |
| Acinetobacter | 256 | >256 | >256 | >256 | >256 | >256 | >256 | 1.02-1.5 | 256 |
| baumannii | 256 | >256 | >256 | >256 | >256 | >256 | >256 | 1.02-1.5 | |
| ATCC 19606 | | | | | | | | | |
| | CBD | 4 | 8 | 16 | 32 | 64 | 128 | | |
| Pseudomonas | 256 | >256 | >256 | >256 | >256 | >256 | >256 | 1.02-1.5 | 256 |
| aeruginosa | 256 | >256 | >256 | >256 | >256 | >256 | >256 | 1.02-1.5 | |
| ATCC 27853 | | | | | | | | | |

TABLE 70
FICI

| Strain | FICI | | | | | |
|---|---|---|---|---|---|---|
| | 0.015 | 0.031 | 0.062 | 0.125 | 0.25 | 0.5 |
| Escherichia coli | 1.06 | 1.124 | 1.248 | 0.5000038 | 1.0000038 | 2.0000038 |
| ATCC 25922 | 1.06 | 1.124 | 1.248 | 0.5000038 | 1.0000038 | 2.0000038 |
| | 0.015 | 0.031 | 0.062 | 0.125 | 0.25 | 0.5 |
| Klebsiella | | | | | | |
| pneumoniae | 1.03 | 1.062 | 1.124 | 1.25 | 1.5 | 1.0000038 |
| ATCC 700603 | 1.015 | 1.031 | 1.062 | 1.125 | 1.25 | 0.5000038 |
| | 4 | 8 | 16 | 32 | 64 | 128 |
| Acinetobacter | 1.015625 | 1.03125 | 1.0625 | 1.125 | 1.25 | 1.5 |
| baumannii | 1.015625 | 1.03125 | 1.0625 | 1.125 | 1.25 | 1.5 |
| ATCC 19606 | | | | | | |
| | 4 | 8 | 16 | 32 | 64 | 128 |
| Pseudomonas | 1.015625 | 1.03125 | 1.0625 | 1.125 | 1.25 | 1.5 |
| aeruginosa | 1.015625 | 1.03125 | 1.0625 | 1.125 | 1.25 | 1.5 |
| ATCC 27853 | | | | | | |

*FICI scores were calculated using an MIC of 256 μg/mL for cannabidiol.

TABLE 71
Synergy

| | | CBD MIC (μg/mL) with Cefotetan at concentration (μg/mL) | | | | | | |
|---|---|---|---|---|---|---|---|---|
| | | of | | | | | | |
| | MIC of | | | | | | | FICI |
| Strain | Cefotetan | | | | | | | range |
| | | 0.015 | 0.031 | 0.062 | 0.125 | 0.25 | 0.5 | |
| Escherichia | 0.25 | >32 | >32 | >32 | 0.001 | 0.001 | 0.001 | 0.5 |
| coli | | | | | | | | |
| ATCC 25922 | 0.25 | >32 | >32 | >32 | 0.001 | 0.001 | 0.001 | 0.5 |
| Klebsiella | 0.5 | >32 | >32 | >32 | >32 | >32 | 0.001 | No |
| pneumoniae | | | | | | | | Synergy |
| ATCC 700603 | 1 | >32 | >32 | >32 | >32 | >32 | 0.001 | 0.5 |
| | | 4 | 8 | 16 | 32 | 64 | 128 | |
| Acinetobacter | 256 | >32 | >32 | >32 | >32 | >32 | >32 | No |
| baumannii | | | | | | | | Synergy |
| ATCC 19606 | 256 | >32 | >32 | >32 | >32 | >32 | >32 | No |
| | | | | | | | | Synergy |
| Pseudomonas | 256 | >32 | >32 | >32 | >32 | >32 | >32 | No |
| aeruginosa | | | | | | | | Synergy |
| ATCC 27853 | 256 | >32 | >32 | >32 | >32 | >32 | >32 | No |
| | | | | | | | | Synergy |

### Example 18

Cell-Wall Disruption Studies of Aztreonam in Combination with Cannabidiol

The experiment was conducted to investigate the in vitro activity of cell-wall disrupting agent aztreonam in combination with cannabidiol, against wild type Gram-negative bacteria.

Methods were as for Example 1. Cannabidiol was tested in combination with aztreonam.

TABLE 72
Tested cell-wall disrupting agents and compounds

| | | | Stock | Max | |
|---|---|---|---|---|---|
| | | | | test | |
| | | | conc | conc | Min test |
| | | Compound | (mg/ | (μg/ | conc |
| MCC | Batch | name | mL) | mL) | (μg/mL) |
| MCC_007251 | 002 | Aztreonam | 10 | 128 | 0.00390625 |
| MCC_009427 | 02 | Cannabidiol | 10 | 256 | 0.0078 |

TABLE 73
Tested bacteria

| ID | Species | Strain | Description |
|---|---|---|---|
| GN_001 | Escherichia coli | ATCC | Control, FDA strain |
| | | 25922 | Seattle 1946 |
| GN_004 | Klebsiella pneumoniae | ATCC | Type, Polymyxin |
| | | 13883 | heteroresistant |
| GN_034 | Acinetobacter baumannii | ATCC | Control, Type strain |
| | | 19606 | |
| GN_042 | Pseudomonas aeruginosa | ATCC | Control strain |
| | | 27853 | |

Cannabidiol combined with aztreonam is effective against Escherichia coli, Acinetobacter baumannii, Klebsiella pneumoniae and Pseudomonas aeruginosa.

TABLE 74
Minimum Inhibitory Concentration (MIC)

| | CBD MIC (μg/mL) with Aztreonam at concn (μg/mL) | | | | | | | FICI | MIC of |
|---|---|---|---|---|---|---|---|---|---|
| Strain | of | | | | | | | range | Aztreonam |
| | CBD | 0.015 | 0.031 | 0.062 | 0.125 | 0.25 | 0.5 | | |
| Escherichia coli | 256 | >256 | >256 | 0.001 | 0.001 | 0.001 | 0.001 | 0.5-4 | 0.125 |
| ATCC 25922 | 256 | >256 | >256 | 0.001 | 0.001 | 0.001 | 0.001 | 0.5-4 | |
| | CBD | 2 | 4 | 8 | 16 | 32 | 64 | | |
| Klebsiella | 256 | >256 | >256 | >256 | >256 | >256 | 0.001 | 0.01-1 | 64 |
| pneumoniae | 256 | >256 | >256 | >256 | >256 | >256 | 0.001 | 0.01-1 | |
| ATCC 700603 | | | | | | | | | |
| | CBD | 0.5 | 1 | 2 | 4 | 8 | 16 | | |
| Acinetobacter | 256 | >256 | >256 | >256 | 32 | 32 | 0.001 | 0.25-1.06 | 32 |
| baumannii | 256 | >256 | >256 | >256 | 32 | 32 | 0.001 | 0.25-1.06 | |
| ATCC 19606 | | | | | | | | | |
| | CBD | 0.125 | 0.25 | 0.5 | 1 | 2 | 4 | | |
| Pseudomonas | 256 | >256 | >256 | >256 | >256 | 0.1 | 0.001 | 0.5-1.25 | 4 |
| aeruginosa | 256 | >256 | >256 | >256 | >256 | 1 | 0.001 | 0.5-1.25 | |
| ATCC 27853 | | | | | | | | | |

TABLE 75
FICI scores

| Strain | FICI | | | | | |
|---|---|---|---|---|---|---|
| | 0.015 | 0.031 | 0.062 | 0.125 | 0.25 | 0.5 |
| Escherichia coli | 1.12 | 1.248 | 0.4960038 | 1.0000038 | 2.0000038 | 4.0000038 |
| ATCC 25922 | 1.12 | 1.248 | 0.4960038 | 1.0000038 | 2.0000038 | 4.0000038 |
| | 2 | 4 | 8 | 16 | 32 | 64 |
| Klebsiella | 1.0002344 | 1.0004844 | 1.0009688 | 1.0019531 | 1.0039063 | 0.0078163 |
| pneumoniae | 1.0002344 | 1.0004844 | 1.0009688 | 1.0019531 | 1.0039063 | 0.0078163 |
| ATCC 700603 | | | | | | |
| | 0.5 | 1 | 2 | 4 | 8 | 16 |
| Acinetobacter | 1.015625 | 1.03125 | 1.0625 | 0.25 | 0.375 | 0.5000038 |
| baumannii | 1.015625 | 1.03125 | 1.0625 | 0.25 | 0.375 | 0.5000038 |
| ATCC 19606 | | | | | | |
| | 0.125 | 0.25 | 0.5 | 1 | 2 | 4 |
| Pseudomonas | 1.03125 | 1.0625 | 1.125 | 1.25 | 0.5003906 | 1.0000038 |
| aeruginosa | 1.03125 | 1.0625 | 1.125 | 1.25 | 0.5039063 | 1.0000038 |
| ATCC 27853 | | | | | | |

*FICI scores were calculated using an MIC of 256 μg/mL for cannabidiol.

TABLE 76
Synergy

| | MIC of | CBD MIC (μg/mL) with Aztreonam at concentration | | | | | | FICI |
|---|---|---|---|---|---|---|---|---|
| Strain | Aztreonam | (μg/mL) of | | | | | | range |
| | | 0.015 | 0.031 | 0.062 | 0.125 | 0.25 | 0.5 | |
| Escherichia | 0.125 | >32 | >32 | 0.001 | 0.001 | 0.001 | 0.001 | 0.48 |
| coli | | | | | | | | |
| ATCC 25922 | 0.125 | >32 | >32 | 0.001 | 0.001 | 0.001 | 0.001 | 0.48 |
| | | 2 | 4 | 8 | 16 | 32 | 64 | |
| Klebsiella | 64 | >32 | >32 | >32 | >32 | >32 | 0.001 | 0.008 |
| pneumoniae | | | | | | | | |
| ATCC 700603 | 64 | >32 | >32 | >32 | >32 | >32 | 0.001 | 0.008 |
| | | 0.5 | 1 | 2 | 4 | 8 | 16 | |
| Acinetobacter | 32 | >32 | >32 | >32 | 32 | 32 | 0.001 | 0.25- |
| baumannii | | | | | | | | 0.5 |
| ATCC 19606 | 32 | >32 | >32 | >32 | 32 | 32 | 0.001 | 0.25- |
| | | | | | | | | 0.5 |
| | | 0.125 | 0.25 | 0.5 | 1 | 2 | 4 | |
| Pseudomonas | 4 | >32 | >32 | >32 | >32 | 0.5 | 0.001 | 0.5 |
| aeruginosa | | | | | | | | |
| ATCC 27853 | 4 | >32 | >32 | >32 | >32 | 1 | 0.001 | 0.5 |

### Example 19

Cell-Wall Disruption Studies of Cefepime in Combination with Cannabidiol

The experiment was conducted to investigate the in vitro activity of cell-wall disrupting agent cefepime in combination with cannabidiol, against wild type Gram-negative bacteria.

Methods were as for Example 1. Cannabidiol was tested in combination with cefepime.

TABLE 77
Tested cell-wall disrupting agents and compounds

| | | | Stock | Max test | Min test |
|---|---|---|---|---|---|
| | | Compound | conc | conc | conc |
| MCC | Batch | name | (mg/mL) | (μg/mL) | (μg/mL) |
| MCC_007306 | 001 | Cefepime | 10 | 256 | 0.0078125 |
| MCC_009427 | 02 | Cannabidiol | 10 | 256 | 0.0078 |

TABLE 78
Tested bacteria

| ID | Species | Strain | Description |
|---|---|---|---|
| GN_001 | Escherichia coli | ATCC | Control, FDA strain |
| | | 25922 | Seattle 1946 |
| GN_004 | Klebsiella pneumoniae | ATCC | Type, Polymyxin |
| | | 13883 | heteroresistant |
| GN_034 | Acinetobacter baumannii | ATCC | Control, Type strain |
| | | 19606 | |
| GN_042 | Pseudomonas aeruginosa | ATCC | Control strain |
| | | 27853 | |

Cannabidiol combined with cefepime is effective against Escherichia coli and Acinetobacter baumannii.

TABLE 79
Minimum Inhibitory Concentration (MIC)

| | | | | | | | | | MIC of |
|---|---|---|---|---|---|---|---|---|---|
| Strain | CBD MIC (μg/mL) with Cefepime at concn (μg/mL) of | | | | | | | FICI range | Cefepime |
| Escherichia coli | CBD | 0.015 | 0.031 | 0.062 | 0.125 | 0.25 | 0.5 | | |
| ATCC 25922 | 256 | >256 | 0.001 | 0.001 | 0.001 | 0.001 | 0.001 | 1-16.13 | 0.031/0.062 |
| | 256 | >256 | 0.001 | 0.001 | 0.001 | 0.001 | 0.001 | 0.5-8.06 | |
| Klebsiella | CBD | 0.015 | 0.031 | 0.062 | 0.125 | 0.25 | 0.5 | | |
| pneumoniae | 256 | >256 | >256 | >256 | >256 | >256 | >256 | 1.02-1.5 | 1 |
| ATCC 700603 | 256 | >256 | >256 | >256 | >256 | >256 | >256 | 1.02-1.5 | |
| Acinetobacter | CBD | 0.25 | 0.5 | 1 | 2 | 4 | 8 | | |
| baumannii | 256 | >256 | >256 | >256 | >256 | 32 | 16 | 0.38-1.13 | 16 |
| ATCC 19606 | 256 | >256 | >256 | >256 | >256 | 32 | 16 | 0.38-1.13 | |
| Pseudomonas | CBD | 0.015 | 0.031 | 0.062 | 0.125 | 0.25 | 0.5 | | |
| aeruginosa | 256 | >256 | >256 | >256 | >256 | >256 | >256 | 1-1 | 256 |
| ATCC 27853 | 256 | >256 | >256 | >256 | >256 | >256 | >256 | 1-1 | |

nt... Not tested

TABLE 80
FICI scores

| Strain | FICI | | | | | |
|---|---|---|---|---|---|---|
| | 0.015 | 0.031 | 0.062 | 0.125 | 0.25 | 0.5 |
| Escherichia coli | 1.483871 | 1.0000038 | 2.0000038 | 4.0322619 | 8.06452 | 16.129036 |
| ATCC 25922 | 1.2419355 | 0.5000038 | 1.0000038 | 2.0161329 | 4.0322619 | 8.06452 |
| | 0.015 | 0.031 | 0.062 | 0.125 | 0.25 | 0.5 |
| Klebsiella | 1.015 | 1.031 | 1.062 | 1.125 | 1.25 | 1.5 |
| pneumoniae | 1.015 | 1.031 | 1.062 | 1.125 | 1.25 | 1.5 |
| ATCC 700603 | | | | | | |
| | 0.25 | 0.5 | 1 | 2 | 4 | 8 |
| Acinetobacter | 1.015625 | 1.03125 | 1.0625 | 1.125 | 0.375 | 0.5625 |
| baumannii | 1.015625 | 1.03125 | 1.0625 | 1.125 | 0.375 | 0.5625 |
| ATCC 19606 | | | | | | |
| | 0.015 | 0.031 | 0.062 | 0.125 | 0.25 | 0.5 |
| Pseudomonas | 1.0000586 | 1.0001211 | 1.0002422 | 1.0004883 | 1.0009766 | 1.0019531 |
| aeruginosa | 1.0000586 | 1.0001211 | 1.0002422 | 1.0004883 | 1.0009766 | 1.0019531 |
| ATCC 27853 | | | | | | |

*FICI scores were calculated using an MIC of 256 μg/mL for cannabidiol.

TABLE 81
Synergy

| | | CBD MIC (μg/mL) with Cefepime at concentration (μg/mL) of | | | | | | |
|---|---|---|---|---|---|---|---|---|
| | MIC of | | | | | | | FICI |
| Strain | Cefepime | | | | | | | range |
| | | 0.015 | 0.031 | 0.062 | 0.125 | 0.25 | 0.5 | |
| Escherichia | 0.031 | >32 | 0.001 | 0.001 | 0.001 | 0.001 | 0.001 | No |
| coli | | | | | | | | Synergy |
| ATCC 25922 | 0.062 | >32 | 0.001 | 0.001 | 0.001 | 0.001 | 0.001 | 0.5 |
| Klebsiella | 1 | >32 | >32 | >32 | >32 | >32 | >32 | No |
| pneumoniae | | | | | | | | Synergy |
| ATCC 700603 | 1 | >32 | >32 | >32 | >32 | >32 | >32 | No |
| | | | | | | | | Synergy |
| | | 0.25 | 0.5 | 1 | 2 | 4 | 8 | |
| Acinetobacter | 16 | >32 | >32 | >32 | >32 | 32 | 16 | 0.38 |
| baumannii | 16 | >32 | >32 | >32 | >32 | 32 | 16 | 0.38 |
| ATCC 19606 | | | | | | | | |
| | | 0.015 | 0.031 | 0.062 | 0.125 | 0.25 | 0.5 | |
| Pseudomonas | 256 | >32 | >32 | >32 | >32 | >32 | >32 | No |
| aeruginosa | | | | | | | | Synergy |
| ATCC 27853 | 256 | >32 | >32 | >32 | >32 | >32 | >32 | No |
| | | | | | | | | Synergy |

### Example 21

Cell-Wall Disruption Studies of Octapeptin C4 in Combination with Cannabidiol

The experiment was conducted to investigate the in vitro activity of cell-wall disrupting agent octapeptin C4 in combination with cannabidiol, against wild type Gram-negative bacteria.

Methods were as for Example 1. Cannabidiol was tested in combination with octapeptin C4.

TABLE 82
Tested bacteria

| ID | Species | Strain | Description |
|---|---|---|---|
| GN_001 | Escherichia coli | ATCC | Control, FDA strain |
| | | 25922 | Seattle 1946 |
| GN_004 | Klebsiella pneumoniae | ATCC | Type, Polymyxin |
| | | 13883 | heteroresistant |
| GN_034 | Acinetobacter baumannii | ATCC | Control, Type strain |
| | | 19606 | |
| GN_042 | Pseudomonas aeruginosa | ATCC | Control strain |
| | | 27853 | |

Cannabidiol combined with octapeptin C4 is effective against Escherichia coli, Klebsiella pneumoniae, Acinetobacter baumannii, and Pseudomonas aeruginosa.

TABLE 83
Minimum Inhibitory Concentration (MIC)

| | CBD MIC (μg/mL) with Octapeptin C4 | | | | | | | | MIC of |
|---|---|---|---|---|---|---|---|---|---|
| | at concn (μg/mL) of | | | | | | | FICI | Octapeptin |
| Strain | CBD | 0.125 | 0.25 | 0.5 | 1 | 2 | 4 | range | C4 |
| Escherichia coli | 256 | >256 | >256 | >256 | >256 | >256 | 0.062 | 0.5-1.25 | 8 |
| ATCC 25922 | 256 | >256 | >256 | >256 | >256 | 16 | 0.125 | 0.31-1.13 | |
| Klebsiella pneumoniae | 256 | >256 | >256 | >256 | >256 | >256 | 0.5 | 0.5-1.25 | 4/8 |
| ATCC 700603 | 256 | >256 | >256 | >256 | >256 | >256 | 1 | 1-1.5 | |
| Acinetobacter baumannii | 256 | >256 | >256 | >256 | >256 | 8 | 2 | 0.28-1.13 | 8/16 |
| ATCC 19606 | 256 | >256 | >256 | >256 | >256 | 16 | 2 | 0.19-1.06 | |
| Pseudomonas aeruginosa | 256 | >256 | >256 | >256 | >256 | >256 | 0.001 | 0.5-1.25 | 8/4 |
| ATCC 27853 | 256 | >256 | >256 | >256 | >256 | >256 | 0.001 | 1-1.5 | |

TABLE 84
FICI scores

| | FICI | | | | | |
|---|---|---|---|---|---|---|
| Strain | 0.125 | 0.25 | 0.5 | 1 | 2 | 4 |
| Escherichia coli | 1.015625 | 1.03125 | 1.0625 | 1.125 | 1.25 | 0.5002422 |
| ATCC 25922 | 1.015625 | 1.03125 | 1.0625 | 1.125 | 0.3125 | 0.5004883 |
| Klebsiella pneumoniae | 1.015625 | 1.03125 | 1.0625 | 1.125 | 1.25 | 0.5019531 |
| ATCC 700603 | 1.03125 | 1.0625 | 1.125 | 1.25 | 1.5 | 1.0039063 |
| Acinetobacter baumannii | 1.015625 | 1.03125 | 1.0625 | 1.125 | 0.28125 | 0.5078125 |
| ATCC 19606 | 1.0078125 | 1.015625 | 1.03125 | 1.0625 | 0.1875 | 0.2578125 |
| Pseudomonas aeruginosa | 1.015625 | 1.03125 | 1.0625 | 1.125 | 1.25 | 0.5000038 |
| ATCC 27853 | 1.03125 | 1.0625 | 1.125 | 1.25 | 1.5 | 1.0000038 |

*FICI scores were calculated using an MIC of 256 μg/mL for cannabidiol.

TABLE 85
Synergy

| | MIC of | CBD MIC (μg/mL) with Octapeptin C4 | | | | | | |
|---|---|---|---|---|---|---|---|---|
| | Octapeptin | at concentration (μg/mL) of | | | | | | |
| Strain | C4 | 0.125 | 0.25 | 0.5 | 1 | 2 | 4 | FICI range |
| Escherichia coli | 8 | ≥32 | ≥32 | ≥32 | ≥32 | ≥32 | 0.062 | 0.5 |
| ATCC 25922 | 8 | ≥32 | ≥32 | ≥32 | ≥32 | 16 | 0.125 | 0.31-0.5 |
| Klebsiella pneumoniae | 8 | ≥32 | ≥32 | ≥32 | ≥32 | ≥32 | 0.5 | 0.5 |
| ATCC 700603 | 4 | ≥32 | ≥32 | ≥32 | ≥32 | ≥32 | 1 | No Synergy |
| Acinetobacter baumannii | 8 | ≥32 | ≥32 | ≥32 | ≥32 | 8 | 2 | 0.28-0.5 |
| ATCC 19606 | 16 | ≥32 | ≥32 | ≥32 | ≥32 | 16 | 2 | 0.19-0.5 |
| Pseudomonas aeruginosa | 8 | ≥32 | ≥32 | ≥32 | ≥32 | ≥32 | 0.001 | 0.5 |
| ATCC 27853 | 4 | ≥32 | ≥32 | ≥32 | ≥32 | ≥32 | 0.001 | No Synergy |

### Example 22

Cell-Wall Disruption Studies of Spero SPR206 in Combination with Cannabidiol

The experiment was performed to assess the in vitro activity of cell-wall disrupting agent Spero SPR206 in combination with cannabidiol against wild type species of Gram-negative bacteria.

Methods were as for Example 1. Cannabidiol was tested in combination with Spero SPR206. The experiments were carried out three times.

TABLE 86
Tested Bacteria

| ID | Species | Strain | Description |
|---|---|---|---|
| GN_001 | Escherichia coli | ATCC | Control, FDA strain |
| | | 25922 | Seattle 1946 |
| GN_004 | Klebsiella pneumoniae | ATCC | Type, Polymyxin |
| | | 13883 | heteroresistant |
| GN_034 | Acinetobacter baumannii | ATCC | Control, Type strain |
| | | 19606 | |
| GN_042 | Pseudomonas aeruginosa | ATCC | Control strain |
| | | 27853 | |

Cannabidiol in combination with Spero SPR206 was effective against Acinetobacter baumannii, and Escherichia coli.

TABLE 87
Minimal Inhibitory Concentration (MIC)

| | CBD MIC (μg/mL) with Spero SPR206 | | | | | | | | MIC |
|---|---|---|---|---|---|---|---|---|---|
| | at concn (μg/mL) of | | | | | | | FICI | Spero |
| Strain | CBD | 0.125 | 0.25 | 0.5 | 1 | 2 | 4 | range | SPR206 |
| Escherichia coli | 256 | 0.001 | 0.001 | 0.001 | 0.001 | 0.001 | 0.001 | 0.5-16 | 0.250 |
| ATCC 25922 | 256 | 0.001 | 0.001 | 0.001 | 0.001 | 0.001 | 0.001 | 0.5-16 | |
| Klebsiella pneumoniae | 256 | 4 | 0.001 | 0.001 | 0.001 | 0.001 | 0.001 | 0.52-16 | 0.25 |
| ATCC 700603 | 256 | 4 | 0.001 | 0.001 | 0.001 | 0.001 | 0.001 | 0.52-16 | |
| Acinetobacter baumannii | 256 | 0.001 | 0.001 | 0.001 | 0.001 | 0.001 | 0.001 | 0.25-8 | 0.50 |
| ATCC 19606 | 256 | 0.001 | 0.001 | 0.001 | 0.001 | 0.001 | 0.001 | 0.25-8 | |
| Pseudomonas aeruginosa | 256 | >256 | >256 | >256 | 0.001 | 0.001 | 0.001 | 1-4 | 1.00 |
| ATCC 27853 | 256 | >256 | >256 | >256 | 0.001 | 0.001 | 0.001 | 1-4 | |

| | CBD MIC (μg/mL) with Spero SPR206 | | | | | | | | MIC |
|---|---|---|---|---|---|---|---|---|---|
| | at concn (μg/mL) of | | | | | | | FICI | Spero |
| Strain | CBD | 0.007 | 0.015 | 0.031 | 0.062 | 0.125 | 0.25 | range | SPR206 |
| Escherichia coli | 256 | >256 | >256 | >256 | 1 | 1 | 1 | 0.13-1.06 | 0.500 |
| ATCC 25922 | 256 | >256 | >256 | 2 | 1 | 1 | 1 | 0.07-1.03 | |
| Klebsiella pneumoniae | 256 | >256 | >256 | >256 | >256 | 8 | 1 | 0.28-1.12 | 0.50 |
| ATCC 700603 | 256 | >256 | >256 | >256 | >256 | 8 | 1 | 0.28-1.12 | |

| | CBD MIC (μg/mL) with Spero SPR206 | | | | | | | | MIC |
|---|---|---|---|---|---|---|---|---|---|
| | at concn (μg/mL) | | | | | | | FICI | Spero |
| Strain | CBD | 0.125 | 0.25 | 0.5 | 1 | 2 | 4 | range | SPR206 |
| Escherichia coli | 256 | >256 | >256 | 2 | 0.5 | 0.25 | 0.001 | 1.01-8 | 0.500 |
| ATCC 25922 | 256 | >256 | >256 | 2 | 0.25 | 0.25 | 0.0019 | 1.01-8 | |
| Klebsiella pneumoniae | 256 | >256 | >256 | >256 | >256 | 1 | 0.001 | 1.25-8 | 0.50 |
| ATCC 700603 | 256 | >256 | >256 | >256 | >256 | 2 | 0.001 | 1.25-8 | |
| Acinetobacter baumannii | 256 | >256 | >256 | >256 | 1 | 0.001 | 0.001 | 1.5-16 | 0.25 |
| ATCC 19606 | 256 | >256 | >256 | >256 | 0.5 | 0.001 | 0.001 | 1.5-16 | |
| Pseudomonas aeruginosa | 256 | >256 | >256 | >256 | >256 | >256 | >256 | 1.13-5 | 1.00 |
| ATCC 27853 | 256 | >256 | >256 | >256 | >256 | >256 | >256 | 1.13-5 | |

TABLE 88
FICI score

| | FICI | | | | | |
|---|---|---|---|---|---|---|
| Strain | 0.125 | 0.25 | 0.5 | 1 | 2 | 4 |
| Escherichia coli | 0.5000038 | 1.0000038 | 2.0000038 | 4.0000038 | 8.0000038 | 16.000004 |
| ATCC 25922 | 0.5000038 | 1.0000038 | 2.0000038 | 4.0000038 | 8.0000038 | 16.000004 |
| Klebsiella pneumoniae | 0.515625 | 1.0000038 | 2.0000038 | 4.0000038 | 8.0000038 | 16.000004 |
| ATCC 700603 | 0.515625 | 1.0000038 | 2.0000038 | 4.0000038 | 8.0000038 | 16.000004 |
| Acinetobacter baumannii | 0.2500038 | 0.5000038 | 1.0000038 | 2.0000038 | 4.0000038 | 8.0000038 |
| ATCC 19606 | 0.2500038 | 0.5000038 | 1.0000038 | 2.0000038 | 4.0000038 | 8.0000038 |
| Pseudomonas aeruginosa | 1.125 | 1.25 | 1.5 | 1.0000038 | 2.0000038 | 4.0000038 |
| ATCC 27853 | 1.125 | 1.25 | 1.5 | 1.0000038 | 2.0000038 | 4.0000038 |

| | FICI | | | | | |
|---|---|---|---|---|---|---|
| Strain | 0.007 | 0.015 | 0.031 | 0.062 | 0.125 | 0.25 |
| Escherichia coli | 1.014 | 1.03 | 1.062 | 0.1279063 | 0.2539063 | 0.5039063 |
| ATCC 25922 | 1.014 | 1.03 | 0.0698125 | 0.1279063 | 0.2539063 | 0.5039063 |
| Klebsiella pneumoniae | 1.014 | 1.03 | 1.062 | 1.124 | 0.28125 | 0.5039063 |
| ATCC 700603 | 1.014 | 1.03 | 1.062 | 1.124 | 0.28125 | 0.5039063 |

FICI

| Strain | 0.125 | 0.25 | 0.5 | 1 | 2 | 4 |
|---|---|---|---|---|---|---|
| Escherichia coli | 1.25 | 1.5 | 1.0078125 | 2.001953125 | 4.000976563 | 8.000003828 |
| ATCC 25922 | 1.25 | 1.5 | 1.0078125 | 2.000976563 | 4.000976563 | 8.000007422 |
| Klebsiella pneumoniae | 1.25 | 1.5 | 2 | 3 | 4.00390625 | 8.000003828 |
| ATCC 700603 | 1.25 | 1.5 | 2 | 3 | 4.0078125 | 8.000003828 |
| Acinetobacter baumannii | 1.5 | 2 | 3 | 4.00390625 | 8.000003828 | 16.00000383 |
| ATCC 19606 | 1.5 | 2 | 3 | 4.001953125 | 8.000003828 | 16.00000383 |
| Pseudomonas aeruginosa | 1.125 | 1.25 | 1.5 | 2 | 3 | 5 |
| ATCC 27853 | 1.125 | 1.25 | 1.5 | 2 | 3 | 5 |

*FICI scores were calculated using an MIC of 256 μg/mL for cannabidiol.

TABLE 89
Synergy

| | MIC of | CBD MIC (μg/mL) with Spero SPR206 at | | | | | | |
|---|---|---|---|---|---|---|---|---|
| | Spero | concentration (μg/mL) of | | | | | | |
| Strain | SPR206 | 0.125 | 0.25 | 0.5 | 1 | 2 | 4 | FICI range |
| Escherichia coli | 0.25 | 0.001 | 0.001 | 0.001 | 0.001 | 0.001 | 0.001 | 0.5 |
| ATCC 25922 | 0.25 | 0.001 | 0.001 | 0.001 | 0.001 | 0.001 | 0.001 | 0.5 |
| Klebsiella pneumoniae | 0.25 | 4 | 0.001 | 0.001 | 0.001 | 0.001 | 0.001 | No Synergy |
| ATCC 700603 | 0.25 | 4 | 0.001 | 0.001 | 0.001 | 0.001 | 0.001 | No Synergy |
| Acinetobacter baumannii | 0.5 | 0.001 | 0.001 | 0.001 | 0.001 | 0.001 | 0.001 | 0.25-0.5 |
| ATCC 19606 | 0.5 | 0.001 | 0.001 | 0.001 | 0.001 | 0.001 | 0.001 | 0.25-0.5 |
| Pseudomonas aeruginosa | 1 | ≥32 | ≥32 | ≥32 | 0.001 | 0.001 | 0.001 | No Synergy |
| ATCC 27853 | 1 | ≥32 | ≥32 | ≥32 | 0.001 | 0.001 | 0.001 | No Synergy |

| | MIC of | CBD MIC (μg/mL) with Spero SPR206 at | | | | | | |
|---|---|---|---|---|---|---|---|---|
| | Spero | concentration (μg/mL) of | | | | | | |
| Strain | SPR206 | 0.007 | 0.015 | 0.031 | 0.062 | 0.125 | 0.25 | FICI range |
| Escherichia coli | 0.5 | ≥32 | ≥32 | ≥32 | 1 | 1 | 1 | 0.12-0.5 |
| ATCC 25922 | 0.5 | ≥32 | ≥32 | 2 | 1 | 1 | 1 | 0.07-0.5 |
| Klebsiella pneumoniae | 0.5 | ≥32 | ≥32 | ≥32 | ≥32 | 8 | 1 | 0.28-0.5 |
| ATCC 700603 | 0.5 | ≥32 | ≥32 | ≥32 | ≥32 | 8 | 1 | 0.28-0.5 |

| | |
|---|---|
| MIC of | CBD MIC (μg/mL) with Spero SPR206 at |
| Spero | concentration (μg/mL) of |

| Strain | SPR206 | 0.125 | 0.25 | 0.5 | 1 | 2 | 4 | FICI range |
|---|---|---|---|---|---|---|---|---|
| Escherichia coli | 0.5 | >256 | >256 | 2 | 0.5 | 0.25 | 0.001 | 0.07-0.25 |
| ATCC 25922 | 0.5 | >256 | >256 | 2 | 0.25 | 0.25 | 0.0019 | 0.07-0.25 |
| Klebsiella pneumoniae | 0.5 | >256 | >256 | >256 | >256 | 1 | 0.001 | 0.25 |
| ATCC 700603 | 0.5 | >256 | >256 | >256 | >256 | 2 | 0.001 | 0.26 |
| Acinetobacter baumannii | 0.25 | >256 | >256 | >256 | 1 | 0.001 | 0.001 | 0.25 |
| ATCC 19606 | 0.25 | >256 | >256 | >256 | 0.5 | 0.001 | 0.001 | 0.25 |
| Pseudomonas aeruginosa | 1 | >256 | >256 | >256 | >256 | >256 | >256 | No Synergy |
| ATCC 27853 | 1 | >256 | >256 | >256 | >256 | >256 | >256 | No Synergy |

### Example 22

Cell-Wall Disruption Studies of Spero Potentiator SPR741 in Combination with Cannabidiol

The experiment was performed to assess the in vitro activity of cell-wall disrupting agent Spero Potentiator SPR741 in combination with cannabidiol against wild type species of Gram-negative bacteria.

Methods were as for Example 1. Cannabidiol was tested in combination with Spero Potentiator SPR741. The experiments were carried out twice.

TABLE 90
Tested Bacteria

| ID | Species | Strain | Description |
|---|---|---|---|
| GN_001 | Escherichia coli | ATCC | Control, FDA strain |
| | | 25922 | Seattle 1946 |
| GN_004 | Klebsiella pneumoniae | ATCC | Type, Polymyxin |
| | | 13883 | heteroresistant |
| GN_034 | Acinetobacter baumannii | ATCC | Control, Type strain |
| | | 19606 | |
| GN_042 | Pseudomonas aeruginosa | ATCC | Control strain |
| | | 27853 | |

Cannabidiol in combination with Spero Potentiator SPR741 was effective against Escherichia coli, Klebsiella pneumoniae and Acinetobacter baumannii.

TABLE 91
Minimal Inhibitory Concentration (MIC)

| | | | | | | | | | MIC of |
|---|---|---|---|---|---|---|---|---|---|
| | CBD MIC (μg/mL) with Spero Potentiator | | | | | | | | Spero |
| | SPR741 at concn (μg/mL) of | | | | | | | FICI | Potentiator |
| Strain | CBD | 0.5 | 1 | 2 | 4 | 8 | 16 | range | SPR741 |
| Escherichia coli | 256 | >256 | >256 | 1 | 1 | 0.5 | 0.5 | 0.02-1.01 | 128 |
| ATCC 25922 | 256 | >256 | 8 | 0.5 | 0.25 | 0.25 | 0.25 | 0.02-1 | |
| Klebsiella pneumoniae | 256 | >256 | >256 | >256 | >256 | 4 | 0.5 | 0.08-1.03 | >128 |
| ATCC 700603 | 256 | >256 | >256 | >256 | >256 | 8 | 1 | 0.09-1.03 | |
| Acinetobacter baumannii | 256 | >256 | >256 | >256 | >256 | 0.5 | 0.25 | 0.06-1.03 | >128 |
| ATCC 19606 | 256 | >256 | >256 | >256 | >256 | 2 | 0.25 | 0.07-1.03 | |
| Pseudomonas aeruginosa | 256 | >256 | >256 | >256 | >256 | >256 | >256 | 1.01-1.25 | 64.00 |
| ATCC 27853 | 256 | >256 | >256 | >256 | >256 | >256 | >256 | 1.01-1.25 | |

| | | | | | | | | | MIC of |
|---|---|---|---|---|---|---|---|---|---|
| | CBD MIC (μg/mL) with Spero Potentiator | | | | | | | | Spero |
| | SPR741 at concn (μg/mL) of | | | | | | | FICI | Potentiator |
| Strain | CBD | 0.5 | 1 | 2 | 4 | 8 | 16 | range | SPR741 |
| Escherichia coli | 256 | >256 | >256 | 2 | 2 | 1 | 1 | 0.04-1.02 | 64 |
| ATCC 25922 | 256 | >256 | >256 | 2 | 2 | 1 | 1 | 0.04-1.02 | |
| Klebsiella pneumoniae | 256 | >256 | >256 | >256 | >256 | 8 | 4 | 0.16-1.06 | >64 |
| ATCC 700603 | 256 | >256 | >256 | >256 | >256 | 4 | 2 | 0.14-1.06 | |
| Acinetobacter baumannii | 256 | >256 | >256 | >256 | 32 | 2 | 1 | 0.13-1.03 | >64 |
| ATCC 19606 | 256 | >256 | >256 | >256 | 32 | 2 | 1 | 0.13-1.03 | |
| Pseudomonas aeruginosa | 256 | >256 | >256 | >256 | >256 | >256 | >256 | 1.01-1.25 | 64.00 |
| ATCC 27853 | 256 | >256 | >256 | >256 | >256 | >256 | >256 | 1.01-1.25 | |

nt . . . Not tested

TABLE 92
FICI score

| | FICI | | | | | |
|---|---|---|---|---|---|---|
| Strain | 0.5 | 1 | 2 | 4 | 8 | 16 |
| Escherichia coli | 1.0039063 | 1.0078125 | 0.0195313 | 0.0351563 | 0.0644531 | 0.1269531 |
| ATCC 25922 | 1.0039063 | 0.0390625 | 0.0175781 | 0.0322266 | 0.0634766 | 0.1259766 |
| Klebsiella pneumoniae | 1.0039063 | 1.0078125 | 1.015625 | 1.03125 | 0.078125 | 0.1269531 |
| ATCC 700603 | 1.0039063 | 1.0078125 | 1.015625 | 1.03125 | 0.09375 | 0.1289063 |
| Acinetobacter baumannii | 1.0039063 | 1.0078125 | 1.015625 | 1.03125 | 0.0644531 | 0.1259766 |
| ATCC 19606 | 1.0039063 | 1.0078125 | 1.015625 | 1.03125 | 0.0703125 | 0.1259766 |
| Pseudomonas aeruginosa | 1.0078125 | 1.015625 | 1.03125 | 1.0625 | 1.125 | 1.25 |
| ATCC 27853 | 1.0078125 | 1.015625 | 1.03125 | 1.0625 | 1.125 | 1.25 |
| | FICI | | | | | |
| Strain | 0.5 | 1 | 2 | 4 | 8 | 16 |
| Escherichia coli | 1.0078125 | 1.015625 | 0.0390625 | 0.0703125 | 0.1289063 | 0.2539063 |
| ATCC 25922 | 1.0078125 | 1.015625 | 0.0390625 | 0.0703125 | 0.1289063 | 0.2539063 |
| Klebsiella pneumoniae | 1.0078125 | 1.015625 | 1.03125 | 1.0625 | 0.15625 | 0.265625 |
| ATCC 700603 | 1.0078125 | 1.015625 | 1.03125 | 1.0625 | 0.140625 | 0.2578125 |
| Acinetobacter baumannii | 1.0078125 | 1.015625 | 1.03125 | 0.1875 | 0.1328125 | 0.2539063 |
| ATCC 19606 | 1.0078125 | 1.015625 | 1.03125 | 0.1875 | 0.1328125 | 0.2539063 |
| Pseudomonas aeruginosa | 1.0078125 | 1.015625 | 1.03125 | 1.0625 | 1.125 | 1.25 |
| ATCC 27853 | 1.0078125 | 1.015625 | 1.03125 | 1.0625 | 1.125 | 1.25 |

*FICI scores were calculated using an MIC of 256 μg/mL for cannabidiol.

TABLE 93
Synergy

| | MIC of | | | | | | | |
|---|---|---|---|---|---|---|---|---|
| | Spero | CBD MIC (μg/mL) with Spero Potentiator | | | | | | |
| | Potentiator | SPR741 at concentration (μg/mL) of | | | | | | |
| Strain | SPR741 | 0.5 | 1 | 2 | 4 | 8 | 16 | FICI range |
| Escherichia coli | 128 | >256 | >256 | 1 | 1 | 0.5 | 0.5 | 0.02-0.25 |
| ATCC 25922 | 128 | >256 | 8 | 0.5 | 0.25 | 0.25 | 0.25 | 0.02-0.25 |
| Klebsiella pneumoniae | >128 | >256 | >256 | >256 | >256 | 4 | 0.5 | 0.08-0.27 |
| ATCC 700603 | >128 | >256 | >256 | >256 | >256 | 8 | 1 | 0.09-0.27 |
| Acinetobacter baumannii | >128 | >256 | >256 | >256 | >256 | 0.5 | 0.25 | 0.06-0.27 |
| ATCC 19606 | >128 | >256 | >256 | >256 | >256 | 2 | 0.25 | 0.07-0.27 |
| Pseudomonas aeruginosa | 64 | >256 | >256 | >256 | >256 | >256 | >256 | No Synergy |
| ATCC 27853 | 64 | >256 | >256 | >256 | >256 | >256 | >256 | No Synergy |

| | MIC of | | | | | | | |
|---|---|---|---|---|---|---|---|---|
| | Spero | CBD MIC (μg/mL) with Spero Potentiator | | | | | | |
| | Potentiator | SPR741 at concentration (μg/mL) of | | | | | | |
| Strain | SPR741 | 0.5 | 1 | 2 | 4 | 8 | 16 | FICI range |
| Escherichia coli | 64 | ≥32 | ≥32 | 2 | 2 | 1 | 1 | 0.04-0.25 |
| ATCC 25922 | 64 | ≥32 | ≥32 | 2 | 2 | 1 | 1 | 0.04-0.25 |
| Klebsiella pneumoniae | >64 | ≥32 | ≥32 | ≥32 | ≥32 | 8 | 4 | 0.16-0.27 |
| ATCC 700603 | >64 | ≥32 | ≥32 | ≥32 | ≥32 | 4 | 2 | 0.14-0.26 |
| Acinetobacter baumannii | >64 | ≥32 | ≥32 | ≥32 | 32 | 2 | 1 | 0.13-0.25 |
| ATCC 19606 | >64 | ≥32 | ≥32 | ≥32 | 32 | 2 | 1 | 0.13-0.25 |
| Pseudomonas aeruginosa | 64 | ≥32 | ≥32 | ≥32 | ≥32 | ≥32 | ≥32 | No Synergy |
| ATCC 27853 | 64 | ≥32 | ≥32 | ≥32 | ≥32 | ≥32 | ≥32 | No Synergy |

### Example 23

Cell-Wall Disruption Studies of FADDI-287 in Combination with Cannabidiol

The experiment was performed to assess the in vitro activity of cell-wall disrupting agent FADDI-287 in combination with cannabidiol against wild type species of Gram-negative bacteria.

Methods were as for Example 1. Cannabidiol was tested in combination with FADDI-287. DMSO alone was used as a control.

TABLE 94
Tested Bacteria

| ID | Species | Strain | Description |
|---|---|---|---|
| GN_001 | Escherichia coli | ATCC | Control, FDA strain |
| | | 25922 | Seattle 1946 |
| GN_004 | Klebsiella pneumoniae | ATCC | Type, Polymyxin |
| | | 13883 | heteroresistant |
| GN_034 | Acinetobacter baumannii | ATCC | Control, Type strain |
| | | 19606 | |
| GN_042 | Pseudomonas aeruginosa | ATCC | Control strain |
| | | 27853 | |

Cannabidiol in combination with octapeptin FADDI-287 was effective against Klebsiella pneumoniae, Acinetobacter baumannii and Escherichia coli.

TABLE 95
Minimal Inhibitory Concentration (MIC)

| | CBD MIC (μg/mL) with FADDI-287 | | | | | | | | |
|---|---|---|---|---|---|---|---|---|---|
| | at concn (μg/mL) of | | | | | | | FICI | MIC of |
| Strain | CBD | 0.125 | 0.25 | 0.5 | 1 | 2 | 4 | range | FADDI-287 |
| Escherichia coli | 256 | 0.5 | 0.125 | 0.001 | 0.001 | 0.001 | 0.001 | 0.5-16 | 0.250 |
| ATCC 25922 | 256 | 1 | 0.062 | 0.001 | 0.001 | 0.001 | 0.001 | 0.25-8 | |
| Klebsiella pneumoniae | 256 | 8 | 1 | 0.001 | 0.001 | 0.001 | 0.001 | 0.16-4 | 1.00 |
| ATCC 700603 | 256 | 8 | 1 | 0.001 | 0.001 | 0.001 | 0.001 | 0.16-4 | |
| Acinetobacter baumannii | 256 | 1 | 0.015 | 0.001 | 0.001 | 0.001 | 0.001 | 0.25-8 | 0.50 |
| ATCC 19606 | 256 | 1 | 0.015 | 0.001 | 0.001 | 0.001 | 0.001 | 0.25-8 | |
| Pseudomonas aeruginosa | 256 | >256 | >256 | 8 | 0.001 | 0.001 | 0.001 | 0.53-4 | 1.00 |
| ATCC 27853 | 256 | >256 | >256 | 16 | 0.001 | 0.001 | 0.001 | 0.56-4 | |

TABLE 96
FICI score

| | FICI | | | | | |
|---|---|---|---|---|---|---|
| Strain | 0.125 | 0.25 | 0.5 | 1 | 2 | 4 |
| Escherichia coli | 0.5019531 | 1.0004883 | 2.0000038 | 4.0000038 | 8.0000038 | 16.000004 |
| ATCC 25922 | 0.2539063 | 0.5002422 | 1.0000038 | 2.0000038 | 4.0000038 | 8.0000038 |
| Klebsiella pneumoniae | 0.15625 | 0.2539063 | 0.5000038 | 1.0000038 | 2.0000038 | 4.0000038 |
| ATCC 700603 | 0.15625 | 0.2539063 | 0.5000038 | 1.0000038 | 2.0000038 | 4.0000038 |
| Acinetobacter baumannii | 0.2539063 | 0.5000586 | 1.0000038 | 2.0000038 | 4.0000038 | 8.0000038 |
| ATCC 19606 | 0.2539063 | 0.5000586 | 1.0000038 | 2.0000038 | 4.0000038 | 8.0000038 |
| Pseudomonas aeruginosa | 1.125 | 1.25 | 0.53125 | 1.0000038 | 2.0000038 | 4.0000038 |
| ATCC 27853 | 1.125 | 1.25 | 0.5625 | 1.0000038 | 2.0000038 | 4.0000038 |

*FICI scores were calculated using an MIC of 256 μg/mL for cannabidiol.

TABLE 97
Synergy

| | | CBD MIC (μg/mL) with FADDI-287 | | | | | | |
|---|---|---|---|---|---|---|---|---|
| | MIC of | at concentration (μg/mL) of | | | | | | |
| Strain | FADDI-287 | 0.125 | 0.25 | 0.5 | 1 | 2 | 4 | FICI range |
| Escherichia coli | 0.25 | 0.5 | 0.125 | 0.001 | 0.001 | 0.001 | 0.001 | 0.5 |
| ATCC 25922 | 0.5 | 1 | 0.062 | 0.001 | 0.001 | 0.001 | 0.001 | 0.25-0.5 |
| Klebsiella pneumoniae | 1 | 8 | 1 | 0.001 | 0.001 | 0.001 | 0.001 | 0.16-0.5 |
| ATCC 700603 | 1 | 8 | 1 | 0.001 | 0.001 | 0.001 | 0.001 | 0.16-0.5 |
| Acinetobacter baumannii | 0.5 | 1 | 0.015 | 0.001 | 0.001 | 0.001 | 0.001 | 0.25-8 |
| ATCC 19606 | 0.5 | 1 | 0.015 | 0.001 | 0.001 | 0.001 | 0.001 | 0.25-8 |
| Pseudomonas aeruginosa | 1 | ≥32 | ≥32 | 8 | 0.001 | 0.001 | 0.001 | No Synergy |
| ATCC 27853 | 1 | ≥32 | ≥32 | 16 | 0.001 | 0.001 | 0.001 | No Synergy |

### Example 24

Cell-Wall Disruption Studies of MCC_8980 in Combination with Cannabidiol

The experiment was performed to assess the in vitro activity of cell-wall disrupting agent MCC_8980 in combination with cannabidiol against wild type species of Gram-negative bacteria.

Methods were as for Example 1. Cannabidiol was tested in combination with MCC_8980.

TABLE 98
Tested Bacteria

| ID | Species | Strain | Description |
|---|---|---|---|
| GN_001 | Escherichia coli | ATCC | Control, FDA strain |
| | | 25922 | Seattle 1946 |
| GN_004 | Klebsiella pneumoniae | ATCC | Type, Polymyxin |
| | | 13883 | heteroresistant |
| GN_034 | Acinetobacter baumannii | ATCC | Control, Type strain |
| | | 19606 | |
| GN_042 | Pseudomonas aeruginosa | ATCC | Control strain |
| | | 27853 | |

Cannabidiol in combination with MCC_8980 was effective against Klebsiella pneumoniae, Acinetobacter baumannii, Escherichia coli and Pseudomonas aeruginosa.

TABLE 99
Minimal Inhibitory Concentration (MIC)

| | CBD MIC (μg/mL) with MCC_8980 | | | | | | | | |
|---|---|---|---|---|---|---|---|---|---|
| | at concn (μg/mL) of | | | | | | | FICI | MIC of |
| Strain | CBD | 0.5 | 1 | 2 | 4 | 8 | 16 | range | MCC_8980 |
| Escherichia coli | 256 | >256 | >256 | 0.001 | 32 | 2 | 2 | 0.03-1.02 | >64 |
| ATCC 25922 | 256 | >256 | >256 | 0.001 | 16 | 2 | 1 | 0.03-1.02 | |
| Klebsiella pneumoniae | 256 | >256 | >256 | >256 | >256 | >256 | 32 | 0.38-1.13 | 64 |
| ATCC 700603 | 256 | >256 | >256 | >256 | >256 | >256 | 16 | 0.31-1.13 | |
| Acinetobacter baumannii | 256 | >256 | >256 | 8 | 8 | 2 | 1 | 0.06-1.02 | >64 |
| ATCC 19606 | 256 | >256 | >256 | 8 | 8 | 4 | 2 | 0.06-1.02 | |
| Pseudomonas aeruginosa | 256 | >256 | >256 | >256 | >256 | 8 | 1 | 0.16-1.06 | 64 |
| ATCC 27853 | 256 | >256 | >256 | >256 | >256 | 16 | 2 | 0.19-1.06 | |

nt . . . Not tested

TABLE 100
FICI score

| | FICI | | | | | |
|---|---|---|---|---|---|---|
| Strain | 0.5 | 1 | 2 | 4 | 8 | 16 |
| Escherichia coli | 1.0078125 | 1.015625 | 0.0312538 | 0.1875 | 0.1328125 | 0.2578125 |
| ATCC 25922 | 1.0078125 | 1.015625 | 0.0312538 | 0.125 | 0.1328125 | 0.2539063 |
| Klebsiella pneumoniae | 1.0078125 | 1.015625 | 1.03125 | 1.0625 | 1.125 | 0.375 |
| ATCC 700603 | 1.0078125 | 1.015625 | 1.03125 | 1.0625 | 1.125 | 0.3125 |
| Acinetobacter baumannii | 1.0078125 | 1.015625 | 0.0625 | 0.09375 | 0.1328125 | 0.2539063 |
| ATCC 19606 | 1.0078125 | 1.015625 | 0.0625 | 0.09375 | 0.140625 | 0.2578125 |
| Pseudomonas aeruginosa | 1.0078125 | 1.015625 | 1.03125 | 1.0625 | 0.15625 | 0.2539063 |
| ATCC 27853 | 1.0078125 | 1.015625 | 1.03125 | 1.0625 | 0.1875 | 0.2578125 |

*FICI scores were calculated using an MIC of 256 μg/mL for cannabidiol.

TABLE 101
Synergy

| | | CBD MIC (μg/mL) with MCC_8980 | | | | | | |
|---|---|---|---|---|---|---|---|---|
| | MIC of | at concentration (μg/mL) of | | | | | | |
| Strain | MCC_8980 | 0.5 | 1 | 2 | 4 | 8 | 16 | FICI range |
| Escherichia coli | >64 | ≥32 | ≥32 | 0.001 | 32 | 2 | 2 | 0.03-0.26 |
| ATCC 25922 | >64 | ≥32 | ≥32 | 0.001 | 16 | 2 | 1 | 0.03-0.25 |
| Klebsiella pneumoniae | 64 | ≥32 | ≥32 | ≥32 | ≥32 | ≥32 | 32 | 0.38 |
| ATCC 700603 | 64 | ≥32 | ≥32 | ≥32 | ≥32 | ≥32 | 16 | 0.31 |
| Acinetobacter baumannii | >64 | ≥32 | ≥32 | 8 | 8 | 2 | 1 | 0.06-0.25 |
| ATCC 19606 | >64 | ≥32 | ≥32 | 8 | 8 | 4 | 2 | 0.06-0.26 |
| Pseudomonas aeruginosa | 64 | ≥32 | ≥32 | ≥32 | ≥32 | 8 | 1 | 0.16-0.25 |
| ATCC 27853 | 64 | ≥32 | ≥32 | ≥32 | ≥32 | 16 | 2 | 0.19-0.26 |

### REFERENCES

Clinical Laboratory Standards Institute. (2018). Performance Standards for Antimicrobial Disk and Dilution Susceptibility Tests for Bacteria Isolated From Animals; Approved Standard—Fifth Edition VET01. Wayne, P S, USA: CLSI.
Clinical Laboratory Standards Institute. (2018). Performance Standards for Antimicrobial Disk and Dilution Susceptibility Tests for Bacteria Isolated From Animals; Approved Standard—Fourth Edition VET08. Wayne, P S, USA: CLSI.
Clinical Laboratory Standards Institute. (1999). Methods for Determining Bactericidal Activity of Antimicrobial Agents; Approved Guideline M26-A. Wayne, Pa., USA: CLSI.

1. A composition comprising a cannabinoid and a disruptor compound that removes or substantially removes or reduces the integrity of the outer membrane of a bacterium.

2. (canceled)

3. The composition of claim 1 wherein the composition is adapted for administration topically, orally, by injection, or by nasal or pulmonary administration.

4. The composition of claim 1 wherein the cannabinoid is chosen from the list comprising: cannabidiol, cannabinol, cannabigerol, cannabichromene, and Δ9-tetrahydrocannabinol.

5. The composition of claim 1 wherein the disruptor compound that removes or substantially removes or reduces the integrity of the outer membrane of the bacteria is selected from the group consisting of: β-lactams, fosfomycin, lysozyme, polymyxins, lipopeptides including cyclic lipopeptides, chelating agents, glycopeptides, tromethamine, diazaborine, protamine, ketodeoxyoctulosonate analogs, polylysine polymers, polyornithine polymers, nourseothricin, defensins, cecropins, magainins, melittin, bactenecins, seminalplasmin, apidaecin, abaecin, bactericidal/permeability-increasing protein (BPI), eosinophil major basic protein, eosinophil cationic protein (ECP), lactoferrin, azurocidin, cathepsin G, aminoglycosides, Tris, nitrilotriacetate, sodium hexametaphosphate (HMP), acetylsalicylate, ascorbate, fleroxacin, fluoroquinolones, monoglycerides, and immunological agents.

6. A method for the treatment or prevention of an infection by a bacterium in a subject in need of such treatment comprising the step of:
a) administering an effective amount of a composition comprising a cannabinoid and a disruptor compound that removes or substantially removes or reduces the integrity of the outer membrane of a bacterium.

7-8. (canceled)

9. A kit comprising: (i) a cannabinoid and a disruptor compound that removes or substantially removes or reduces the integrity of the outer membrane of a bacteria for the treatment or prevention of a bacterial infection in a subject in need of such treatment or prevention; and (ii) instructions for use.

10. The composition of claim 1, wherein the Gram-negative bacteria is Escherichia coli and the disruptor compound is selected from:
a) Ticarcillin at a cannabidiol:disruptor ratio of more than 1:0.06 to less than 1:8000;
b) Colistin at a cannabidiol:disruptor ratio of more than 1:0.00097 to less than 1:4;
c) MCC_6442 at a cannabidiol:disruptor ratio of more than 1:0.016 to less than 1:4080; d) Cefotetan at a cannabidiol:disruptor ratio of more than 1:0.002 to less than 1:255;
e) Aztreonam at a cannabidiol:disruptor ratio of more than 1:0.00097 to less than 1:127;
f) Octapetin C4 at a cannabidiol:disruptor ratio of more than 1:0.03 to 1:64;
g) Spero SPR206 at a cannabidiol:disruptor ratio of more than 1:0.0004 to less than 1:4080;
h) Spero Potentiator SPR741 at a cannabidiol:disruptor ratio of more than 1:0.002 to 1:64;
i) FADDI-287 at a cannabidiol:disruptor ratio of 1:0.125 to less than 1:510; or
j) MCC_8980 at a cannabidiol:disruptor ratio of more than 1:0.03 to 1:2040.

11. The composition of claim 1, wherein the Gram-negative bacteria is Pseudomonas aeruginosa and the disruptor compound is selected from:
a) Ticarcillin at a cannabidiol:disruptor ratio of more than 1:0.25 to less than 1:32640;
b) Ceftazidime at a cannabidiol:disruptor ratio of more than 1:0.03 to less than 1:4080;
c) Aztreonam at a cannabidiol:disruptor ratio of more than 1:0.03 to less than 1:4080; or
d) MCC_8980 at a cannabidiol:disruptor ratio of more than 1:0.125 to 1:16.

12. The composition of claim 1, wherein the Gram-negative bacteria is Acinetobacter baumannii and the disruptor compound is selected from:
a) Cefuroxime at a cannabidiol:disruptor ratio of more than 1:0.25 to less than 1:2;
b) Colistin at a cannabidiol:disruptor ratio of 1:0.004 to 1:510;
c) MCC_6442 at a cannabidiol:disruptor ratio of more than 1:0.015 to 1:8;
d) Aztreonam at a cannabidiol:disruptor ratio of more than 1:0.06 to 1:16320;
e) Cefepime at a cannabidiol:disruptor ratio of more than 1:0.06 to less than 1:0.5;
f) Octapeptin C4 at a cannabidiol:disruptor ratio of more than 1:0.03 to 1:2;
g) Spero SPR206 at a cannabidiol:disruptor ratio of more than 1:0.002 to less than 1:2040; h) Spero Potentiator SPR741 at a cannabidiol:disruptor ratio of more than 1:0.015 to 1:64;
i) FADDI-287 at a cannabidiol:disruptor ratio of 1:0.125 to less than 1:510; and
j) MCC_8980 at a cannabidiol:disruptor ratio of more than 1:0.125 to 1:16.

13. The composition of claim 1, wherein the Gram-negative bacteria is Klebsiella pneumoniae and the disruptor compound selected from:
a) Colistin at a cannabidiol:disruptor ratio of more than 1:0.008 to less than 1:1020;
b) MCC_6442 at a cannabidiol:disruptor ratio of more than 1:0.03 to 1:16;
c) Spero SPR206 at a cannabidiol:disruptor ratio of more than 1:0.002 to less than 1:4080;
d) Spero Potentiator SPR741 at a cannabidiol:disruptor ratio of more than 1:0.016 to 1:32;
e) FADDI-287 at a cannabidiol:disruptor ratio of 1:0.125 to less than 1:510;
f) MCC_8980 at a cannabidiol:disruptor ratio of more than 1:0.25 to 1:2.
